(19) United States
(12) Patent Application Publication
(10) Pub. No.: US 2025/0223553 A1
(43) Pub. Date: Jul. 10, 2025

(54) METHODS AND COMPOSITIONS FOR STEM CELL DIFFERENTIATION

(71) Applicant: GC Therapeutics, Inc., Cambridge, MA (US)

(72) Inventors: Maxence O. DELLACHERIE, Cambridge, MA (US); Anna ROBERTS, Cambridge, MA (US); Ian M. LI, Cambridge, MA (US); Lucy S. CHONG, Cambridge, MA (US); Cory J. SMITH, Cambridge, MA (US); Parastoo KHOSHAKHLAGH, Cambridge, MA (US); Hon Man Alex NG, Cambridge, MA (US)

(21) Appl. No.: 19/003,147

(22) Filed: Dec. 27, 2024

Related U.S. Application Data
(63) Continuation of application No. PCT/US2023/069550, filed on Jun. 30, 2023.

(30) Foreign Application Priority Data
Jun. 30, 2022 (GB) 2209676.2

(51) Int. Cl. C12N 5/0783; C12N 5/0789

(52) U.S. Cl. CPC C12N 5/0636; C12N 5/0647; C12N 2506/45; C12N 2510/00

(57) ABSTRACT

The present disclosure provides methods and compositions for stem cell differentiation. In some examples, the stem cell is a pluripotent stem cell (PSC). The PSC may comprise a nucleic acid comprising an open reading frame encoding one or more transcription factors, one or more transcription factors, or an activator of transcription of the open reading frame encoding one or more transcription factors.

### CROSS REFERENCE

This application is a continuation of International Application No. PCT/US2023/069550, filed on Jun. 30, 2023, which claims benefit of priority to United Kingdom Patent Application No. 2209676.2 filed Jun. 30, 2022, the entirety of which is hereby incorporated by reference herein.

### SEQUENCE LISTING

The present application is being filed along with a Sequence Listing in electronic format. The Sequence Listing is provided as a file entitled 57963-707_601_SL.XML, created on Jun. 30, 2023, which is 2,513 bytes in size. The information in the electronic format of the Sequence Listing is incorporated by reference in its entirety.

### BACKGROUND

Induced pluripotent stem cells (iPSCs) can be programmed into various phenotypes via the introduction of one or more transcription factors or one or more molecules that modulate transcription or transcription factors. For example, iPSCs can be programmed into immune cells, which may be used in, for example, immunotherapeutic applications.

### SUMMARY

In an aspect, the present disclosure provides a pluripotent stem cell (PSC) comprising a nucleic acid comprising an open reading frame encoding one or more transcription factors, one or more transcription factors, or an activator of transcription of the open reading frame encoding one or more transcription factors, wherein the nucleic acid comprising an open reading frame encoding one or more transcription factors, the one or more transcription factors, or the activator of transcription of the open reading frame encoding one or more transcription factors induces differentiation of the PSC into an immune cell in 28 days or less. In some embodiments, wherein the immune cell expresses FOXP3. In some embodiments, wherein the immune cell expresses CD34. In some embodiments, wherein the immune cell expresses CD45. In some embodiments, wherein the immune cell expresses CD4. In some embodiments, wherein the immune cell is a regulatory T cell. In some embodiments, wherein the immune cell is a hematopoietic progenitor. In some embodiments, wherein the immune cell is a leukocyte. In some embodiments, wherein the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, wherein two or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, wherein three or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, wherein four or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, wherein the nucleic acid comprises two or more open reading frame encoding one or more transcription factors. In some embodiments, wherein the one or more transcription factors are selected from the group of transcription factor families consisting of Zinc fingers C2H2-type, BAF complex, Runt-related transcription factors, BTB domain containing, basic helix-loop-helix (bHLH), Nuclear factor-KB, and basic helix-loop-helix. In some embodiments, wherein the one or more transcription factors are selected from the group of transcription factor families consisting of Zinc fingers C2H2-type, Ring finger proteins, Forkhead boxes, Notch receptors, MicroRNA protein coding host genes, GATA zinc finger domain containing, Nuclear factors of activated T-cells, IPT domain containing, TCF/LEF transcription factor family, Wnt enhanceosome complex, NF-kappa B complex subunits, IPT domain containing, Runt-related transcription factors, CUT class homeoboxes and pseudogenes, and BTB domain containing. In some embodiments, wherein the one or more transcription factors are selected from the group of transcription factor families consisting of Zinc fingers C2H2-type, BAF complex, Ring finger proteins, Forkhead boxes, GATA zinc finger domain containing, Nuclear factors of activated T-cells, IPT domain containing, phosphatase 1 regulatory subunits, TCF/LEF transcription factor family, Wnt enhanceosome complex, Notch receptors, MicroRNA protein coding host genes, RAR related orphan receptors, CUT class homeoboxes and pseudogenes, T-box transcription factors, Basic helix-loop-helix proteins, and BTB domain containing. In some embodiments, wherein the one or more transcription factors are selected from the group of transcription factor families consisting of Forkhead boxes, GATA zinc finger domain containing, Zinc fingers C2H2-type, phosphatase 1 regulatory subunits, Interferon regulatory factors, Kruppel like factors, Notch receptors, MicroRNA protein coding host genes, NF-kappa B complex subunits, IPT domain containing, Basic helix-loop-helix proteins, and BTB domain containing. In some embodiments, wherein the one or more transcription factors are selected from the group of transcription factor families consisting of Zinc fingers C2H2-type, BAF complex, Ring finger proteins, Forkhead boxes, GATA zinc finger domain containing, Kruppel like factors, Nuclear factors of activated T-cells, IPT domain containing, MicroRNA protein coding host genes, Notch receptors, NF-kappa B complex subunits, IPT domain containing, CUT class homeoboxes and pseudogenes, T-box transcription factors, Basic helix-loop-helix proteins, TCF/LEF transcription factor family, and Wnt enhanceosome complex. In some embodiments, wherein the one or more transcription factors are selected from the group of transcription factor families consisting of Forkhead boxes, GATA zinc finger domain containing, Zinc fingers C2H2-type, Kruppel like factors, TCF/LEF transcription factor family, Wnt enhanceosome complex, Notch receptors, MicroRNA protein coding host genes, RAR related orphan receptors, NF-kappa B complex subunits, IPT domain containing, Runt-related transcription factors, Basic helix-loop-helix proteins. In some embodiments, wherein the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, Notch receptors, MicroRNA protein coding host genes, Runt-related transcription factors, Basic helix-loop-helix proteins, TCF/LEF transcription factor family, Wnt enhanceosome complex, BTB domain containing. In some embodiments, wherein the one or more transcription factors are selected from the group of transcription factor families consisting of Ring finger proteins, Forkhead boxes, Zinc fingers C2H2-type Protein, phosphatase 1 regulatory subunits, Interferon regulatory factors, Nuclear factors of activated T-cells, IPT domain containing, MicroRNA protein coding host genes, Notch receptors, MicroRNA protein coding host genes, RAR related orphan receptors, NF-kappa B complex subunits, IPT domain containing, CUT class homeoboxes and pseudogenes, Basic helix-loop-helix proteins. In some embodiments, wherein the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, IPT domain containing, T-box transcription factors, TCF/LEF transcription factor family, Wnt enhanceosome complex, BTB domain containing, Zinc fingers C2H2-type. In some embodiments, wherein the one or more transcription factors are selected from the group of transcription factor families consisting of Ring finger proteins, Forkhead boxes, GATA zinc finger domain containing, Interferon regulatory factors, Zinc fingers C2H2-type, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, CUT class homeoboxes and pseudogenes, Basic helix-loop-helix proteins, TCF/LEF transcription factor family, Wnt enhanceosome complex, BTB domain containing. In some embodiments, wherein the one or more transcription factors are selected from the group of transcription factor families consisting of Zinc fingers C2H2-type, BAF complex, Forkhead boxes, Zinc fingers C2H2-type Protein, phosphatase 1 regulatory subunits, Kruppel like factors, TCF/LEF transcription factor family, Wnt enhanceosome complex, Nuclear factors of activated T-cells, IPT domain containing, Notch receptors, MicroRNA protein coding host genes, and T-box transcription factors. In some embodiments, wherein the one or more transcription factors are selected from the group of transcription factor families consisting of Forkhead boxes, GATA zinc finger domain containing, Zinc fingers C2H2-type, TCF/LEF transcription factor family, Wnt enhanceosome complex, Nuclear factors of activated T-cells, IPT domain containing, MicroRNA protein coding host genes, Notch receptors, RAR related orphan receptors, and Basic helix-loop-helix proteins. In some embodiments, wherein the one or more transcription factors are selected from the group of transcription factor families consisting of Forkhead boxes, GATA zinc finger domain containing, Zinc fingers C2H2-type, phosphatase 1 regulatory subunits, Kruppel like factors, TCF/LEF transcription factor family, Wnt enhanceosome complex, Nuclear factors of activated T-cells, IPT domain containing, MicroRNA protein coding host genes, Runt-related transcription factors, CUT class homeoboxes and pseudogenes, and T-box transcription factors, Basic helix-loop-helix proteins. In some embodiments, wherein the one or more transcription factors are selected from the group of transcription factor families consisting of Zinc fingers C2H2-type, BAF complex, Forkhead boxes, GATA zinc finger domain containing, phosphatase 1 regulatory subunits, Interferon regulatory factors, Kruppel like factors, Nuclear factors of activated T-cells, IPT domain containing, MicroRNA protein coding host genes, NF-kappa B complex subunits, Runt-related transcription factors, CUT class homeoboxes and pseudogenes, and BTB domain containing. In some embodiments, wherein the one or more transcription factors are selected from the group of transcription factor families consisting of Ring finger proteins, Forkhead boxes, GATA zinc finger domain containing, Zinc fingers C2H2-type Protein, phosphatase 1 regulatory subunits, TCF/LEF transcription factor family, Wnt enhanceosome complex, Nuclear factors of activated T-cells, IPT domain containing, MicroRNA protein coding host genes, RAR related orphan receptors, Runt-related transcription factors, T-box transcription factors, Basic helix-loop-helix proteins, and BTB domain containing. In some embodiments, wherein the one or more transcription factors are selected from the group of transcription factor families consisting of Forkhead boxes, GATA zinc finger domain containing, Zinc fingers C2H2-type Protein, phosphatase 1 regulatory subunits, Kruppel like factors, Notch receptors, MicroRNA protein coding host genes, NF-kappa B complex subunits, IPT domain containing, and Basic helix-loop-helix proteins. In some embodiments, wherein the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, MicroRNA protein coding host genes, Notch receptors, Runt-related transcription factors, Basic helix-loop-helix proteins, TCF/LEF transcription factor family, Wnt enhanceosome complex, and BTB domain containing. In some embodiments, wherein the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, T-box transcription factors, TCF/LEF transcription factor family, Wnt enhanceosome complex, and BTB domain containing. In some embodiments, the one or more transcription factors comprise any one of the transcription factor combinations in Tables 1, 2, 3, 5, 6, 7, 8, 9, 10, or 11. In some embodiments, wherein the PSC is provided in a media. In some embodiments, wherein the nucleic acid comprising an open reading frame encoding one or more transcription factors, the one or more transcription factors, or the activator of transcription of the open reading frame encoding one or more transcription factors induces differentiation of the PSC into an immune cell in 11 days or less, 5 days or less, 4 days or less, 1 day or less. In some embodiments, wherein the media is not altered during the differentiation of the PSC into the immune cell.

In another aspect, the present disclosure provides a population of cells comprising two or more of the immune cell as disclosed herein. In some embodiments, the cells are adherent cells. In some embodiments, the cells are suspension cells. In some embodiments, at least 5% of the cells express FOXP3. In some embodiments, at least 10% of the cells express FOXP3. In some embodiments, at least 20% of the cells express FOXP3. In some embodiments, at least 30% of the cells express FOXP3. In some embodiments, at least 40% of the cells express FOXP3. In some embodiments, at least 1% of the cells express CD45. In some embodiments, at least 3% of the cells express CD45. In some embodiments, at least 3% of the cells express CD45. In some embodiments, at least 4% of the cells express CD45. In some embodiments, at least 5% of the cells express CD45. In some embodiments, at least 6% of the cells express CD45. In some embodiments, at least 1% of the cells express CD34. In some embodiments, at least 2% of the cells express CD34. In some embodiments, at least 2.5% of the cells express CD34. In some embodiments, at least 2% of the cells express CD4. In some embodiments, at least 3% of the cells express CD4. In some embodiments, at least 4% of the cells express CD4. In some embodiments, at least 4% of the cells express CD4. In some embodiments, no nutrient, growth factor or microenvironmental/matrix optimizations are performed.

In another aspect, the present disclosure provides a pluripotent stem cell (PSC) comprising a nucleic acid comprising an open reading frame encoding one or more transcription factors, one or more transcription factors, or an activator of transcription of the open reading frame encoding one or more transcription factors, wherein the nucleic acid comprising an open reading frame encoding one or more transcription factors, the one or more transcription factors, or the activator of transcription of the open reading frame encoding one or more transcription factors induce differentiation of the PSC into an immune cell, wherein the PSC is provided in a media, and wherein the media is not altered during the differentiation of the PSC into the immune cell. In some embodiments, wherein the immune cell expresses FOXP3. In some embodiments, wherein the immune cell expresses CD34. In some embodiments, wherein the immune cell expresses CD45. In some embodiments, wherein the immune cell expresses CD4. In some embodiments, wherein the immune cell is a regulatory T cell. In some embodiments, wherein the immune cell is a hematopoietic progenitor. In some embodiments, wherein the immune cell is a leukocyte. In some embodiments, wherein the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, wherein two or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, wherein three or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, wherein four or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, wherein the nucleic acid comprises two or more open reading frame encoding one or more transcription factors. In some embodiments, wherein the one or more transcription factors are selected from the group of transcription factor families consisting of Zinc fingers C2H2-type, BAF complex, Runt-related transcription factors, BTB domain containing, basic helix-loop-helix (bHLH), Nuclear factor-KB, and basic helix-loop-helix. In some embodiments, wherein the one or more transcription factors are selected from the group of transcription factor families consisting of Zinc fingers C2H2-type, Ring finger proteins, Forkhead boxes, Notch receptors, MicroRNA protein coding host genes, GATA zinc finger domain containing, Nuclear factors of activated T-cells, IPT domain containing, TCF/LEF transcription factor family, Wnt enhanceosome complex, NF-kappa B complex subunits, IPT domain containing, Runt-related transcription factors, CUT class homeoboxes and pseudogenes, and BTB domain containing. In some embodiments, wherein the one or more transcription factors are selected from the group of transcription factor families consisting of Zinc fingers C2H2-type, BAF complex, Ring finger proteins, Forkhead boxes, GATA zinc finger domain containing, Nuclear factors of activated T-cells, IPT domain containing, phosphatase 1 regulatory subunits, TCF/LEF transcription factor family, Wnt enhanceosome complex, Notch receptors, MicroRNA protein coding host genes, RAR related orphan receptors, CUT class homeoboxes and pseudogenes, T-box transcription factors, Basic helix-loop-helix proteins, and BTB domain containing. In some embodiments, wherein the one or more transcription factors are selected from the group of transcription factor families consisting of Forkhead boxes, GATA zinc finger domain containing, Zinc fingers C2H2-type, phosphatase 1 regulatory subunits, Interferon regulatory factors, Kruppel like factors, Notch receptors, MicroRNA protein coding host genes, NF-kappa B complex subunits, IPT domain containing, Basic helix-loop-helix proteins, and BTB domain containing. In some embodiments, wherein the one or more transcription factors are selected from the group of transcription factor families consisting of Zinc fingers C2H2-type, BAF complex, Ring finger proteins, Forkhead boxes, GATA zinc finger domain containing, Kruppel like factors, Nuclear factors of activated T-cells, IPT domain containing, MicroRNA protein coding host genes, Notch receptors, NF-kappa B complex subunits, IPT domain containing, CUT class homeoboxes and pseudogenes, T-box transcription factors, Basic helix-loop-helix proteins, TCF/LEF transcription factor family, and Wnt enhanceosome complex. In some embodiments, wherein the one or more transcription factors are selected from the group of transcription factor families consisting of Forkhead boxes, GATA zinc finger domain containing, Zinc fingers C2H2-type, Kruppel like factors, TCF/LEF transcription factor family, Wnt enhanceosome complex, Notch receptors, MicroRNA protein coding host genes, RAR related orphan receptors, NF-kappa B complex subunits, IPT domain containing, Runt-related transcription factors, Basic helix-loop-helix proteins. In some embodiments, wherein the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, Notch receptors, MicroRNA protein coding host genes, Runt-related transcription factors, Basic helix-loop-helix proteins, TCF/LEF transcription factor family, Wnt enhanceosome complex, BTB domain containing. In some embodiments, wherein the one or more transcription factors are selected from the group of transcription factor families consisting of Ring finger proteins, Forkhead boxes, Zinc fingers C2H2-type Protein, phosphatase 1 regulatory subunits, Interferon regulatory factors, Nuclear factors of activated T-cells, IPT domain containing, MicroRNA protein coding host genes, Notch receptors, MicroRNA protein coding host genes, RAR related orphan receptors, NF-kappa B complex subunits, IPT domain containing, CUT class homeoboxes and pseudogenes, Basic helix-loop-helix proteins. In some embodiments, wherein the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, IPT domain containing, T-box transcription factors, TCF/LEF transcription factor family, Wnt enhanceosome complex, BTB domain containing, Zinc fingers C2H2-type. In some embodiments, wherein the one or more transcription factors are selected from the group of transcription factor families consisting of Ring finger proteins, Forkhead boxes, GATA zinc finger domain containing, Interferon regulatory factors, Zinc fingers C2H2-type, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, CUT class homeoboxes and pseudogenes, Basic helix-loop-helix proteins, TCF/LEF transcription factor family, Wnt enhanceosome complex, BTB domain containing. In some embodiments, wherein the one or more transcription factors are selected from the group of transcription factor families consisting of Zinc fingers C2H2-type, BAF complex, Forkhead boxes, Zinc fingers C2H2-type Protein, phosphatase 1 regulatory subunits, Kruppel like factors, TCF/LEF transcription factor family, Wnt enhanceosome complex, Nuclear factors of activated T-cells, IPT domain containing, Notch receptors, MicroRNA protein coding host genes, and T-box transcription factors. In some embodiments, wherein the one or more transcription factors are selected from the group of transcription factor families consisting of Forkhead boxes, GATA zinc finger domain containing, Zinc fingers C2H2-type, TCF/LEF transcription factor family, Wnt enhanceosome complex, Nuclear factors of activated T-cells, IPT domain containing, MicroRNA protein coding host genes, Notch receptors, RAR related orphan receptors, and Basic helix-loop-helix proteins. In some embodiments, wherein the one or more transcription factors are selected from the group of transcription factor families consisting of Forkhead boxes, GATA zinc finger domain containing, Zinc fingers C2H2-type, phosphatase 1 regulatory subunits, Kruppel like factors, TCF/LEF transcription factor family, Wnt enhanceosome complex, Nuclear factors of activated T-cells, IPT domain containing, MicroRNA protein coding host genes, Runt-related transcription factors, CUT class homeoboxes and pseudogenes, and T-box transcription factors, Basic helix-loop-helix proteins. In some embodiments, wherein the one or more transcription factors are selected from the group of transcription factor families consisting of Zinc fingers C2H2-type, BAF complex, Forkhead boxes, GATA zinc finger domain containing, phosphatase 1 regulatory subunits, Interferon regulatory factors, Kruppel like factors, Nuclear factors of activated T-cells, IPT domain containing, MicroRNA protein coding host genes, NF-kappa B complex subunits, Runt-related transcription factors, CUT class homeoboxes and pseudogenes, and BTB domain containing. In some embodiments, wherein the one or more transcription factors are selected from the group of transcription factor families consisting of Ring finger proteins, Forkhead boxes, GATA zinc finger domain containing, Zinc fingers C2H2-type Protein, phosphatase 1 regulatory subunits, TCF/LEF transcription factor family, Wnt enhanceosome complex, Nuclear factors of activated T-cells, IPT domain containing, MicroRNA protein coding host genes, RAR related orphan receptors, Runt-related transcription factors, T-box transcription factors, Basic helix-loop-helix proteins, and BTB domain containing. In some embodiments, wherein the one or more transcription factors are selected from the group of transcription factor families consisting of Forkhead boxes, GATA zinc finger domain containing, Zinc fingers C2H2-type Protein, phosphatase 1 regulatory subunits, Kruppel like factors, Notch receptors, MicroRNA protein coding host genes, NF-kappa B complex subunits, IPT domain containing, and Basic helix-loop-helix proteins. In some embodiments, wherein the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, MicroRNA protein coding host genes, Notch receptors, Runt-related transcription factors, Basic helix-loop-helix proteins, TCF/LEF transcription factor family, Wnt enhanceosome complex, and BTB domain containing. In some embodiments, wherein the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, T-box transcription factors, TCF/LEF transcription factor family, Wnt enhanceosome complex, and BTB domain containing. In some embodiments, the one or more transcription factors comprise any one of the transcription factor combinations in Tables 1, 2, 3, 5, 6, 7, 8, 9, 10, or 11. In some embodiments, wherein the nucleic acid comprising an open reading frame encoding one or more transcription factors, the one or more transcription factors, or the activator of transcription of the open reading frame encoding one or more transcription factors induces differentiation of the PSC into an immune cell in 28 days or less. In some embodiments, wherein the nucleic acid comprising an open reading frame encoding one or more transcription factors, the one or more transcription factors, or the activator of transcription of the open reading frame encoding one or more transcription factors induces differentiation of the PSC into an immune cell in 11 days or less, 5 days or less, 4 days or less, 1 day or less.

In another aspect, the present disclosure provides a population of cells comprising two or more of the immune cell as disclosed herein. In some embodiments, the cells are adherent cells. In some embodiments, the cells are suspension cells. In some embodiments, at least 5% of the cells express FOXP3. In some embodiments, at least 10% of the cells express FOXP3. In some embodiments, at least 20% of the cells express FOXP3. In some embodiments, at least 30% of the cells express FOXP3. In some embodiments, at least 40% of the cells express FOXP3. In some embodiments, at least 1% of the cells express CD45. In some embodiments, at least 3% of the cells express CD45. In some embodiments, at least 3% of the cells express CD45. In some embodiments, at least 4% of the cells express CD45. In some embodiments, at least 5% of the cells express CD45. In some embodiments, at least 6% of the cells express CD45. In some embodiments, at least 1% of the cells express CD34. In some embodiments, at least 2% of the cells express CD34. In some embodiments, at least 2.5% of the cells express CD34. In some embodiments, at least 2% of the cells express CD4. In some embodiments, at least 3% of the cells express CD4. In some embodiments, at least 4% of the cells express CD4. In some embodiments, at least 4% of the cells express CD4. In some embodiments, no nutrient, growth factor or microenvironmental/matrix optimizations are performed.

In another aspect, the present disclosure provides a method of generating a population of immune cells, the method comprising: providing one or more pluripotent stem cells (PSCs); expressing in the one or more PSCs a nucleic acid comprising an open reading frame encoding one or more transcription factors, one or more transcription factors, or an activator of transcription of the open reading frame encoding one or more transcription factors; and generating the population of immune cells from the one or more PSCs. In some embodiments, at least one of the immune cells expresses FOXP3. In some embodiments, at least one of the immune cells expresses CD34. In some embodiments, at least one of the immune cells expresses CD45. In some embodiments, at least one of the immune cells expresses CD4. In some embodiments, at least one of the immune cells is a regulatory T cell. In some embodiments, at least one of the immune cells is a hematopoietic progenitor. In some embodiments, at least one of the immune cells is a leukocyte. In some embodiments, the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, two or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, three or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, four or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the nucleic acid comprises two or more open reading frame encoding one or more transcription factors. In some embodiments, the one or more transcription factors are selected from the group of transcription factor families consisting of Zinc fingers C2H2-type, BAF complex, Runt-related transcription factors, BTB domain containing, basic helix-loop-helix (bHLH), Nuclear factor-KB, and basic helix-loop-helix. In some embodiments, the one or more transcription factors are selected from the group of transcription factor families consisting of Zinc fingers C2H2-type, Ring finger proteins, Forkhead boxes, Notch receptors, MicroRNA protein coding host genes, GATA zinc finger domain containing, Nuclear factors of activated T-cells, IPT domain containing, TCF/LEF transcription factor family, Wnt enhanceosome complex, NF-kappa B complex subunits, IPT domain containing, Runt-related transcription factors, CUT class homeoboxes and pseudogenes, and BTB domain containing. In some embodiments, the one or more transcription factors are selected from the group of transcription factor families consisting of Zinc fingers C2H2-type, BAF complex, Ring finger proteins, Forkhead boxes, GATA zinc finger domain containing, Nuclear factors of activated T-cells, IPT domain containing, phosphatase 1 regulatory subunits, TCF/LEF transcription factor family, Wnt enhanceosome complex, Notch receptors, MicroRNA protein coding host genes, RAR related orphan receptors, CUT class homeoboxes and pseudogenes, T-box transcription factors, Basic helix-loop-helix proteins, and BTB domain containing. In some embodiments, the one or more transcription factors are selected from the group of transcription factor families consisting of Forkhead boxes, GATA zinc finger domain containing, Zinc fingers C2H2-type, phosphatase 1 regulatory subunits, Interferon regulatory factors, Kruppel like factors, Notch receptors, MicroRNA protein coding host genes, NF-kappa B complex subunits, IPT domain containing, Basic helix-loop-helix proteins, and BTB domain containing. In some embodiments, the one or more transcription factors are selected from the group of transcription factor families consisting of Zinc fingers C2H2-type, BAF complex, Ring finger proteins, Forkhead boxes, GATA zinc finger domain containing, Kruppel like factors, Nuclear factors of activated T-cells, IPT domain containing, MicroRNA protein coding host genes, Notch receptors, NF-kappa B complex subunits, IPT domain containing, CUT class homeoboxes and pseudogenes, T-box transcription factors, Basic helix-loop-helix proteins, TCF/LEF transcription factor family, and Wnt enhanceosome complex. In some embodiments, the one or more transcription factors are selected from the group of transcription factor families consisting of Forkhead boxes, GATA zinc finger domain containing, Zinc fingers C2H2-type, Kruppel like factors, TCF/LEF transcription factor family, Wnt enhanceosome complex, Notch receptors, MicroRNA protein coding host genes, RAR related orphan receptors, NF-kappa B complex subunits, IPT domain containing, Runt-related transcription factors, Basic helix-loop-helix proteins. In some embodiments, the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, Notch receptors, MicroRNA protein coding host genes, Runt-related transcription factors, Basic helix-loop-helix proteins, TCF/LEF transcription factor family, Wnt enhanceosome complex, BTB domain containing. In some embodiments, the one or more transcription factors are selected from the group of transcription factor families consisting of Ring finger proteins, Forkhead boxes, Zinc fingers C2H2-type Protein, phosphatase 1 regulatory subunits, Interferon regulatory factors, Nuclear factors of activated T-cells, IPT domain containing, MicroRNA protein coding host genes, Notch receptors, MicroRNA protein coding host genes, RAR related orphan receptors, NF-kappa B complex subunits, IPT domain containing, CUT class homeoboxes and pseudogenes, Basic helix-loop-helix proteins. In some embodiments, the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, IPT domain containing, T-box transcription factors, TCF/LEF transcription factor family, Wnt enhanceosome complex, BTB domain containing, Zinc fingers C2H2-type. In some embodiments, the one or more transcription factors are selected from the group of transcription factor families consisting of Ring finger proteins, Forkhead boxes, GATA zinc finger domain containing, Interferon regulatory factors, Zinc fingers C2H2-type, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, CUT class homeoboxes and pseudogenes, Basic helix-loop-helix proteins, TCF/LEF transcription factor family, Wnt enhanceosome complex, BTB domain containing. In some embodiments, the one or more transcription factors are selected from the group of transcription factor families consisting of Zinc fingers C2H2-type, BAF complex, Forkhead boxes, Zinc fingers C2H2-type Protein, phosphatase 1 regulatory subunits, Kruppel like factors, TCF/LEF transcription factor family, Wnt enhanceosome complex, Nuclear factors of activated T-cells, IPT domain containing, Notch receptors, MicroRNA protein coding host genes, and T-box transcription factors. In some embodiments, the one or more transcription factors are selected from the group of transcription factor families consisting of Forkhead boxes, GATA zinc finger domain containing, Zinc fingers C2H2-type, TCF/LEF transcription factor family, Wnt enhanceosome complex, Nuclear factors of activated T-cells, IPT domain containing, MicroRNA protein coding host genes, Notch receptors, RAR related orphan receptors, and Basic helix-loop-helix proteins. In some embodiments, the one or more transcription factors are selected from the group of transcription factor families consisting of Forkhead boxes, GATA zinc finger domain containing, Zinc fingers C2H2-type, phosphatase 1 regulatory subunits, Kruppel like factors, TCF/LEF transcription factor family, Wnt enhanceosome complex, Nuclear factors of activated T-cells, IPT domain containing, MicroRNA protein coding host genes, Runt-related transcription factors, CUT class homeoboxes and pseudogenes, and T-box transcription factors, Basic helix-loop-helix proteins. In some embodiments, the one or more transcription factors are selected from the group of transcription factor families consisting of Zinc fingers C2H2-type, BAF complex, Forkhead boxes, GATA zinc finger domain containing, phosphatase 1 regulatory subunits, Interferon regulatory factors, Kruppel like factors, Nuclear factors of activated T-cells, IPT domain containing, MicroRNA protein coding host genes, NF-kappa B complex subunits, Runt-related transcription factors, CUT class homeoboxes and pseudogenes, and BTB domain containing. In some embodiments, the one or more transcription factors are selected from the group of transcription factor families consisting of Ring finger proteins, Forkhead boxes, GATA zinc finger domain containing, Zinc fingers C2H2-type Protein, phosphatase 1 regulatory subunits, TCF/LEF transcription factor family, Wnt enhanceosome complex, Nuclear factors of activated T-cells, IPT domain containing, MicroRNA protein coding host genes, RAR related orphan receptors, Runt-related transcription factors, T-box transcription factors, Basic helix-loop-helix proteins, and BTB domain containing. In some embodiments, the one or more transcription factors are selected from the group of transcription factor families consisting of Forkhead boxes, GATA zinc finger domain containing, Zinc fingers C2H2-type Protein, phosphatase 1 regulatory subunits, Kruppel like factors, Notch receptors, MicroRNA protein coding host genes, NF-kappa B complex subunits, IPT domain containing, and Basic helix-loop-helix proteins. In some embodiments, the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, MicroRNA protein coding host genes, Notch receptors, Runt-related transcription factors, Basic helix-loop-helix proteins, TCF/LEF transcription factor family, Wnt enhanceosome complex, and BTB domain containing. In some embodiments, the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, T-box transcription factors, TCF/LEF transcription factor family, Wnt enhanceosome complex, and BTB domain containing. In some embodiments, the one or more PSCs are provided in a media. In some embodiments, the media is not altered during the generation of the population of immune cells from the one or more PSCs. In some embodiments, the one or more transcription factors comprise any one of the transcription factor combinations in Tables 1, 2, 3, 5, 6, 7, 8, 9, 10, or 11. In some embodiments, the nucleic acid comprising an open reading frame encoding one or more transcription factors, the one or more transcription factors, or the activator of transcription of the open reading frame encoding one or more transcription factors induces differentiation of the one or more PSCs into the population of immune cells in 28 days or less. In some embodiments, the nucleic acid comprising an open reading frame encoding one or more transcription factors, the one or more transcription factors, or the activator of transcription of the open reading frame encoding one or more transcription factors induces differentiation of the one or more PSCs into the population of immune cells in 11 days or less, 5 days or less, 4 days or less, 1 day or less.

Another aspect of the present disclosure provides a population of cells comprising two or more of the immune cells as disclosed herein. In some embodiments, the cells are adherent cells. In some embodiments, the cells are suspension cells. In some embodiments, at least 5% of the cells express FOXP3. In some embodiments, at least 10% of the cells express FOXP3. In some embodiments, at least 20% of the cells express FOXP3. In some embodiments, at least 30% of the cells express FOXP3. In some embodiments, at least 40% of the cells express FOXP3. In some embodiments, at least 1% of the cells express CD45. In some embodiments, at least 3% of the cells express CD45. In some embodiments, at least 3% of the cells express CD45. In some embodiments, at least 4% of the cells express CD45. In some embodiments, at least 5% of the cells express CD45. In some embodiments, at least 6% of the cells express CD45. In some embodiments, at least 1% of the cells express CD34. In some embodiments, at least 2% of the cells express CD34. In some embodiments, at least 2.5% of the cells express CD34. In some embodiments, at least 2% of the cells express CD4. In some embodiments, at least 3% of the cells express CD4. In some embodiments, at least 4% of the cells express CD4. In some embodiments, at least 4% of the cells express CD4. In some embodiments, no nutrient, growth factor or microenvironmental/matrix optimizations are performed.

In another aspect, the present disclosure provides a method of generating a population of immune cells comprising: providing one or more pluripotent stem cells (PSCs); inducing in the one or more PSCs the expression of a nucleic acid comprising an open reading frame encoding one or more transcription factors, one or more transcription factors, or an activator of transcription of the open reading frame encoding one or more transcription factors, wherein the nucleic acid, wherein the induction occurs in 28 days or less; and generating the population of immune cells from the one or more PSCs. In some embodiments, at least one of the immune cells expresses FOXP3. In some embodiments, at least one of the immune cells expresses CD34. In some embodiments, at least one of the immune cells expresses CD45. In some embodiments, at least one of the immune cells expresses CD4. In some embodiments, at least one of the immune cells is a regulatory T cell. In some embodiments, at least one of the immune cells is a hematopoietic progenitor. In some embodiments, at least one of the immune cells is a leukocyte. In some embodiments, the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, two or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, three or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, four or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the nucleic acid comprises two or more open reading frame encoding one or more transcription factors. In some embodiments, the one or more transcription factors are selected from the group of transcription factor families consisting of Zinc fingers C2H2-type, BAF complex, Runt-related transcription factors, BTB domain containing, basic helix-loop-helix (bHLH), Nuclear factor-KB, and basic helix-loop-helix. In some embodiments, the one or more transcription factors are selected from the group of transcription factor families consisting of Zinc fingers C2H2-type, Ring finger proteins, Forkhead boxes, Notch receptors, MicroRNA protein coding host genes, GATA zinc finger domain containing, Nuclear factors of activated T-cells, IPT domain containing, TCF/LEF transcription factor family, Wnt enhanceosome complex, NF-kappa B complex subunits, IPT domain containing, Runt-related transcription factors, CUT class homeoboxes and pseudogenes, and BTB domain containing. In some embodiments, the one or more transcription factors are selected from the group of transcription factor families consisting of Zinc fingers C2H2-type, BAF complex, Ring finger proteins, Forkhead boxes, GATA zinc finger domain containing, Nuclear factors of activated T-cells, IPT domain containing, phosphatase 1 regulatory subunits, TCF/LEF transcription factor family, Wnt enhanceosome complex, Notch receptors, MicroRNA protein coding host genes, RAR related orphan receptors, CUT class homeoboxes and pseudogenes, T-box transcription factors, Basic helix-loop-helix proteins, and BTB domain containing. In some embodiments, the one or more transcription factors are selected from the group of transcription factor families consisting of Forkhead boxes, GATA zinc finger domain containing, Zinc fingers C2H2-type, phosphatase 1 regulatory subunits, Interferon regulatory factors, Kruppel like factors, Notch receptors, MicroRNA protein coding host genes, NF-kappa B complex subunits, IPT domain containing, Basic helix-loop-helix proteins, and BTB domain containing. In some embodiments, the one or more transcription factors are selected from the group of transcription factor families consisting of Zinc fingers C2H2-type, BAF complex, Ring finger proteins, Forkhead boxes, GATA zinc finger domain containing, Kruppel like factors, Nuclear factors of activated T-cells, IPT domain containing, MicroRNA protein coding host genes, Notch receptors, NF-kappa B complex subunits, IPT domain containing, CUT class homeoboxes and pseudogenes, T-box transcription factors, Basic helix-loop-helix proteins, TCF/LEF transcription factor family, and Wnt enhanceosome complex. In some embodiments, the one or more transcription factors are selected from the group of transcription factor families consisting of Forkhead boxes, GATA zinc finger domain containing, Zinc fingers C2H2-type, Kruppel like factors, TCF/LEF transcription factor family, Wnt enhanceosome complex, Notch receptors, MicroRNA protein coding host genes, RAR related orphan receptors, NF-kappa B complex subunits, IPT domain containing, Runt-related transcription factors, Basic helix-loop-helix proteins. In some embodiments, the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, Notch receptors, MicroRNA protein coding host genes, Runt-related transcription factors, Basic helix-loop-helix proteins, TCF/LEF transcription factor family, Wnt enhanceosome complex, BTB domain containing. In some embodiments, the one or more transcription factors are selected from the group of transcription factor families consisting of Ring finger proteins, Forkhead boxes, Zinc fingers C2H2-type Protein, phosphatase 1 regulatory subunits, Interferon regulatory factors, Nuclear factors of activated T-cells, IPT domain containing, MicroRNA protein coding host genes, Notch receptors, MicroRNA protein coding host genes, RAR related orphan receptors, NF-kappa B complex subunits, IPT domain containing, CUT class homeoboxes and pseudogenes, Basic helix-loop-helix proteins. In some embodiments, the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, IPT domain containing, T-box transcription factors, TCF/LEF transcription factor family, Wnt enhanceosome complex, BTB domain containing, Zinc fingers C2H2-type. In some embodiments, the one or more transcription factors are selected from the group of transcription factor families consisting of Ring finger proteins, Forkhead boxes, GATA zinc finger domain containing, Interferon regulatory factors, Zinc fingers C2H2-type, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, CUT class homeoboxes and pseudogenes, Basic helix-loop-helix proteins, TCF/LEF transcription factor family, Wnt enhanceosome complex, BTB domain containing. In some embodiments, the one or more transcription factors are selected from the group of transcription factor families consisting of Zinc fingers C2H2-type, BAF complex, Forkhead boxes, Zinc fingers C2H2-type Protein, phosphatase 1 regulatory subunits, Kruppel like factors, TCF/LEF transcription factor family, Wnt enhanceosome complex, Nuclear factors of activated T-cells, IPT domain containing, Notch receptors, MicroRNA protein coding host genes, and T-box transcription factors. In some embodiments, the one or more transcription factors are selected from the group of transcription factor families consisting of Forkhead boxes, GATA zinc finger domain containing, Zinc fingers C2H2-type, TCF/LEF transcription factor family, Wnt enhanceosome complex, Nuclear factors of activated T-cells, IPT domain containing, MicroRNA protein coding host genes, Notch receptors, RAR related orphan receptors, and Basic helix-loop-helix proteins. In some embodiments, the one or more transcription factors are selected from the group of transcription factor families consisting of Forkhead boxes, GATA zinc finger domain containing, Zinc fingers C2H2-type, phosphatase 1 regulatory subunits, Kruppel like factors, TCF/LEF transcription factor family, Wnt enhanceosome complex, Nuclear factors of activated T-cells, IPT domain containing, MicroRNA protein coding host genes, Runt-related transcription factors, CUT class homeoboxes and pseudogenes, and T-box transcription factors, Basic helix-loop-helix proteins. In some embodiments, the one or more transcription factors are selected from the group of transcription factor families consisting of Zinc fingers C2H2-type, BAF complex, Forkhead boxes, GATA zinc finger domain containing, phosphatase 1 regulatory subunits, Interferon regulatory factors, Kruppel like factors, Nuclear factors of activated T-cells, IPT domain containing, MicroRNA protein coding host genes, NF-kappa B complex subunits, Runt-related transcription factors, CUT class homeoboxes and pseudogenes, and BTB domain containing. In some embodiments, the one or more transcription factors are selected from the group of transcription factor families consisting of Ring finger proteins, Forkhead boxes, GATA zinc finger domain containing, Zinc fingers C2H2-type Protein, phosphatase 1 regulatory subunits, TCF/LEF transcription factor family, Wnt enhanceosome complex, Nuclear factors of activated T-cells, IPT domain containing, MicroRNA protein coding host genes, RAR related orphan receptors, Runt-related transcription factors, T-box transcription factors, Basic helix-loop-helix proteins, and BTB domain containing. In some embodiments, the one or more transcription factors are selected from the group of transcription factor families consisting of Forkhead boxes, GATA zinc finger domain containing, Zinc fingers C2H2-type Protein, phosphatase 1 regulatory subunits, Kruppel like factors, Notch receptors, MicroRNA protein coding host genes, NF-kappa B complex subunits, IPT domain containing, and Basic helix-loop-helix proteins. In some embodiments, the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, MicroRNA protein coding host genes, Notch receptors, Runt-related transcription factors, Basic helix-loop-helix proteins, TCF/LEF transcription factor family, Wnt enhanceosome complex, and BTB domain containing. In some embodiments, the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, T-box transcription factors, TCF/LEF transcription factor family, Wnt enhanceosome complex, and BTB domain containing. In some embodiments, the one or more transcription factors comprise any one of the transcription factor combinations in Tables 1, 2, 3, 5, 6, 7, 8, 9, 10, or 11. In some embodiments, the one or more PSCs are provided in a media. In some embodiments, the media is not altered during the generation of the population of immune cells from the one or more PSCs. In some embodiments, the nucleic acid comprising an open reading frame encoding one or more transcription factors, the one or more transcription factors, or the activator of transcription of the open reading frame encoding one or more transcription factors induces differentiation of the one or more PSCs into the population of immune cells in 11 days or less, 5 days or less, 4 days or less, 1 day or less.

Another aspect of the present disclosure provides a population of cells comprising two or more of the immune cells as disclosed herein. In some embodiments, the cells are adherent cells. In some embodiments, the cells are suspension cells. In some embodiments, at least 5% of the cells express FOXP3. In some embodiments, at least 10% of the cells express FOXP3. In some embodiments, at least 20% of the cells express FOXP3. In some embodiments, at least 30% of the cells express FOXP3. In some embodiments, at least 40% of the cells express FOXP3. In some embodiments, at least 1% of the cells express CD45. In some embodiments, at least 3% of the cells express CD45. In some embodiments, at least 3% of the cells express CD45. In some embodiments, at least 4% of the cells express CD45. In some embodiments, at least 5% of the cells express CD45. In some embodiments, at least 6% of the cells express CD45. In some embodiments, at least 1% of the cells express CD34. In some embodiments, at least 2% of the cells express CD34. In some embodiments, at least 2.5% of the cells express CD34. In some embodiments, at least 2% of the cells express CD4. In some embodiments, at least 3% of the cells express CD4. In some embodiments, at least 4% of the cells express CD4. In some embodiments, at least 4% of the cells express CD4. In some embodiments, no nutrient, growth factor or microenvironmental/matrix optimizations are performed.

In another aspect, the present disclosure provides an immune cell comprising a nucleic acid comprising an open reading frame encoding one or more transcription factors, one or more transcription factors, or an activator of transcription of the open reading frame encoding one or more transcription factors, wherein the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the immune cell expresses FOXP3. In some embodiments, the immune cell expresses CD34. In some embodiments, the immune cell expresses CD45. In some embodiments, the immune cell expresses CD4. In some embodiments, the immune cell is a regulatory T cell. In some embodiments, the immune cell is a hematopoietic progenitor. In some embodiments, the immune cell is a leukocyte. In some embodiments, the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, two or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, three or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, four or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the nucleic acid comprises two or more open reading frame encoding one or more transcription factors. In some embodiments, the one or more transcription factors are selected from the group of transcription factor families consisting of Zinc fingers C2H2-type, BAF complex, Runt-related transcription factors, BTB domain containing, basic helix-loop-helix (bHLH), Nuclear factor-KB, and basic helix-loop-helix. In some embodiments, the one or more transcription factors are selected from the group of transcription factor families consisting of Zinc fingers C2H2-type, Ring finger proteins, Forkhead boxes, Notch receptors, MicroRNA protein coding host genes, GATA zinc finger domain containing, Nuclear factors of activated T-cells, IPT domain containing, TCF/LEF transcription factor family, Wnt enhanceosome complex, NF-kappa B complex subunits, IPT domain containing, Runt-related transcription factors, CUT class homeoboxes and pseudogenes, and BTB domain containing. In some embodiments, the one or more transcription factors are selected from the group of transcription factor families consisting of Zinc fingers C2H2-type, BAF complex, Ring finger proteins, Forkhead boxes, GATA zinc finger domain containing, Nuclear factors of activated T-cells, IPT domain containing, phosphatase 1 regulatory subunits, TCF/LEF transcription factor family, Wnt enhanceosome complex, Notch receptors, MicroRNA protein coding host genes, RAR related orphan receptors, CUT class homeoboxes and pseudogenes, T-box transcription factors, Basic helix-loop-helix proteins, and BTB domain containing. In some embodiments, the one or more transcription factors are selected from the group of transcription factor families consisting of Forkhead boxes, GATA zinc finger domain containing, Zinc fingers C2H2-type, phosphatase 1 regulatory subunits, Interferon regulatory factors, Kruppel like factors, Notch receptors, MicroRNA protein coding host genes, NF-kappa B complex subunits, IPT domain containing, Basic helix-loop-helix proteins, and BTB domain containing. In some embodiments, the one or more transcription factors are selected from the group of transcription factor families consisting of Zinc fingers C2H2-type, BAF complex, Ring finger proteins, Forkhead boxes, GATA zinc finger domain containing, Kruppel like factors, Nuclear factors of activated T-cells, IPT domain containing, MicroRNA protein coding host genes, Notch receptors, NF-kappa B complex subunits, IPT domain containing, CUT class homeoboxes and pseudogenes, T-box transcription factors, Basic helix-loop-helix proteins, TCF/LEF transcription factor family, and Wnt enhanceosome complex. In some embodiments, the one or more transcription factors are selected from the group of transcription factor families consisting of Forkhead boxes, GATA zinc finger domain containing, Zinc fingers C2H2-type, Kruppel like factors, TCF/LEF transcription factor family, Wnt enhanceosome complex, Notch receptors, MicroRNA protein coding host genes, RAR related orphan receptors, NF-kappa B complex subunits, IPT domain containing, Runt-related transcription factors, Basic helix-loop-helix proteins. In some embodiments, the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, Notch receptors, MicroRNA protein coding host genes, Runt-related transcription factors, Basic helix-loop-helix proteins, TCF/LEF transcription factor family, Wnt enhanceosome complex, BTB domain containing. In some embodiments, the one or more transcription factors are selected from the group of transcription factor families consisting of Ring finger proteins, Forkhead boxes, Zinc fingers C2H2-type Protein, phosphatase 1 regulatory subunits, Interferon regulatory factors, Nuclear factors of activated T-cells, IPT domain containing, MicroRNA protein coding host genes, Notch receptors, MicroRNA protein coding host genes, RAR related orphan receptors, NF-kappa B complex subunits, IPT domain containing, CUT class homeoboxes and pseudogenes, Basic helix-loop-helix proteins. In some embodiments, the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, IPT domain containing, T-box transcription factors, TCF/LEF transcription factor family, Wnt enhanceosome complex, BTB domain containing, Zinc fingers C2H2-type. In some embodiments, the one or more transcription factors are selected from the group of transcription factor families consisting of Ring finger proteins, Forkhead boxes, GATA zinc finger domain containing, Interferon regulatory factors, Zinc fingers C2H2-type, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, CUT class homeoboxes and pseudogenes, Basic helix-loop-helix proteins, TCF/LEF transcription factor family, Wnt enhanceosome complex, BTB domain containing. In some embodiments, the one or more transcription factors are selected from the group of transcription factor families consisting of Zinc fingers C2H2-type, BAF complex, Forkhead boxes, Zinc fingers C2H2-type Protein, phosphatase 1 regulatory subunits, Kruppel like factors, TCF/LEF transcription factor family, Wnt enhanceosome complex, Nuclear factors of activated T-cells, IPT domain containing, Notch receptors, MicroRNA protein coding host genes, and T-box transcription factors. In some embodiments, the one or more transcription factors are selected from the group of transcription factor families consisting of Forkhead boxes, GATA zinc finger domain containing, Zinc fingers C2H2-type, TCF/LEF transcription factor family, Wnt enhanceosome complex, Nuclear factors of activated T-cells, IPT domain containing, MicroRNA protein coding host genes, Notch receptors, RAR related orphan receptors, and Basic helix-loop-helix proteins. In some embodiments, the one or more transcription factors are selected from the group of transcription factor families consisting of Forkhead boxes, GATA zinc finger domain containing, Zinc fingers C2H2-type, phosphatase 1 regulatory subunits, Kruppel like factors, TCF/LEF transcription factor family, Wnt enhanceosome complex, Nuclear factors of activated T-cells, IPT domain containing, MicroRNA protein coding host genes, Runt-related transcription factors, CUT class homeoboxes and pseudogenes, and T-box transcription factors, Basic helix-loop-helix proteins. In some embodiments, the one or more transcription factors are selected from the group of transcription factor families consisting of Zinc fingers C2H2-type, BAF complex, Forkhead boxes, GATA zinc finger domain containing, phosphatase 1 regulatory subunits, Interferon regulatory factors, Kruppel like factors, Nuclear factors of activated T-cells, IPT domain containing, MicroRNA protein coding host genes, NF-kappa B complex subunits, Runt-related transcription factors, CUT class homeoboxes and pseudogenes, and BTB domain containing. In some embodiments, the one or more transcription factors are selected from the group of transcription factor families consisting of Ring finger proteins, Forkhead boxes, GATA zinc finger domain containing, Zinc fingers C2H2-type Protein, phosphatase 1 regulatory subunits, TCF/LEF transcription factor family, Wnt enhanceosome complex, Nuclear factors of activated T-cells, IPT domain containing, MicroRNA protein coding host genes, RAR related orphan receptors, Runt-related transcription factors, T-box transcription factors, Basic helix-loop-helix proteins, and BTB domain containing. In some embodiments, the one or more transcription factors are selected from the group of transcription factor families consisting of Forkhead boxes, GATA zinc finger domain containing, Zinc fingers C2H2-type Protein, phosphatase 1 regulatory subunits, Kruppel like factors, Notch receptors, MicroRNA protein coding host genes, NF-kappa B complex subunits, IPT domain containing, and Basic helix-loop-helix proteins. In some embodiments, the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, MicroRNA protein coding host genes, Notch receptors, Runt-related transcription factors, Basic helix-loop-helix proteins, TCF/LEF transcription factor family, Wnt enhanceosome complex, and BTB domain containing. In some embodiments, the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, T-box transcription factors, TCF/LEF transcription factor family, Wnt enhanceosome complex, and BTB domain containing. In some embodiments, the one or more transcription factors comprise any one of the transcription factor combinations in Tables 1, 2, 3, 5, 6, 7, 8, 9, 10, or 11. In some embodiments, the PSC is provided in a media. In some embodiments, the media is not altered during the differentiation of the PSC into the immune cell. In some embodiments, the nucleic acid comprising an open reading frame encoding one or more transcription factors, the one or more transcription factors, or the activator of transcription of the open reading frame encoding one or more transcription factors induces differentiation of the PSC into the immune cell in 11 days or less, 5 days or less, 4 days or less, 1 day or less.

Another aspect of the present disclosure provides a population of cells comprising two or more of the immune cell as disclosed herein. In some embodiments, the cells are adherent cells. In some embodiments, the cells are suspension cells. In some embodiments, at least 5% of the cells express FOXP3. In some embodiments, at least 10% of the cells express FOXP3. In some embodiments, at least 20% of the cells express FOXP3. In some embodiments, at least 30% of the cells express FOXP3. In some embodiments, at least 40% of the cells express FOXP3. In some embodiments, at least 1% of the cells express CD45. In some embodiments, at least 3% of the cells express CD45. In some embodiments, at least 3% of the cells express CD45. In some embodiments, at least 4% of the cells express CD45. In some embodiments, at least 5% of the cells express CD45. In some embodiments, at least 6% of the cells express CD45. In some embodiments, at least 1% of the cells express CD34. In some embodiments, at least 2% of the cells express CD34. In some embodiments, at least 2.5% of the cells express CD34. In some embodiments, at least 2% of the cells express CD4. In some embodiments, at least 3% of the cells express CD4. In some embodiments, at least 4% of the cells express CD4. In some embodiments, at least 4% of the cells express CD4. In some embodiments, no nutrient, growth factor or microenvironmental/matrix optimizations are performed.

In another aspect, the present disclosure provides an immune cell comprising a nucleic acid comprising an open reading frame encoding one or more transcription factors, one or more transcription factors, or an activator of transcription of the open reading frame encoding one or more transcription factors, wherein the nucleic acid, wherein the immune cell is provided in a media, and wherein the media is not altered during the differentiation of the PSC into the immune cell. In some embodiments, the immune cell expresses FOXP3. In some embodiments, the immune cell expresses CD34. In some embodiments, the immune cell expresses CD45. In some embodiments, the immune cell expresses CD4. In some embodiments, the immune cell is a regulatory T cell. In some embodiments, the immune cell is a hematopoietic progenitor. In some embodiments, the immune cell is a leukocyte. In some embodiments, the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, two or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, three or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, four or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the nucleic acid comprises two or more open reading frame encoding one or more transcription factors. In some embodiments, the one or more transcription factors are selected from the group of transcription factor families consisting of Zinc fingers C2H2-type, BAF complex, Runt-related transcription factors, BTB domain containing, basic helix-loop-helix (bHLH), Nuclear factor-KB, and basic helix-loop-helix. In some embodiments, the one or more transcription factors are selected from the group of transcription factor families consisting of Zinc fingers C2H2-type, Ring finger proteins, Forkhead boxes, Notch receptors, MicroRNA protein coding host genes, GATA zinc finger domain containing, Nuclear factors of activated T-cells, IPT domain containing, TCF/LEF transcription factor family, Wnt enhanceosome complex, NF-kappa B complex subunits, IPT domain containing, Runt-related transcription factors, CUT class homeoboxes and pseudogenes, and BTB domain containing. In some embodiments, the one or more transcription factors are selected from the group of transcription factor families consisting of Zinc fingers C2H2-type, BAF complex, Ring finger proteins, Forkhead boxes, GATA zinc finger domain containing, Nuclear factors of activated T-cells, IPT domain containing, phosphatase 1 regulatory subunits, TCF/LEF transcription factor family, Wnt enhanceosome complex, Notch receptors, MicroRNA protein coding host genes, RAR related orphan receptors, CUT class homeoboxes and pseudogenes, T-box transcription factors, Basic helix-loop-helix proteins, and BTB domain containing. In some embodiments, the one or more transcription factors are selected from the group of transcription factor families consisting of Forkhead boxes, GATA zinc finger domain containing, Zinc fingers C2H2-type, phosphatase 1 regulatory subunits, Interferon regulatory factors, Kruppel like factors, Notch receptors, MicroRNA protein coding host genes, NF-kappa B complex subunits, IPT domain containing, Basic helix-loop-helix proteins, and BTB domain containing. In some embodiments, the one or more transcription factors are selected from the group of transcription factor families consisting of Zinc fingers C2H2-type, BAF complex, Ring finger proteins, Forkhead boxes, GATA zinc finger domain containing, Kruppel like factors, Nuclear factors of activated T-cells, IPT domain containing, MicroRNA protein coding host genes, Notch receptors, NF-kappa B complex subunits, IPT domain containing, CUT class homeoboxes and pseudogenes, T-box transcription factors, Basic helix-loop-helix proteins, TCF/LEF transcription factor family, and Wnt enhanceosome complex. In some embodiments, the one or more transcription factors are selected from the group of transcription factor families consisting of Forkhead boxes, GATA zinc finger domain containing, Zinc fingers C2H2-type, Kruppel like factors, TCF/LEF transcription factor family, Wnt enhanceosome complex, Notch receptors, MicroRNA protein coding host genes, RAR related orphan receptors, NF-kappa B complex subunits, IPT domain containing, Runt-related transcription factors, Basic helix-loop-helix proteins. In some embodiments, the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, Notch receptors, MicroRNA protein coding host genes, Runt-related transcription factors, Basic helix-loop-helix proteins, TCF/LEF transcription factor family, Wnt enhanceosome complex, BTB domain containing. In some embodiments, the one or more transcription factors are selected from the group of transcription factor families consisting of Ring finger proteins, Forkhead boxes, Zinc fingers C2H2-type Protein, phosphatase 1 regulatory subunits, Interferon regulatory factors, Nuclear factors of activated T-cells, IPT domain containing, MicroRNA protein coding host genes, Notch receptors, MicroRNA protein coding host genes, RAR related orphan receptors, NF-kappa B complex subunits, IPT domain containing, CUT class homeoboxes and pseudogenes, Basic helix-loop-helix proteins. In some embodiments, the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, IPT domain containing, T-box transcription factors, TCF/LEF transcription factor family, Wnt enhanceosome complex, BTB domain containing, Zinc fingers C2H2-type. In some embodiments, the one or more transcription factors are selected from the group of transcription factor families consisting of Ring finger proteins, Forkhead boxes, GATA zinc finger domain containing, Interferon regulatory factors, Zinc fingers C2H2-type, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, CUT class homeoboxes and pseudogenes, Basic helix-loop-helix proteins, TCF/LEF transcription factor family, Wnt enhanceosome complex, BTB domain containing. In some embodiments, the one or more transcription factors are selected from the group of transcription factor families consisting of Zinc fingers C2H2-type, BAF complex, Forkhead boxes, Zinc fingers C2H2-type Protein, phosphatase 1 regulatory subunits, Kruppel like factors, TCF/LEF transcription factor family, Wnt enhanceosome complex, Nuclear factors of activated T-cells, IPT domain containing, Notch receptors, MicroRNA protein coding host genes, and T-box transcription factors. In some embodiments, the one or more transcription factors are selected from the group of transcription factor families consisting of Forkhead boxes, GATA zinc finger domain containing, Zinc fingers C2H2-type, TCF/LEF transcription factor family, Wnt enhanceosome complex, Nuclear factors of activated T-cells, IPT domain containing, MicroRNA protein coding host genes, Notch receptors, RAR related orphan receptors, and Basic helix-loop-helix proteins. In some embodiments, the one or more transcription factors are selected from the group of transcription factor families consisting of Forkhead boxes, GATA zinc finger domain containing, Zinc fingers C2H2-type, phosphatase 1 regulatory subunits, Kruppel like factors, TCF/LEF transcription factor family, Wnt enhanceosome complex, Nuclear factors of activated T-cells, IPT domain containing, MicroRNA protein coding host genes, Runt-related transcription factors, CUT class homeoboxes and pseudogenes, and T-box transcription factors, Basic helix-loop-helix proteins. In some embodiments, the one or more transcription factors are selected from the group of transcription factor families consisting of Zinc fingers C2H2-type, BAF complex, Forkhead boxes, GATA zinc finger domain containing, phosphatase 1 regulatory subunits, Interferon regulatory factors, Kruppel like factors, Nuclear factors of activated T-cells, IPT domain containing, MicroRNA protein coding host genes, NF-kappa B complex subunits, Runt-related transcription factors, CUT class homeoboxes and pseudogenes, and BTB domain containing. In some embodiments, the one or more transcription factors are selected from the group of transcription factor families consisting of Ring finger proteins, Forkhead boxes, GATA zinc finger domain containing, Zinc fingers C2H2-type Protein, phosphatase 1 regulatory subunits, TCF/LEF transcription factor family, Wnt enhanceosome complex, Nuclear factors of activated T-cells, IPT domain containing, MicroRNA protein coding host genes, RAR related orphan receptors, Runt-related transcription factors, T-box transcription factors, Basic helix-loop-helix proteins, and BTB domain containing. In some embodiments, the one or more transcription factors are selected from the group of transcription factor families consisting of Forkhead boxes, GATA zinc finger domain containing, Zinc fingers C2H2-type Protein, phosphatase 1 regulatory subunits, Kruppel like factors, Notch receptors, MicroRNA protein coding host genes, NF-kappa B complex subunits, IPT domain containing, and Basic helix-loop-helix proteins. In some embodiments, the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, MicroRNA protein coding host genes, Notch receptors, Runt-related transcription factors, Basic helix-loop-helix proteins, TCF/LEF transcription factor family, Wnt enhanceosome complex, and BTB domain containing. In some embodiments, the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, T-box transcription factors, TCF/LEF transcription factor family, Wnt enhanceosome complex, and BTB domain containing. In some embodiments, the one or more transcription factors comprise any one of the transcription factor combinations in Tables 1, 2, 3, 5, 6, 7, 8, 9, 10, or 11. In some embodiments, the nucleic acid comprising an open reading frame encoding one or more transcription factors, the one or more transcription factors, or the activator of transcription of the open reading frame encoding one or more transcription factors induces differentiation of the PSC into the immune cell in 28 days or less. In some embodiments, the nucleic acid comprising an open reading frame encoding one or more transcription factors, the one or more transcription factors, or the activator of transcription of the open reading frame encoding one or more transcription factors induces differentiation of the PSC into the immune cell in 11 days or less, 5 days or less, 4 days or less, 1 day or less.

Another aspect of the present disclosure provides a population of cells comprising two or more of the immune cell as disclosed herein. In some embodiments, the cells are adherent cells. In some embodiments, the cells are suspension cells. In some embodiments, at least 5% of the cells express FOXP3. In some embodiments, at least 10% of the cells express FOXP3. In some embodiments, at least 20% of the cells express FOXP3. In some embodiments, at least 30% of the cells express FOXP3. In some embodiments, at least 40% of the cells express FOXP3. In some embodiments, at least 1% of the cells express CD45. In some embodiments, at least 3% of the cells express CD45. In some embodiments, at least 3% of the cells express CD45. In some embodiments, at least 4% of the cells express CD45. In some embodiments, at least 5% of the cells express CD45. In some embodiments, at least 6% of the cells express CD45. In some embodiments, at least 1% of the cells express CD34. In some embodiments, at least 2% of the cells express CD34. In some embodiments, at least 2.5% of the cells express CD34. In some embodiments, at least 2% of the cells express CD4. In some embodiments, at least 3% of the cells express CD4. In some embodiments, at least 4% of the cells express CD4. In some embodiments, at least 4% of the cells express CD4. In some embodiments, no nutrient, growth factor or microenvironmental/matrix optimizations are performed.

Another aspect of the present disclosure provides a FOXP3-expressing cell comprising a nucleic acid comprising an open reading frame encoding one or more transcription factors, one or more transcription factors, or an activator of transcription of the open reading frame encoding one or more transcription factors, wherein the nucleic acid comprising the open reading frame encoding the one or more transcription factors, the one or more transcription factors, or the activator of transcription of the open reading frame encoding one or more transcription factors induces the differentiation of the FOXP3-expressing cell from the PSC in 28 days or less. In some embodiments, the FOXP3-expressing cell further expresses CD34. In some embodiments, the FOXP3-expressing cell further expresses CD45. In some embodiments, the FOXP3-expressing cell further expresses CD4. In some embodiments, the FOXP3-expressing cell is a regulatory T cell. In some embodiments, the FOXP3-expressing cell is a hematopoietic progenitor. In some embodiments, the FOXP3-expressing cell is a leukocyte. In some embodiments, the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors comprise any one of the transcription factor combinations in Tables 1, 2, 3, 5, 6, 7, 8, 9, 10, or 11. In some embodiments, the nucleic acid comprising an open reading frame encoding one or more transcription factors, the one or more transcription factors, or the activator of transcription of the open reading frame encoding one or more transcription factors induces differentiation of the PSC into the FOXP3-expressing cell in 28 days or less. In some embodiments, the PSC is provided in a media. In some embodiments, the media is not altered during the differentiation of the PSC into the FOXP3-expressing cell.

Another aspect of the present disclosure provides a population of cells comprising two or more of the FOXP3-expressing cell as disclosed herein. In some embodiments, the cells are adherent cells. In some embodiments, the cells are suspension cells. In some embodiments, at least 5% of the cells express FOXP3. In some embodiments, at least 10% of the cells express FOXP3. In some embodiments, at least 20% of the cells express FOXP3. In some embodiments, at least 30% of the cells express FOXP3. In some embodiments, at least 40% of the cells express FOXP3. In some embodiments, no nutrient, growth factor or microenvironmental/matrix optimizations are performed.

Another aspect of the present disclosure provides a FOXP3-expressing cell comprising a nucleic acid comprising an open reading frame encoding one or more transcription factors, one or more transcription factors, or an activator of transcription of the open reading frame encoding one or more transcription factors, wherein the nucleic acid comprising an open reading frame encoding one or more transcription factors, the one or more transcription factors, or the activator of transcription of the open reading frame encoding one or more transcription factors induce the differentiation of the FOXP3-expressing cell from a pluripotent stem cell (PSC), and wherein the PSC is provided in a media that is not altered during the differentiation of the PSC into the FOXP3-expressing cell. In some embodiments, the FOXP3-expressing cell further expresses CD34. In some embodiments, the FOXP3-expressing cell further expresses CD45. In some embodiments, the FOXP3-expressing cell further expresses CD4. In some embodiments, the FOXP3-expressing cell is a regulatory T cell. In some embodiments, the FOXP3-expressing cell is a hematopoietic progenitor. In some embodiments, the FOXP3-expressing cell is a leukocyte. In some embodiments, the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors comprise any one of the transcription factor combinations in Tables 1, 2, 3, 5, 6, 7, 8, 9, 10, or 11. In some embodiments, the nucleic acid comprising an open reading frame encoding one or more transcription factors, the one or more transcription factors, or the activator of transcription of the open reading frame encoding one or more transcription factors induce the differentiation of the FOXP3-expressing cell from the PSC in 28 days or less. In some embodiments, the PSC is provided in a media. In some embodiments, the media is not altered during the differentiation of the PSC into the FOXP3-expressing cell. In some embodiments, the cells are adherent cells. In some embodiments, the cells are suspension cells. In some embodiments, at least 5% of the cells express FOXP3. In some embodiments, at least 10% of the cells express FOXP3. In some embodiments, at least 20% of the cells express FOXP3. In some embodiments, at least 30% of the cells express FOXP3. In some embodiments, at least 40% of the cells express FOXP3. In some embodiments, no nutrient, growth factor or microenvironmental/matrix optimizations are performed.

Another aspect of the present disclosure provides a CD45-expressing cell comprising a nucleic acid comprising an open reading frame encoding one or more transcription factors, one or more transcription factors, or an activator of transcription of the open reading frame encoding one or more transcription factors, wherein the nucleic acid comprising the open reading frame encoding the one or more transcription factors, the one or more transcription factors, or the activator of transcription of the open reading frame encoding one or more transcription factors induces the differentiation of the CD45-expressing cell from the PSC in 28 days or less. In some embodiments, the CD45-expressing cell further expresses CD34. In some embodiments, the CD45-expressing cell further expresses CD45. In some embodiments, the CD45-expressing cell further expresses CD4. In some embodiments, the CD45-expressing cell is a regulatory T cell. In some embodiments, the CD45-expressing cell is a hematopoietic progenitor. In some embodiments, the CD45-expressing cell is a leukocyte. In some embodiments, the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors comprise any one of the transcription factor combinations in Tables 1, 2, 3, 5, 6, 7, 8, 9, 10, or 11. In some embodiments, the nucleic acid comprising an open reading frame encoding one or more transcription factors, the one or more transcription factors, or the activator of transcription of the open reading frame encoding one or more transcription factors induces differentiation of the PSC into the CD45-expressing cell in 11 days or less, 5 days or less, 4 days or less, 1 day or less. In some embodiments, the PSC is provided in a media. In some embodiments, the media is not altered during the differentiation of the PSC into the FOXP3-expressing cell.

Another aspect of the present disclosure provides a population of cells comprising two or more of the CD45-expressing cell as disclosed herein. In some embodiments, the cells are adherent cells. In some embodiments, the cells are suspension cells. In some embodiments, at least 1% of the cells express CD45. In some embodiments, at least 3% of the cells express CD45. In some embodiments, at least 3% of the cells express CD45. In some embodiments, at least 4% of the cells express CD45. In some embodiments, at least 5% of the cells express CD45. In some embodiments, at least 6% of the cells express CD45. In some embodiments, no nutrient, growth factor or microenvironmental/matrix optimizations are performed.

Another aspect of the present disclosure provides a CD45-expressing cell comprising a nucleic acid comprising an open reading frame encoding one or more transcription factors, one or more transcription factors, or an activator of transcription of the open reading frame encoding one or more transcription factors, wherein the nucleic acid comprising an open reading frame encoding one or more transcription factors, the one or more transcription factors, or the activator of transcription of the open reading frame encoding one or more transcription factors induce the differentiation of the CD45-expressing cell from a pluripotent stem cell (PSC), and wherein the PSC is provided in a media that is not altered during the differentiation of the PSC into the CD45-expressing cell. In some embodiments, the CD45-expressing cell further expresses CD34. In some embodiments, the CD45-expressing cell further expresses CD45. In some embodiments, the CD45-expressing cell further expresses CD4. In some embodiments, the CD45-expressing cell is a regulatory T cell. In some embodiments, the CD45-expressing cell is a NK cell. In some embodiments, the CD45-expressing cell is a hematopoietic progenitor. In some embodiments, the CD45-expressing cell is a leukocyte. In some embodiments, the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors comprise any one of the transcription factor combinations in Tables 1, 2, 3, 5, 6, 7, 8, 9, 10, or 11. In some embodiments, the nucleic acid comprising an open reading frame encoding one or more transcription factors, the one or more transcription factors, or the activator of transcription of the open reading frame encoding one or more transcription factors induces differentiation of the PSC into the CD45-expressing cell in 28 days or less. In some embodiments, the nucleic acid comprising an open reading frame encoding one or more transcription factors, the one or more transcription factors, or the activator of transcription of the open reading frame encoding one or more transcription factors induce the differentiation of the CD45-expressing cell from the PSC in 11 days or less, 5 days or less, 4 days or less, 1 day or less.

In another aspect, the present disclosure provides a population of cells comprising two or more of the CD45-expressing cell as disclosed herein. In some embodiments, the cells are adherent cells. In some embodiments, the cells are suspension cells. In some embodiments, at least 1% of the cells express CD45. In some embodiments, at least 3% of the cells express CD45. In some embodiments, at least 3% of the cells express CD45. In some embodiments, at least 4% of the cells express CD45. In some embodiments, at least 5% of the cells express CD45. In some embodiments, at least 6% of the cells express CD45. In some embodiments, no nutrient, growth factor or microenvironmental/matrix optimizations are performed.

Another aspect of the present disclosure provides a CD34-expressing cell comprising a nucleic acid comprising an open reading frame encoding one or more transcription factors, one or more transcription factors, or an activator of transcription of the open reading frame encoding one or more transcription factors, wherein the nucleic acid comprising the open reading frame encoding the one or more transcription factors, the one or more transcription factors, or the activator of transcription of the open reading frame encoding one or more transcription factors induces the differentiation of the CD34-expressing cell from the PSC in 28 days or less. In some embodiments, the CD34-expressing cell further expresses FOXP3. In some embodiments, the CD34-expressing cell further expresses CD45. In some embodiments, the CD34-expressing cell further expresses CD4. In some embodiments, the CD34-expressing cell is a regulatory T cell. In some embodiments, the CD34-expressing cell is a hematopoietic progenitor. In some embodiments, the CD34-expressing cell is a leukocyte. In some embodiments, the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors comprise any one of the transcription factor combinations in Tables 1, 2, 3, 5, 6, 7, 8, 9, 10, or 11. In some embodiments, the nucleic acid comprising an open reading frame encoding one or more transcription factors, the one or more transcription factors, or the activator of transcription of the open reading frame encoding one or more transcription factors induces differentiation of the PSC into the CD34-expressing cell in 28 days or less. In some embodiments, the PSC is provided in a media. In some embodiments, the media is not altered during the differentiation of the PSC into the FOXP3-expressing cell.

Another aspect of the present disclosure provides a population of cells comprising two or more of the CD34-expressing cell as disclosed herein. In some embodiments, the cells are adherent cells. In some embodiments, the cells are suspension cells. In some embodiments, at least 1% of the cells express CD34. In some embodiments, at least 2% of the cells express CD34. In some embodiments, at least 2.5% of the cells express CD34. In some embodiments, no nutrient, growth factor or microenvironmental/matrix optimizations are performed.

Another aspect of the present disclosure provides a CD34-expressing cell comprising a nucleic acid comprising an open reading frame encoding one or more transcription factors, one or more transcription factors, or an activator of transcription of the open reading frame encoding one or more transcription factors, wherein the nucleic acid comprising an open reading frame encoding one or more transcription factors, the one or more transcription factors, or the activator of transcription of the open reading frame encoding one or more transcription factors induce the differentiation of the CD34-expressing cell from a pluripotent stem cell (PSC), and wherein the PSC is provided in a media that is not altered during the differentiation of the PSC into the CD34-expressing cell. In some embodiments, the CD34-expressing cell further expresses FOXP3. In some embodiments, the CD34-expressing cell further expresses CD45. In some embodiments, the CD34-expressing cell further expresses CD4. In some embodiments, the CD34-expressing cell is a regulatory T cell. In some embodiments, the CD34-expressing cell is a hematopoietic progenitor. In some embodiments, the CD34-expressing cell is a leukocyte. In some embodiments, the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors comprise any one of the transcription factor combinations in Tables 1, 2, 3, 5, 6, 7, 8, 9, 10, or 11. In some embodiments, the nucleic acid comprising an open reading frame encoding one or more transcription factors, the one or more transcription factors, or the activator of transcription of the open reading frame encoding one or more transcription factors induces differentiation of the PSC into the CD34-expressing cell in 28 days or less. In some embodiments, the nucleic acid comprising an open reading frame encoding one or more transcription factors, the one or more transcription factors, or the activator of transcription of the open reading frame encoding one or more transcription factors induce the differentiation of the CD34-expressing cell from the PSC in 11 days or less, 5 days or less, 4 days or less, 1 day or less. In some embodiments, the nucleic acid comprising an open reading frame encoding one or more transcription factors, the one or more transcription factors, or the activator of transcription of the open reading frame encoding one or more transcription factors induce the differentiation of the CD34-expressing cell from the PSC in 4 days or less.

Another aspect of the present disclosure provides a population of cells comprising two or more of the CD34-expressing cell as disclosed herein. In some embodiments, the cells are adherent cells. In some embodiments, the cells are suspension cells. In some embodiments, at least 1% of the cells express CD34. In some embodiments, at least 2% of the cells express CD34. In some embodiments, at least 2.5% of the cells express CD34. In some embodiments, no nutrient, growth factor or microenvironmental/matrix optimizations are performed.

Another aspect of the present disclosure provides a CD4-expressing cell comprising a nucleic acid comprising an open reading frame encoding one or more transcription factors, one or more transcription factors, or an activator of transcription of the open reading frame encoding one or more transcription factors, wherein the nucleic acid comprising the open reading frame encoding the one or more transcription factors, the one or more transcription factors, or the activator of transcription of the open reading frame encoding one or more transcription factors induces the differentiation of the CD4-expressing cell from the PSC in 28 days or less. In some embodiments, the CD4-expressing cell further expresses FOXP3. In some embodiments, the CD4-expressing cell further expresses CD45. In some embodiments, the CD4-expressing cell is a regulatory T cell. In some embodiments, the CD4-expressing cell is a hematopoietic progenitor. In some embodiments, the CD4-expressing cell is a leukocyte. In some embodiments, the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors comprise any one of the transcription factor combinations in Tables 1, 2, 3, 5, 6, 7, 8, 9, 10, or 11. In some embodiments, the nucleic acid comprising an open reading frame encoding one or more transcription factors, the one or more transcription factors, or the activator of transcription of the open reading frame encoding one or more transcription factors induces differentiation of the PSC into the CD4-expressing cell in 28 days or less. In some embodiments, the PSC is provided in a media. In some embodiments, the media is not altered during the differentiation of the PSC into the FOXP3-expressing cell.

Another aspect of the present disclosure provides a population of cells comprising two or more of the CD4-expressing cell as disclosed herein. In some embodiments, the cells are adherent cells. In some embodiments, the cells are suspension cells. In some embodiments, at least 2% of the cells express CD4. In some embodiments, at least 3% of the cells express CD4. In some embodiments, at least 4% of the cells express CD4. In some embodiments, at least 4% of the cells express CD4. In some embodiments, no nutrient, growth factor or microenvironmental/matrix optimizations are performed.

Another aspect of the present disclosure provides a CD4-expressing cell comprising a nucleic acid comprising an open reading frame encoding one or more transcription factors, one or more transcription factors, or an activator of transcription of the open reading frame encoding one or more transcription factors, wherein the nucleic acid comprising an open reading frame encoding one or more transcription factors, the one or more transcription factors, or the activator of transcription of the open reading frame encoding one or more transcription factors induce the differentiation of the CD4-expressing cell from a pluripotent stem cell (PSC), and wherein the PSC is provided in a media that is not altered during the differentiation of the PSC into the CD4-expressing cell. In some embodiments, the CD4-expressing cell further expresses FOXP3. In some embodiments, the CD4-expressing cell further expresses CD45. In some embodiments, the CD4-expressing cell further expresses CD4. In some embodiments, the CD4-expressing cell is a regulatory T cell. In some embodiments, the CD4-expressing cell is a hematopoietic progenitor. In some embodiments, the CD4-expressing cell is a leukocyte. In some embodiments, the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors comprise any one of the transcription factor combinations in Tables 1, 2, 3, 5, 6, 7, 8, 9, 10, or 11. In some embodiments, the nucleic acid comprising an open reading frame encoding one or more transcription factors, the one or more transcription factors, or the activator of transcription of the open reading frame encoding one or more transcription factors induces differentiation of the PSC into the CD4-expressing cell in 28 days or less. In some embodiments, the nucleic acid comprising an open reading frame encoding one or more transcription factors, the one or more transcription factors, or the activator of transcription of the open reading frame encoding one or more transcription factors induce the differentiation of the CD4-expressing cell from the PSC in 11 days or less. In some embodiments, the nucleic acid comprising an open reading frame encoding one or more transcription factors, the one or more transcription factors, or the activator of transcription of the open reading frame encoding one or more transcription factors induce the differentiation of the CD4-expressing cell from the PSC in 4 days or less, or 1 day or less.

Another aspect of the present disclosure provides a population of cells comprising two or more of the CD4-expressing cell as disclosed herein. In some embodiments, the cells are adherent cells. In some embodiments, the cells are suspension cells. In some embodiments, at least 2% of the cells express CD4. In some embodiments, at least 3% of the cells express CD4. In some embodiments, at least 4% of the cells express CD4. In some embodiments, at least 4% of the cells express CD4. In some embodiments, no nutrient, growth factor or microenvironmental/matrix optimizations are performed.

Another aspect of the present disclosure provides a pluripotent stem cell (PSC) comprising a nucleic acid comprising an open reading frame encoding one or more transcription factors, one or more transcription factors, or an activator of transcription of the open reading frame encoding one or more transcription factors, wherein the one or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, wherein the nucleic acid comprising an open reading frame encoding one or more transcription factors, the one or more transcription factors, or the activator of transcription of the open reading frame encoding one or more transcription factors induces the differentiation of the PSC into an immune cell. In some embodiments, wherein the immune cell expresses FOXP3. In some embodiments, wherein the immune cell expresses CD34. In some embodiments, wherein the immune cell expresses CD45. In some embodiments, wherein the immune cell expresses CD4. In some embodiments, wherein the immune cell is a regulatory T cell. In some embodiments, wherein the immune cell is a hematopoietic progenitor. In some embodiments, wherein the immune cell is a leukocyte. In some embodiments, wherein the nucleic acid comprises two or more open reading frame encoding one or more transcription factors. In some embodiments, wherein the nucleic acid comprises three or more open reading frame encoding one or more transcription factors. In some embodiments, wherein the nucleic acid comprises four or more open reading frame encoding one or more transcription factors. In some embodiments, wherein two or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, wherein three or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, wherein four or more transcription factors are selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, wherein the one or more transcription factors are selected from the group of transcription factor families consisting of Zinc fingers C2H2-type, BAF complex, Runt-related transcription factors, BTB domain containing, basic helix-loop-helix (bHLH), Nuclear factor-KB, and basic helix-loop-helix. In some embodiments, wherein the one or more transcription factors are selected from the group of transcription factor families consisting of Zinc fingers C2H2-type, Ring finger proteins, Forkhead boxes, Notch receptors, MicroRNA protein coding host genes, GATA zinc finger domain containing, Nuclear factors of activated T-cells, IPT domain containing, TCF/LEF transcription factor family, Wnt enhanceosome complex, NF-kappa B complex subunits, IPT domain containing, Runt-related transcription factors, CUT class homeoboxes and pseudogenes, and BTB domain containing. In some embodiments, wherein the one or more transcription factors are selected from the group of transcription factor families consisting of Zinc fingers C2H2-type, BAF complex, Ring finger proteins, Forkhead boxes, GATA zinc finger domain containing, Nuclear factors of activated T-cells, IPT domain containing, phosphatase 1 regulatory subunits, TCF/LEF transcription factor family, Wnt enhanceosome complex, Notch receptors, MicroRNA protein coding host genes, RAR related orphan receptors, CUT class homeoboxes and pseudogenes, T-box transcription factors, Basic helix-loop-helix proteins, and BTB domain containing. In some embodiments, wherein the one or more transcription factors are selected from the group of transcription factor families consisting of Forkhead boxes, GATA zinc finger domain containing, Zinc fingers C2H2-type, phosphatase 1 regulatory subunits, Interferon regulatory factors, Kruppel like factors, Notch receptors, MicroRNA protein coding host genes, NF-kappa B complex subunits, IPT domain containing, Basic helix-loop-helix proteins, and BTB domain containing. In some embodiments, wherein the one or more transcription factors are selected from the group of transcription factor families consisting of Zinc fingers C2H2-type, BAF complex, Ring finger proteins, Forkhead boxes, GATA zinc finger domain containing, Kruppel like factors, Nuclear factors of activated T-cells, IPT domain containing, MicroRNA protein coding host genes, Notch receptors, NF-kappa B complex subunits, IPT domain containing, CUT class homeoboxes and pseudogenes, T-box transcription factors, Basic helix-loop-helix proteins, TCF/LEF transcription factor family, and Wnt enhanceosome complex. In some embodiments, wherein the one or more transcription factors are selected from the group of transcription factor families consisting of Forkhead boxes, GATA zinc finger domain containing, Zinc fingers C2H2-type, Kruppel like factors, TCF/LEF transcription factor family, Wnt enhanceosome complex, Notch receptors, MicroRNA protein coding host genes, RAR related orphan receptors, NF-kappa B complex subunits, IPT domain containing, Runt-related transcription factors, Basic helix-loop-helix proteins. In some embodiments, wherein the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, Notch receptors, MicroRNA protein coding host genes, Runt-related transcription factors, Basic helix-loop-helix proteins, TCF/LEF transcription factor family, Wnt enhanceosome complex, BTB domain containing. In some embodiments, wherein the one or more transcription factors are selected from the group of transcription factor families consisting of Ring finger proteins, Forkhead boxes, Zinc fingers C2H2-type Protein, phosphatase 1 regulatory subunits, Interferon regulatory factors, Nuclear factors of activated T-cells, IPT domain containing, MicroRNA protein coding host genes, Notch receptors, MicroRNA protein coding host genes, RAR related orphan receptors, NF-kappa B complex subunits, IPT domain containing, CUT class homeoboxes and pseudogenes, Basic helix-loop-helix proteins. In some embodiments, wherein the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, IPT domain containing, T-box transcription factors, TCF/LEF transcription factor family, Wnt enhanceosome complex, BTB domain containing, Zinc fingers C2H2-type. In some embodiments, wherein the one or more transcription factors are selected from the group of transcription factor families consisting of Ring finger proteins, Forkhead boxes, GATA zinc finger domain containing, Interferon regulatory factors, Zinc fingers C2H2-type, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, CUT class homeoboxes and pseudogenes, Basic helix-loop-helix proteins, TCF/LEF transcription factor family, Wnt enhanceosome complex, BTB domain containing. In some embodiments, wherein the one or more transcription factors are selected from the group of transcription factor families consisting of Zinc fingers C2H2-type, BAF complex, Forkhead boxes, Zinc fingers C2H2-type Protein, phosphatase 1 regulatory subunits, Kruppel like factors, TCF/LEF transcription factor family, Wnt enhanceosome complex, Nuclear factors of activated T-cells, IPT domain containing, Notch receptors, MicroRNA protein coding host genes, and T-box transcription factors. In some embodiments, wherein the one or more transcription factors are selected from the group of transcription factor families consisting of Forkhead boxes, GATA zinc finger domain containing, Zinc fingers C2H2-type, TCF/LEF transcription factor family, Wnt enhanceosome complex, Nuclear factors of activated T-cells, IPT domain containing, MicroRNA protein coding host genes, Notch receptors, RAR related orphan receptors, and Basic helix-loop-helix proteins. In some embodiments, wherein the one or more transcription factors are selected from the group of transcription factor families consisting of Forkhead boxes, GATA zinc finger domain containing, Zinc fingers C2H2-type, phosphatase 1 regulatory subunits, Kruppel like factors, TCF/LEF transcription factor family, Wnt enhanceosome complex, Nuclear factors of activated T-cells, IPT domain containing, MicroRNA protein coding host genes, Runt-related transcription factors, CUT class homeoboxes and pseudogenes, and T-box transcription factors, Basic helix-loop-helix proteins. In some embodiments, wherein the one or more transcription factors are selected from the group of transcription factor families consisting of Zinc fingers C2H2-type, BAF complex, Forkhead boxes, GATA zinc finger domain containing, phosphatase 1 regulatory subunits, Interferon regulatory factors, Kruppel like factors, Nuclear factors of activated T-cells, IPT domain containing, MicroRNA protein coding host genes, NF-kappa B complex subunits, Runt-related transcription factors, CUT class homeoboxes and pseudogenes, and BTB domain containing. In some embodiments, wherein the one or more transcription factors are selected from the group of transcription factor families consisting of Ring finger proteins, Forkhead boxes, GATA zinc finger domain containing, Zinc fingers C2H2-type Protein, phosphatase 1 regulatory subunits, TCF/LEF transcription factor family, Wnt enhanceosome complex, Nuclear factors of activated T-cells, IPT domain containing, MicroRNA protein coding host genes, RAR related orphan receptors, Runt-related transcription factors, T-box transcription factors, Basic helix-loop-helix proteins, and BTB domain containing. In some embodiments, wherein the one or more transcription factors are selected from the group of transcription factor families consisting of Forkhead boxes, GATA zinc finger domain containing, Zinc fingers C2H2-type Protein, phosphatase 1 regulatory subunits, Kruppel like factors, Notch receptors, MicroRNA protein coding host genes, NF-kappa B complex subunits, IPT domain containing, and Basic helix-loop-helix proteins. In some embodiments, wherein the one or more transcription factors are selected from the group of transcription factor families consisting of Forkhead boxes, GATA zinc finger domain containing, Zinc fingers C2H2-type Protein, phosphatase 1 regulatory subunits, Nuclear factors of activated T-cells, IPT domain containing, MicroRNA protein coding host genes, Notch receptors, Runt-related transcription factors, Basic helix-loop-helix proteins, TCF/LEF transcription factor family, Wnt enhanceosome complex, and BTB domain containing. In some embodiments, wherein the one or more transcription factors are selected from the group of transcription factor families consisting of Zinc fingers C2H2-type, BAF complex, Forkhead boxes, GATA zinc finger domain containing, Nuclear factors of activated T-cells, IPT domain containing, MicroRNA protein coding host genes, Notch receptors, RAR related orphan receptors, NF-kappa B complex subunits, T-box transcription factors, TCF/LEF transcription factor family, Wnt enhanceosome complex, and BTB domain containing. In some embodiments, the one or more transcription factors comprise any one of the transcription factor combinations in Tables 1, 2, 3, 5, 6, 7, 8, 9, 10, or 11. In some embodiments, wherein the PSC is provided in a media. In some embodiments, wherein the media is not altered during the differentiation of the PSC into the immune cell. In some embodiments, wherein the nucleic acid comprising an open reading frame encoding one or more transcription factors, the one or more transcription factors, or the activator of transcription of the open reading frame encoding one or more transcription factors induces differentiation of the PSC into an immune cell in 28 days or less.

Another aspect of the present disclosure provides a population of cells comprising two or more of the immune cell as disclosed herein. In some embodiments, the cells are adherent cells. In some embodiments, the cells are suspension cells. In some embodiments, at least 5% of the cells express FOXP3. In some embodiments, at least 10% of the cells express FOXP3. In some embodiments, at least 20% of the cells express FOXP3. In some embodiments, at least 30% of the cells express FOXP3. In some embodiments, at least 40% of the cells express FOXP3. In some embodiments, at least 1% of the cells express CD45. In some embodiments, at least 3% of the cells express CD45. In some embodiments, at least 3% of the cells express CD45. In some embodiments, at least 4% of the cells express CD45. In some embodiments, at least 5% of the cells express CD45. In some embodiments, at least 6% of the cells express CD45. In some embodiments, at least 1% of the cells express CD34. In some embodiments, at least 2% of the cells express CD34. In some embodiments, at least 2.5% of the cells express CD34. In some embodiments, at least 2% of the cells express CD4. In some embodiments, at least 3% of the cells express CD4. In some embodiments, at least 4% of the cells express CD4. In some embodiments, at least 4% of the cells express CD4. In some embodiments, no nutrient, growth factor or microenvironmental/matrix optimizations are performed.

An aspect of instant disclosure is a pluripotent stem cell (PSC) comprising a nucleic acid comprising an open reading frame encoding one or more transcription factors, one or more transcription factors, or an activator of transcription of the open reading frame encoding one or more transcription factors, wherein the nucleic acid comprising an open reading frame encoding one or more transcription factors, the one or more transcription factors, or the activator of transcription of the open reading frame encoding one or more transcription factors comprise NOTCH1 or a functional derivative thereof, and wherein the nucleic acid comprising an open reading frame encoding one or more transcription factors, the one or more transcription factors, or the activator of transcription of the open reading frame encoding one or more transcription factors induces differentiation of the PSC into an immune cell in 28 days or less. In some embodiments, the immune cell expresses FOXP3. In some embodiments, the immune cell expresses CD34. In some embodiments, the immune cell expresses CD45. In some embodiments, the immune cell expresses CD4. In some embodiments, the immune cell is a regulatory T cell. In some embodiments, the immune cell is a hematopoietic stem/progenitor cell. In some embodiments, the immune cell is a hematopoietic progenitor. In some embodiments, the immune cell is a leukocyte. In some embodiments, the nucleic acid comprising an open reading frame encoding one or more transcription factors, the one or more transcription factors, or the activator of transcription of the open reading frame encoding one or more transcription factors induces differentiation of the PSC into an immune cell in 11 days or less. In some embodiments, the nucleic acid comprising an open reading frame encoding one or more transcription factors, the one or more transcription factors, or the activator of transcription of the open reading frame encoding one or more transcription factors induces differentiation of the PSC into an immune cell in 5 days or less. In some embodiments, the nucleic acid comprising an open reading frame encoding one or more transcription factors, the one or more transcription factors, or the activator of transcription of the open reading frame encoding one or more transcription factors induces differentiation of the PSC into an immune cell in 4 days or less. In some embodiments, the nucleic acid comprising an open reading frame encoding one or more transcription factors, the one or more transcription factors, or the activator of transcription of the open reading frame encoding one or more transcription factors induces differentiation of the PSC into an immune cell in 3 days or less. In some embodiments, the nucleic acid comprising an open reading frame encoding one or more transcription factors, the one or more transcription factors, or the activator of transcription of the open reading frame encoding one or more transcription factors induces differentiation of the PSC into an immune cell in 2 days or less. In some embodiments, the nucleic acid comprising an open reading frame encoding one or more transcription factors, the one or more transcription factors, or the activator of transcription of the open reading frame encoding one or more transcription factors induces differentiation of the PSC into an immune cell in 1 day or less. In some embodiments, the one or more transcription factors further comprise a transcription factor selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors further comprise a transcription factor selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors further comprise a transcription factor selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors further comprise a transcription factor selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors comprise any one of the transcription factor combinations in Tables 1, 2, 3, 5, 6, 7, 8, 9, 10, or 11. In some embodiments, the nucleic acid comprises two or more open reading frames encoding one or more transcription factors. In some embodiments, the one or more transcription factors further comprise a transcription factor selected from the group of transcription factor families consisting of Zinc fingers C2H2-type, BAF complex, Runt-related transcription factors, BTB domain containing, basic helix-loop-helix (bHLH), Nuclear factor-KB, and basic helix-loop-helix. In some embodiments, the NOTCH1 or the functional derivative thereof comprises an intracellular domain (“ICD”) of NOTCH1. In some embodiments, the NOTCH1 or the functional derivative thereof consists of an intracellular domain (“ICD”) of NOTCH1. A population of cells comprising two or more of the PSC of any one of the instant embodiments. In some embodiments, the population of cells are adherent cells. In some embodiments, the population of cells are suspension cells. In some embodiments, at least 5% of the population of cells express FOXP3. In some embodiments, at least 10% of the population of cells express FOXP3. In some embodiments, at least 20% of the population of cells express FOXP3. In some embodiments, at least 30% of the population of cells express FOXP3. In some embodiments, at least 40% of the population of cells express FOXP3. In some embodiments, at least 1% of the population of cells express CD45. In some embodiments, at least 2% of the population of cells express CD45. In some embodiments, at least 3% of the population of cells express CD45. In some embodiments, at least 4% of the population of cells express CD45. In some embodiments, at least 5% of the population of cells express CD45. In some embodiments, at least 6% of the population of cells express CD45. In some embodiments, at least 10% of the population of cells express CD45. In some embodiments, at least 15% of the population of cells express CD45. In some embodiments, at least 20% of the population of cells express CD45. In some embodiments, at least 30% of the population of cells express CD45. In some embodiments, at least 40% of the population of cells express CD45. In some embodiments, at least 50% of the population of cells express CD45. In some embodiments, at least 1% of the population of cells express CD8. In some embodiments, at least 2% of the population of cells express CD8. In some embodiments, at least 2.5% of the population of cells express CD8. In some embodiments, at least 3% of the population of cells express CD8. In some embodiments, at least 1% of the population of cells express CD34. In some embodiments, at least 2% of the population of cells express CD34. In some embodiments, at least 2.5% of the population of cells express CD34. In some embodiments, at least 2% of the population of cells express CD4. In some embodiments, at least 3% of the population of cells express CD4. In some embodiments, at least 4% of the population of cells express CD4. In some embodiments, at least 5% of the population of cells express CD4. In some embodiments, at least 10% of the population of cells express CD4. In some embodiments, at least 15% of the population of cells express CD4. In some embodiments, at least 1% of the population of cells express CD45 and CD34. In some embodiments, at least 2% of the population of cells express CD45 and CD34. In some embodiments, at least 2.5% of the population of cells express CD45 and CD34. In some embodiments, at least 3% of the population of cells express CD45 and CD34. In some embodiments, at least 5% of the population of cells express CD45 and CD56. In some embodiments, at least 6% of the population of cells express CD45 and CD56. In some embodiments, at least 10% of the population of cells express CD45 and CD56. In some embodiments, at least 15% of the population of cells express CD45 and CD56. In some embodiments, at least 20% of the population of cells express CD45 and CD56. In some embodiments, at least 30% of the population of cells express CD45 and CD56. In some embodiments, at least 40% of the population of cells express CD45 and CD56. In some embodiments, at least 50% of the population of cells express CD45 and CD56. In some embodiments, at least 60% of the population of cells express CD45 and CD56. In some embodiments, at least 70% of the population of cells express CD45 and CD56. In some embodiments, at least 80% of the population of cells express CD45 and CD56. In some embodiments, at least 5% of the population of cells express CD45 and CD34. In some embodiments, at least 6% of the population of cells express CD45 and CD34. In some embodiments, at least 10% of the population of cells express CD45 and CD34. In some embodiments, at least 15% of the population of cells express CD45 and CD34. In some embodiments, at least 1% of the population of cells express CD14 and CD11b. In some embodiments, at least 2% of the population of cells express CD14 and CD11b. In some embodiments, at least 2.5% of the population of cells express CD14 and CD11b. In some embodiments, at least 3% of the population of cells express CD14 and CD11b.

Another aspect of the instant disclose is a method of generating a population of immune cells, the method comprising: providing one or more pluripotent stem cells (PSCs); expressing in the one or more PSCs a nucleic acid comprising an open reading frame encoding one or more transcription factors, one or more transcription factors, or an activator of transcription of the open reading frame encoding one or more transcription factors, wherein the one or more transcription factors comprise NOTCH1; and generating the population of immune cells from the one or more PSCs. In some embodiments, at least one of the immune cells expresses FOXP3. In some embodiments, at least one of the immune cells expresses CD34. In some embodiments, at least one of the immune cells expresses CD45. In some embodiments, at least one of the immune cells expresses CD4. In some embodiments, at least one of the immune cells is a regulatory T cell. In some embodiments, at least one of the immune cells is a hematopoietic stem/progenitor cell. In some embodiments, at least one of the immune cells is a hematopoietic progenitor. In some embodiments, at least one of the immune cells is a leukocyte. In some embodiments, the nucleic acid comprising an open reading frame encoding one or more transcription factors, the one or more transcription factors, or the activator of transcription of the open reading frame encoding one or more transcription factors induces differentiation of the one or more PSCs into the population of immune cells in 28 days or less. In some embodiments, the nucleic acid comprising an open reading frame encoding one or more transcription factors, the one or more transcription factors, or the activator of transcription of the open reading frame encoding one or more transcription factors induces differentiation of the one or more PSCs into the population of immune cells in 11 days or less. In some embodiments, the nucleic acid comprising an open reading frame encoding one or more transcription factors, the one or more transcription factors, or the activator of transcription of the open reading frame encoding one or more transcription factors induces differentiation of the one or more PSCs into the population of immune cells in 5 days or less. In some embodiments, the nucleic acid comprising an open reading frame encoding one or more transcription factors, the one or more transcription factors, or the activator of transcription of the open reading frame encoding one or more transcription factors induces differentiation of the one or more PSCs into the population of immune cells in 4 days or less. In some embodiments, the nucleic acid comprising an open reading frame encoding one or more transcription factors, the one or more transcription factors, or the activator of transcription of the open reading frame encoding one or more transcription factors induces differentiation of the one or more PSCs into the population of immune cells in 3 days or less. In some embodiments, the nucleic acid comprising an open reading frame encoding one or more transcription factors, the one or more transcription factors, or the activator of transcription of the open reading frame encoding one or more transcription factors induces differentiation of the one or more PSCs into the population of immune cells in 2 days or less. In some embodiments, the nucleic acid comprising an open reading frame encoding one or more transcription factors, the one or more transcription factors, or the activator of transcription of the open reading frame encoding one or more transcription factors induces differentiation of the one or more PSCs into the population of immune cells in 1 day or less. In some embodiments, the one or more transcription factors further comprise a transcription factor selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors further comprise a transcription factor selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors further comprise a transcription factor selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors further comprise a transcription factor selected from the group of transcription factor families consisting of Zinc fingers C2H2-type, BAF complex, Ring finger proteins, Forkhead boxes, GATA zinc finger domain containing, Runt-related transcription factors, BTB domain containing, basic helix-loop-helix (bHLH), Nuclear factor-KB, Zinc fingers C2H2-type Protein, phosphatase 1 regulatory subunits, Zinc fingers C2H2-type, Interferon regulatory factors, Kruppel like factors, TCF/LEF transcription factor family, Wnt enhanceosome complex, Nuclear factors of activated T-cells, IPT domain containing, MicroRNA protein coding host genes, Notch receptors, MicroRNA protein coding host genes, NF-kappa B complex subunits, RAR related orphan receptors, CUT class homeoboxes and pseudogenes, and T-box transcription factors. In some embodiments, the one or more transcription factors comprise any one of the transcription factor combinations in Tables 1, 2, 3, 5, 6, 7, 8, 9, 10, or 11. In some embodiments, the nucleic acid comprises two or more open reading frame encoding one or more transcription factors. In some embodiments, the one or more transcription factors further comprise a transcription factor selected from the group of transcription factor families consisting of Zinc fingers C2H2-type, BAF complex, Runt-related transcription factors, BTB domain containing, basic helix-loop-helix (bHLH), Nuclear factor-KB, and basic helix-loop-helix. In some embodiments, the NOTCH1 or the functional derivative thereof comprises an intracellular domain (“ICD”) of NOTCH1. In some embodiments, the NOTCH1 or the functional derivative thereof consists of an intracellular domain (“ICD”) of NOTCH1. In some embodiments, the population of immune cells are adherent cells. In some embodiments, the population of immune cells are suspension cells. In some embodiments, at least 5% of the population of immune cells express FOXP3. In some embodiments, at least 10% of the population of immune cells express FOXP3. In some embodiments, at least 20% of the population of immune cells express FOXP3. In some embodiments, at least 30% of the population of immune cells express FOXP3. In some embodiments, at least 40% of the population of immune cells express FOXP3. In some embodiments, at least 1% of the population of immune cells express CD45. In some embodiments, at least 2% of the population of immune cells express CD45. In some embodiments, at least 3% of the population of immune cells express CD45. In some embodiments, at least 4% of the population of immune cells express CD45. In some embodiments, at least 5% of the population of immune cells express CD45. In some embodiments, at least 6% of the population of immune cells express CD45. In some embodiments, at least 10% of the population of immune cells express CD45. In some embodiments, at least 15% of the population of immune cells express CD45. In some embodiments, at least 20% of the population of immune cells express CD45. In some embodiments, at least 30% of the population of immune cells express CD45. In some embodiments, at least 40% of the population of immune cells express CD45. In some embodiments, at least 50% of the population of immune cells express CD45. In some embodiments, at least 1% of the population of immune cells express CD8. In some embodiments, at least 2% of the population of immune cells express CD8. In some embodiments, at least 2.5% of the population of immune cells express CD8. In some embodiments, at least 3% of the population of immune cells express CD8. In some embodiments, at least 1% of the population of immune cells express CD34. In some embodiments, at least 2% of the population of immune cells express CD34. In some embodiments, at least 2.5% of the population of immune cells express CD34. In some embodiments, at least 2% of the population of immune cells express CD4. In some embodiments, at least 3% of the population of immune cells express CD4. In some embodiments, at least 4% of the population of immune cells express CD4. In some embodiments, at least 5% of the population of immune cells express CD4. In some embodiments, at least 10% of the population of immune cells express CD4. In some embodiments, at least 15% of the population of immune cells express CD4. In some embodiments, at least 1% of the population of immune cells express CD45 and CD34. In some embodiments, at least 2% of the population of immune cells express CD45 and CD34. In some embodiments, at least 2.5% of the population of immune cells express CD45 and CD34. In some embodiments, at least 3% of the population of immune cells express CD45 and CD34. In some embodiments, at least 5% of the population of immune cells express CD45 and CD56. In some embodiments, at least 6% of the population of immune cells express CD45 and CD56. In some embodiments, at least 10% of the population of immune cells express CD45 and CD56. In some embodiments, at least 15% of the population of immune cells express CD45 and CD56. In some embodiments, at least 20% of the population of immune cells express CD45 and CD56. In some embodiments, at least 30% of the population of immune cells express CD45 and CD56. In some embodiments, at least 40% of the population of immune cells express CD45 and CD56. In some embodiments, at least 50% of the population of immune cells express CD45 and CD56. In some embodiments, at least 60% of the population of immune cells express CD45 and CD56. In some embodiments, at least 70% of the population of immune cells express CD45 and CD56. In some embodiments, at least 80% of the population of immune cells express CD45 and CD56. In some embodiments, at least 5% of the population of immune cells express CD45 and CD34. In some embodiments, at least 6% of the population of immune cells express CD45 and CD34. In some embodiments, at least 10% of the population of immune cells express CD45 and CD34. In some embodiments, at least 15% of the population of immune cells express CD45 and CD34. In some embodiments, at least 1% of the population of cells express CD14 and CD11b. In some embodiments, at least 2% of the population of cells express CD14 and CD11b. In some embodiments, at least 2.5% of the population of cells express CD14 and CD11b. In some embodiments, at least 3% of the population of cells express CD14 and CD11b.

Another aspect of the present disclosure is an isolated population of immune cells, wherein at least 5%, 6%, 10%, 15%, 20%, 30%, 40%, 50%, 60%, 70%, or 80% of the population of immune cells is engineered to express CD45 and CD56. In some embodiments, at least 1%, 2%, 2.5%, or 3% of the population of immune cells is engineered to express CD14 and CD11b. In some embodiments, at least 1%, 2%, 2.5%, or 3% of the population of immune cells is engineered to express CD45 and CD34.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts FOXP3-induced expression in treated 5C3 stem cells in ImmunoCult media and mTeSR1 stem cell media. Adherent and suspension cells are depicted.

FIG. 2 depicts exemplary markers of immune cell development.

FIG. 3 depicts the CD45 expression of cells to which various transcription factor recipes were delivered.

FIG. 4 depicts the CD45 expression of cells with transcription factor (TF) induction and no TF induction.

FIG. 5 depicts the CD4 expression of cells to which various transcription factor recipes were delivered.

FIG. 6 depicts the CD34 expression of cells with transcription factor (TF) induction and no TF induction.

FIGS. 7A-7D illustrates the CD45 expression in cells with or without transcription factor (TF) induction. FIGS. 7A-7C show representative flow cytometry and corresponding gating for TRA-1-60 (iPSC marker) and CD45 (hematopoietic lineage marker). FIG. 7D shows quantification of percentage of cells with CD45 expression in uninduced cells (D2-Uninduced) or induced cells transfected with a unique combination of TFs.

FIGS. 8A-8D illustrates the CD4 expression in cells with or without transcription factor (TF) induction. FIGS. 8A-8C show representative flow cytometry and corresponding gating for TRA-1-60 (iPSC marker) and CD4 (T helper marker marker). FIG. 8D shows quantification of percentage of cells with CD4 expression in uninduced cells (D2-Uninduced) or induced cells transfected with a unique combination of TFs.

FIGS. 9A-9D illustrates the CD8 expression in cells with or without transcription factor (TF) induction. FIGS. 9A-9C shows representative flow cytometry and corresponding gating for marker TRA-1-60 (iPSC marker) and CD8 (cytotoxic T cell marker). FIG. 9D shows quantification of percentage of cells with CD8 expression in uninduced cells (D2-Uninduced) or induced cells transfected with a unique combination of TFs.

FIGS. 10A-10D illustrates the CD45 expression in cells with or without transcription factor (TF) induction. FIGS. 10A-10C show representative flow cytometry and corresponding gating for marker TRA-1-60 (iPSC marker) and CD45 (hematopoietic lineage marker). FIG. 10D shows quantification of percentage of cells with CD45 expression in uninduced cells (D3-Uninduced) or induced cells transfected with combinations of TFs.

FIGS. 11A-11D illustrates the CD8 expression in cells with or without transcription factor (TF) induction. FIGS. 11A-11C show representative flow cytometry and corresponding gating for marker TRA-1-60 (iPSC marker) and CD8 (cytotoxic T cell marker). FIG. 11D shows quantification of percentage of cells with CD8 expression in uninduced cells (D3-Uninduced) or induced cells transfected with a unique combination of TFs.

FIGS. 12A-12L illustrates the expression of single CD45 marker or expression of double positive markers CD45+ CD56+, CD34+ CD45+ and CD14/CD11b+ in cells with or without transcription factor (TF) induction. FIGS. 12A-12H show representative flow cytometry and corresponding gating for TF uninduced (FIGS. 12A-12D) and induced cells (FIGS. 12E-12H): TRA-1-60 (iPSC marker), CD45 (hematopoietic lineage marker), CD34 (hematopoietic stem/progenitor cell marker), CD56 (NK cell marker), CD14/CD11b (myeloid marker). FIGS. 12I-12L shows quantification of percentage of cells CD45 (FIG. 12I), CD34+CD45+ double positive (FIG. 12J), CD45+CD56+ double positive (FIG. 12K), and CD14+/CD11b+ double positive (FIG. 12L).

FIGS. 13A-13C illustrates the CD45 expression in cells with or without transcription factor (TF) induction. FIGS. 13A-13B show representative flow plots and gating strategy. FIG. 13C shows quantification of CD45 expression in uninduced and induced cells.

FIGS. 14A-14L illustrates that TF recipe D2-21's top performers induce expression of single CD45 marker or expression of double positive markers CD45+ CD56+, CD34+ CD45+ and CD14/CD11b+ in cells with transcription factor (TF) induction. Bar plots (FIGS. 14A, 14D, 14G, 14J) show representative flow cytometry for percentage of cells expressing the indicated marker(s). Corresponding gating for the indicated markers are shown (FIGS. 14B-14C; 14E-14F; 14H-14I; 14K-14L).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### INTRODUCTION

Pluripotent stem cells (PSCs) are characterized by their ability to self-renew, while maintaining potency for therapeutic applications. PSCs also have the ability to differentiate into a vast number of distinct phenotypes (e.g., immune cells). Disclosed herein are various compositions, formulations, and methods that facilitate efficient differentiation of PSCs to immune cells. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, the immune cell expresses CD34. In some embodiments, the immune cell expresses CD4. In some embodiments, the immune cell expresses CD8.

The immune cells generated from the utilization of the compositions, formulations, and methods disclosed herein can be used for therapeutic applications wherein classical lineage immune cells have been conventionally used. Thus, the various compositions, formulations, and methods disclosed herein provide robust improvements to therapeutics as the compositions, formulations, and methods disclosed herein enable efficient and repeatable PSC differentiation into potent immune cells.

### Pluripotent Stem Cells

Pluripotent stem cells (PSCs) may be characterized by self-renewal and potency. PSCs are capable of dividing indefinitely and producing identical daughter cells. When provided with a signal, PSCs may differentiate into various phenotypes. In an example, a PSC is an embryonic stem cell (ESC). In another example, a PSC is an induced PSC (iPSC). PSCs (e.g., ESCs and iPSCs) may express TRA-1-60. TRA-1-60 expression may be indicative of the cells' ability to differentiate.

Induced pluripotent stem cells (iPSCs) are a type of pluripotent stem cell derived from adult somatic cells that have been genetically reprogrammed to an embryonic stem (ES) cell-like state through the expression of genes and factors important for maintaining the defining properties of ES cells. iPSCs are like ES cells in many aspects, including the expression of ES cell markers, chromatin methylation patterns, embryoid body formation, teratoma formation, viable chimera formation, pluripotency, and the ability to contribute to many different tissues in vitro. Studies have reported a directed differentiation of iPSCs into a variety of functional cell types in vitro, and cell therapy effects of implanted iPSCs have been demonstrated in several animal models of disease. Directed differentiation is a bioengineering method that harnesses the potential of stem cells by constraining their differentiation in vitro toward a specific cell type or tissue of interest. Directed differentiation may be primarily applied to PSCs of mammalian origin, for example, mouse and human cells for biomedical research applications. Cell differentiation may involve a transformation from a proliferative mode toward differentiation mode. Directed differentiation may comprise mimicking developmental cultures in controlled conditions involving specific substrate or extracellular matrices promoting cell adhesion and differentiation and define culture media compositions. Signaling factors, such as growth factors or small molecules may be applied sequentially or in a combinatorial manner, at varying dosage and exposure time, to modulate differentiate. Direct reprogramming, also known as trans-differentiation or direct conversion, may comprise overexpressing one or several factors, introduced in the cells. In an example, the one or several factors may be transcription factors. Proper differentiation of the cell type of interest may be verified by analyzing cell type specific markers, gene expression profile, and functional assays.

The present disclosure provides pluripotent stem cells (PSCs). The PSC may comprise a nucleic acid comprising an open reading frame encoding one or more transcription factors. Transcription factors as disclosed herein may be exogenous transcription factors. The nucleic acid may comprise two or more open reading frames encoding one or more transcription factors. The PSC may comprise one or more transcription factors. The PSC may comprise an activator of transcription of the open reading frame encoding one or more transcription factors. The activator may be a CRISPR activator of the one or more transcription factors. The activator may be a small molecule activator that induces expression of the one or more transcription factors. The one or more transcription factors may be exogenous. The one or more transcription factors may be endogenous. The nucleic acid comprising an open reading frame encoding one or more transcription factors, the one or more transcription factors, or the activator of transcription of the open reading frame encoding one or more transcription factors may induce differentiation of the PSC into an immune cell. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, the immune cell expresses CD34. In some embodiments, the immune cell expresses CD4. In some embodiments, the immune cell expresses CD8. The nucleic acid comprising an open reading frame encoding one or more transcription factors, the one or more transcription factors, or the activator of transcription of the open reading frame encoding one or more transcription factors may induce the differentiation of the PSC into the immune cell in about 30 days, in about 29 days, in about 28 days, in about 27 days, in about 26 days, in about 25 days, in about 24 days, in about 23 days, in about 22 days, in about 21 days, in about 20 days, in about 19 days, in about 18 days, in about 17 days, in about 16 days, in about 15 days, in about 14 days, in about 13 days, in about 12 days, in about 11 days, in about 10 days, in about 9 days, in about 8 days, in about 7 days, in about 6 days, in about 5 days, in about 4 days, in about 3 days, in about 2 days, in about 1 day, or less. The nucleic acid comprising an open reading frame encoding one or more transcription factors, the one or more transcription factors, or the activator of transcription of the open reading frame encoding one or more transcription factors may induce the differentiation of the PSC into the immune cell in 28 days or less. The nucleic acid comprising an open reading frame encoding one or more transcription factors, the one or more transcription factors, or the activator of transcription of the open reading frame encoding one or more transcription factors may induce the differentiation of the PSC into the immune cell in 11 days or less. The nucleic acid comprising an open reading frame encoding one or more transcription factors, the one or more transcription factors, or the activator of transcription of the open reading frame encoding one or more transcription factors may induce the differentiation of the PSC into the immune cell in 4 days or less. The nucleic acid comprising an open reading frame encoding one or more transcription factors, the one or more transcription factors, or the activator of transcription of the open reading frame encoding one or more transcription factors may induce the differentiation of the PSC into the immune cell in 3 days or less, or 1 day or less. The PSC may be provided in a media. The media may not have to be altered during the differentiation of the immune cell. A population of cells comprising one or more immune cells may be generated. The population of cells may comprise adherent cells. The population of cells may comprise suspension cells. The population of cells may comprise adherent cells and suspension cells. The population of cells may be provided in a media. The media may not have to be altered during the differentiation of the PSCs into immune cells. The media may not need any nutrients, growth factors, or microenvironmental or matrix optimizations.

### Immune Cells

The ability of the immune system to control the inflammatory response and prevent reactions against self-antigens or harmless environmental molecules may be acquired during fetal life or early after birth. Immune cells, such as T cells, have various characteristics and functions that assist in the development and maintenance of the immune system. T cells develop from hematopoietic stem cells (HSCs) in the bone marrow. Selection and acquisition of both effector and regulatory functions may occur in the thymus for T cells. Various markers of T cells may be correlated with various stages of T cell development. Two examples of markers of hematopoietic progenitors are the proteins CD34 and CD45. CD34 is a cell surface marker that may aid in the attachment of hematopoietic cells in the bone marrow and may identify early hematopoietic lineages. CD45 is a protein tyrosine phosphatase that may be expressed in leukocytes and may be a marker of committed progenitors. It may be expressed throughout the progenitor stage and in mature T cells. CD4 is a co-receptor for the T Cell Receptor (TCR) that may play a role in the recognition of antigen presenting cell binding to the Major Histocompatibility Complex II (MHC class II) protein complex. The CD8 antigen is a cell surface glycoprotein found on most cytotoxic T lymphocytes that mediates efficient cell-cell interactions within the immune system. CD8 serves both as an adhesion molecule for class I MHC molecules and as a coreceptor with the TCR for T cell activation.

The present disclosure provides an immune cell. The immune cell may comprise a nucleic acid comprising an open reading frame encoding one or more transcription factors. Transcription factors as disclosed herein may be exogenous transcription factors. The nucleic acid may comprise two or more open reading frames encoding one or more transcription factors. The immune cell may comprise one or more transcription factors. The immune cell may comprise an activator of transcription of the open reading frame encoding one or more transcription factors, wherein the nucleic acid. The activator may be a CRISPR activator of the one or more transcription factors. The activator may be a small molecule activator that induces expression of the one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, the immune cell expresses CD34. In some embodiments, the immune cell expresses CD4. In some embodiments, the immune cell expresses CD8. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 4 days or less, or 1 day or less.

The present disclosure provides a FOXP3-expressing cell. The FOXP3-expressing cell may comprise a nucleic acid comprising an open reading frame encoding one or more transcription factors. Transcription factors as disclosed herein may be exogenous transcription factors. The nucleic acid may comprise two or more open reading frames encoding one or more transcription factors. The FOXP3-expressing cell may comprise one or more transcription factors. The FOXP3-expressing cell may comprise an activator of transcription of the open reading frame encoding one or more transcription factors. In some embodiments, the immune cell is a T cell. In some embodiments, the FOXP3-expressing cell is a regulatory T cell. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the FOXP3-expressing cell. In some embodiments, the FOXP3-expressing cell is provided in a media. The media may not have to be altered during the differentiation of the PSC into the FOXP3-expressing cell. In some embodiments, the nucleic acid comprising the open reading frame encoding the one or more transcription factors, the one or more transcription factors, or the activator of transcription of the open reading frame encoding one or more transcription factors induces the differentiation of the FOXP3-expressing cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 4 days or less, or 1 day or less.

The present disclosure provides a CD45-expressing cell. The CD45-expressing cell may comprise a nucleic acid comprising an open reading frame encoding one or more transcription factors. Transcription factors as disclosed herein may be exogenous transcription factors. The nucleic acid may comprise two or more open reading frames encoding one or more transcription factors. The CD45-expressing cell may comprise one or more transcription factors. The CD45-expressing cell may comprise an activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the CD45-expressing cell is a progenitor T cell. In some embodiments, the CD45-expressing cell is a mature T cell. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the CD45-expressing cell. In some embodiments, the CD45-expressing cell is provided in a media. The media may not have to be altered during the differentiation of the PSC into the CD45-expressing cell. In some embodiments, the nucleic acid comprising the open reading frame encoding the one or more transcription factors, the one or more transcription factors, or the activator of transcription of the open reading frame encoding one or more transcription factors induces the differentiation of the CD45-expressing cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 4 days or less, or 1 day or less.

The present disclosure provides a CD34-expressing cell. The CD34-expressing cell may comprise a nucleic acid comprising an open reading frame encoding one or more transcription factors. Transcription factors as disclosed herein may be exogenous transcription factors. The nucleic acid may comprise two or more open reading frames encoding one or more transcription factors. The CD34-expressing cell may comprise one or more transcription factors. The CD34-expressing cell may comprise an activator of transcription of the open reading frame encoding one or more transcription factors. In some embodiments, the immune cell is a T cell. In some embodiments, the CD34-expressing cell is a progenitor T cell. In some embodiments, the CD34-expressing cell is a mature T cell. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the CD34-expressing cell. In some embodiments, the CD34-expressing cell is provided in a media. The media may not have to be altered during the differentiation of the PSC into the CD34-expressing cell. In some embodiments, the nucleic acid comprising the open reading frame encoding the one or more transcription factors, the one or more transcription factors, or the activator of transcription of the open reading frame encoding one or more transcription factors induces the differentiation of the CD34-expressing cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 4 days or less, or 1 day or less.

The present disclosure provides a CD4-expressing cell. The CD4-expressing cell may comprise a nucleic acid comprising an open reading frame encoding one or more transcription factors. Transcription factors as disclosed herein may be exogenous transcription factors. The nucleic acid may comprise two or more open reading frames encoding one or more transcription factors. The CD4-expressing cell may comprise one or more transcription factors. The CD4-expressing cell may comprise an activator of transcription of the open reading frame encoding one or more transcription factors. In some embodiments, the immune cell is a T cell. In some embodiments, the CD4-expressing cell is a progenitor T cell. In some embodiments, the CD4-expressing cell is a mature T cell. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the CD4-expressing cell. In some embodiments, the CD4-expressing cell is provided in a media. The media may not have to be altered during the differentiation of the PSC into the CD4-expressing cell. In some embodiments, the nucleic acid comprising the open reading frame encoding the one or more transcription factors, the one or more transcription factors, or the activator of transcription of the open reading frame encoding one or more transcription factors induces the differentiation of the CD4-expressing cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 4 days or less, 1 day or less.

The present disclosure provides a T cell comprising a nucleic acid comprising an open reading frame encoding one or more transcription factors, one or more transcription factors, or an activator of transcription of the open reading frame encoding one or more transcription factors. Transcription factors as disclosed herein may be exogenous transcription factors. In some embodiments, the T cell is a regulatory T cell. In some embodiments, the T cell is a progenitor T cell. In some embodiments, the T cell is a mature T cell. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the T cell. In some embodiments, the T cell is provided in a media. The media may not have to be altered during the differentiation of the PSC into the T cell. In some embodiments, the nucleic acid comprising the open reading frame encoding the one or more transcription factors, the one or more transcription factors, or the activator of transcription of the open reading frame encoding one or more transcription factors induces the differentiation of the T cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 4 days or less, 1 day or less.

The present disclosure provides a Regulatory T cell comprising a nucleic acid comprising an open reading frame encoding one or more transcription factors, one or more transcription factors, or an activator of transcription of the open reading frame encoding one or more transcription factors. Transcription factors as disclosed herein may be exogenous transcription factors. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the Regulatory T cell. In some embodiments, the Regulatory T cell is provided in a media. The media may not have to be altered during the differentiation of the PSC into the Regulatory T cell. In some embodiments, the nucleic acid comprising the open reading frame encoding the one or more transcription factors, the one or more transcription factors, or the activator of transcription of the open reading frame encoding one or more transcription factors induces the differentiation of the Regulatory T cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 4 days or less, 1 day or less.

The present disclosure provides a T cell progenitor cell comprising a nucleic acid comprising an open reading frame encoding one or more transcription factors, one or more transcription factors, or an activator of transcription of the open reading frame encoding one or more transcription factors, wherein the nucleic acid. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the T cell progenitor cell. Transcription factors as disclosed herein may be exogenous transcription factors. In some embodiments, the T cell progenitor cell is provided in a media. The media may not have to be altered during the differentiation of the PSC into the T cell progenitor cell. In some embodiments, the nucleic acid comprising the open reading frame encoding the one or more transcription factors, the one or more transcription factors, or the activator of transcription of the open reading frame encoding one or more transcription factors induces the differentiation of the T cell progenitor cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 4 days or less, 1 day or less.

The present disclosure provides a mature T cell comprising a nucleic acid comprising an open reading frame encoding one or more transcription factors, one or more transcription factors, or an activator of transcription of the open reading frame encoding one or more transcription factors, wherein the nucleic acid. Transcription factors as disclosed herein may be exogenous transcription factors. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the mature T cell. In some embodiments, the mature T cell is provided in a media. The media may not have to be altered during the differentiation of the PSC into the mature T cell. In some embodiments, the nucleic acid comprising the open reading frame encoding the one or more transcription factors, the one or more transcription factors, or the activator of transcription of the open reading frame encoding one or more transcription factors induces the differentiation of the mature T cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 4 days or less, 1 day or less.

The present disclosure provides a population of two or more immune cells as described herein. In some embodiments, at least one immune cell is a T cell. In some embodiments, the T cell is a regulatory T cell. In some embodiments, at least one immune cell is an earlier stage hematopoietic progenitor to a T cell. In some embodiments, at least one immune cell is a committed progenitor cell. In some embodiments, at least one immune cell is a mature T cell. In some embodiments, at least one immune cell is a hematopoietic stem/progenitor cell. In some embodiments, at least one immune cell is a NK cell. In some embodiments, at least one immune cell expresses FOXP3. In some embodiments, at least one immune cell expresses CD45. In some embodiments, at least one immune cell expresses CD34. In some embodiments, at least one immune cell expresses CD4.

### Regulatory T Cells (Tregs)

Throughout life, peripheral mechanisms of regulation control auto-reactive cells that escape initial checkpoints and regulate undesired immune responses toward commensal and environmental antigens. Peripheral tolerance may comprise active mechanisms in which regulatory T cells (Tregs) may play a major role. Tregs may exert their function in different tissues, at sites of inflammation and in close contact with T effector (Teff) cells.

Tregs may express the protein forkhead box P3 (FOXP3). FOXP3 may bind to specific regions of the DNA and help control activity of genes involved in immune system regulation. FOXP3-positive (FOXP3+) Tregs may proliferate in a limited manner in vitro and may have trouble producing cytokines, with the exception of low transforming growth factor-β (TGF-β) and interleukin-35 (IL-35). They may be, however, very responsive to interleukin-2 (IL-2), which may act through its receptor and through activation of STAT5. IL-2 is an important factor for Treg survival and maintenance in vivo and may be required at higher doses for in vitro expansion. Other factors may influence Tregs, such as TGF-β, thymic stromal lymphopoietin (TSLP), and costimulatory molecules such as CD28. Specific Treg populations have been linked to the regulation of different T helper (Th) subclasses. FOXP3 may cooperate with lineage-specific transcription factors in order to skew Treg abilities toward the regulation of diverse types of immune responses. Mouse models suggest that modulation of Treg cells can treat autoimmune disease and cancer and can facilitate organ transplantation and wound healing.

Expression of FOXP3 may be required for Treg development and may control a genetic program specifying cell fate. FOXP3 may be a potent repressor of IL-2 production and may upregulate the expression of the CD25 receptor and of the Treg marker CTLA4. FOXP3 may supports the maintenance of an immunosuppressive environment. Absence of FOXP3 in specific KO mice or in the natural mouse mutant, the scurfy mouse, may be responsible for massive lymphoproliferation and for a severe autoimmune syndrome. Mutations of FOXP3 may lead to a similar phenotype in human men. However, the establishment of a mouse strain carrying a defective FoxP3 allele has demonstrated that this gene may be essential for the function but not for the development of Tregs. Rather, FOXP3 may potentiate pre-established Treg features, such as responsiveness to IL-2 In humans, Treg differentiation may be characterized by specific demethylation of over a hundred of loci which become accessible for FOXP3 binding once it is expressed. Importantly, the CpG methylation status of DNA may regulate FoxP3 expression and accesses to its targets. The regulation of FOXP3 expression may occur at both the transcriptional and translational levels. The present disclosure provides one or more Tregs or FOXP3-expressing cells comprising one or more exogenous expression cassettes. The one or more expression cassettes may comprise one or more transcription factors. The one or more transcription factors may induce differentiation of one or more PSCs. The exogenous expression cassettes may induce the differentiation of the one or more PSCs into Tregs or FOXP3-expressing cells. The exogenous expression cassettes may induce the differentiation of the one or more PSCs into Tregs or FOXP3-expressing cells in 28 days or less. The one or more PSCs may be provided in a media. The media may not have to be altered during the differentiation of the PSC into the Treg or the FOXP3-expressing cell. The media may not need any nutrients, growth factors, or microenvironmental or matrix optimizations.

The present disclosure provides a Regulatory T cell comprising a nucleic acid comprising an open reading frame encoding one or more transcription factors, one or more transcription factors, or an activator of transcription of the open reading frame encoding one or more transcription factors. Transcription factors as disclosed herein may be exogenous transcription factors. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the Regulatory T cell. In some embodiments, the Regulatory T cell is provided in a media. The media may not have to be altered during the differentiation of the PSC into the Regulatory T cell. In some embodiments, the nucleic acid comprising the open reading frame encoding the one or more transcription factors, the one or more transcription factors, or the activator of transcription of the open reading frame encoding one or more transcription factors induces the differentiation of the Regulatory T cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides a population of cells comprising one or more regulatory T cells or FOXP3-expressing cells differentiated from one or more PSCs. The population of cells may comprise adherent cells. The population of cells may comprise suspension cells. The population of cells may comprise adherent cells and suspension cells. The population of cells may be provided in a media. The media may not have to be altered during the differentiation of the PSCs into Tregs or FOXP3-expressing cells. The media may not need any nutrients, growth factors, or microenvironmental or matrix optimizations. At least 5% of the cells may express FOXP3. At least about 5%, at least about 6%, at least about 7%, at least about 8%, at least about 9%, at least about 10%, at least about 11%, at least about 12%, at least about 13%, at least about 14%, at least about 15%, at least about 1⁶,%, at least about 17%, at least about 18%, at least about 19%, at least about 20%, at least about 25%, at least about 30%, at least about 35%, or at least about 40% of the cells may express FOXP3.

### CD45-Expressing Cells

CD45 is a molecule that may be found on the surface of nucleated hematopoietic cells and their precursors. Specifically, it is a protein tyrosine phosphatase that may be expressed in leukocytes. It may be a type I transmembrane protein. It may be a marker of committed progenitors and may be expressed throughout the progenitor stage and in mature T cells.

The present disclosure provides a CD45-expressing cell or a cell that expresses any other immune cell, or immune-associated markers disclosed herein, that are associated with the (TF family or TF, or TF combination) non-natural, exogenous induction of iPSC differentiation along the immune cell differentiation trajectory comprising a nucleic acid comprising an open reading frame encoding one or more transcription factors, one or more transcription factors, or an activator of transcription of the open reading frame encoding one or more transcription factors. Transcription factors as disclosed herein may be exogenous transcription factors. In some embodiments, the immune cell is a T cell. In some embodiments, the immune cell is a hematopoietic stem/progenitor cell. In some embodiments, the immune cell is a NK cell. In some embodiments, the CD45-expressing cell is a progenitor T cell. In some embodiments, the CD45-expressing cell is a mature T cell. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the CD45-expressing cell. In some embodiments, the CD45-expressing cell is provided in a media. The media may not have to be altered during the differentiation of the PSC into the CD45-expressing cell. In some embodiments, the nucleic acid comprising the open reading frame encoding the one or more transcription factors, the one or more transcription factors, or the activator of transcription of the open reading frame encoding one or more transcription factors induces the differentiation of the CD45-expressing cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides a population of cells comprising two or more CD45-expressing cells or two or more cells that expressing any other immune cell, or immune-associated markers disclosed herein, that are associated with the (TF family or TF, or TF combination) non-natural, exogenous induction of iPSC differentiation along the immune-cell differentiation trajectory as disclosed herein. The population of cells may comprise adherent cells. The population of cells may comprise suspension cells. The population of cells may comprise adherent cells and suspension cells. The population of cells may be provided in a media. The media may not have to be altered during the differentiation of the PSCs into immune cells or immune-like cells, or cells that express CD45, cells that express CD34 or cells that express CD4 or cells that express CD8, or cells that express immune-cell lineage markers or immune cell-associated markers disclosed herein that are associated with the (TF recipe) non-natural, exogenous induction of iPSC differentiation along the immune-cell differentiation trajectory. The population of cells may be provided in a media. The media may not need any nutrients, growth factors, or microenvironmental or matrix optimizations. At least one cell in the population of cells may express CD45. At least about 0.5% to at least about 20% of a population of cells may express CD45. At least about 0.5%, at least about 1%, at least about 2%, at least about 3%, at least about 4%, at least about 5%, at least about 6%, at least about 7%, at least about 8%, at least about 9%, at least about 10%, at least about 110, at least about 12%, at least about 13%, at least about 14%, at least about 15%, at least about 16%, at least about 17%, at least about 18%, at least about 19%, at least about 20%, or more of a population of cells may express CD45. In some embodiments, at least about 1% to at least about 6% of a population of cells may express CD45. In some embodiments, at least about 3% to at least about 9% of a population of cells may express CD45. In some embodiments, greater than 9%, 10%, 20%, 30%, 40%, 50%, 60%, 70%, 80% of a population of cells may express CD45.

In some embodiments, at least 0.01% of the immune cell express CD45 or may express any immune-cell lineage markers or immune cell-associated markers disclosed herein that are associated with the (TF recipe) non-natural, exogenous induction of iPSC differentiation along the immune-cell differentiation trajectory. In some embodiments, at least 0.1% of the immune cell express CD45. In some embodiments, at least 0.5% of the immune cell express CD45. In some embodiments, at least 1% of immune cell express CD45. In some embodiments, at least 1.5% of the immune cell express CD45. In some embodiments, at least 2% of the immune cell express CD45. In some embodiments, at least 3% of the immune cell express CD45. In some embodiments, at least 4% of the immune cell express CD45. In some embodiments, at least 5% of the immune cell express CD45. In some embodiments, at least 6% of the immune cell express CD45. In some embodiments, at least 7% of the immune cell express CD45. In some embodiments, at least 8% of the immune cell express CD45. In some embodiments, at least 9% of the immune cell express CD45. In some embodiments, at least 10% of the immune cell express CD45. In some embodiments, at least 12% of immune cell express CD45. In some embodiments, at least 15% of immune cell express CD45. In some embodiments, at least 17% of immune cell express CD45. In some embodiments, at least 20% of immune cell express CD45. In some embodiments, at least 25% of the immune cell express CD45. In some embodiments, at least 30% of the immune cell express CD45. In some embodiments, at least 35% of the immune cell express CD45. In some embodiments, at least 40% of the immune cell express CD45. In some embodiments, at least 45% of the immune cell express CD45. In some embodiments, at least 50% of the immune cell express CD45. In some embodiments, at least 60% of the immune cell express CD45. In some embodiments, at least 70% of the immune cell express CD45. In some embodiments, at least 80% of the immune cell express CD45. In some embodiments, at least 90% of the immune cell express CD45. In some embodiments, at least 95% of the immune cell express CD45. In some embodiments, at least 99% of the immune cell express CD45.

In some embodiments, the percentage of the immune cell express CD45, or a cell that expresses any other immune-cell markers or markers associated with immune cell lineage disclosed herein, that are associated with the (a TF family, TF, TF combination disclosed) non-natural, exogenous induction of iPSC differentiation along the immune-cell differentiation trajectory is at least about 1% to about 50% or at least about 1% to greater than 50%, 60%, 70%, or 80%. In some embodiments, the portion of the population of cells that expresses CD45 is at least about 1% to about 6.5%. In some embodiments, the portion of the population of cells that expresses CD45 is at least about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1% to about 5.5%, about 1% to about 6%, about 1% to about 6.5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 1.5% to about 5.5%, about 1.5% to about 6%, about 1.5% to about 6.5%, about 2% to about 2.5%, about 2% to about 3%, about 2% to about 3.5%, about 2% to about 4%, about 2% to about 4.5%, about 2% to about 5%, about 2% to about 5.5% about 2% to about 6%, about 2% to about 6.5%, about 2.5% to about 3%, about 2.5% to about 3.5%, about 2.5% to about 4%, about 2.5% to about 4.5%, about 2.5% to about 5%, about 2.5% to about 5.5%, about 2.5% to about 6%, about 2.5% to about 6.5%, about 3% to about 3.5%, about 3% to about 4%, about 3% to about 4.5%, about 3% to about 5%, about 3% to about 5.5%, about 3% to about 6%, about 3% to about 6.5%, about 3.5% to about 4%, about 3.5% to about 4.5%, about 3.5% to about 5%, about 3.5% to about 5.5%, about 3.5% to about 6%, about 3.5% to about 6.5%, about 4% to about 4.5%, about 4% to about 5%, about 4% to about 5.5%, about 4% to about 6%, about 4% to about 6.5%, about 4.5% to about 5%, about 4.5% to about 5.5%, about 4.5% to about 6%, about 4.5% to about 6.5%, about 5% to about 5.5%, about 5% to about 6%, about 5% to about 6.5%, about 5.5% to about 6%, about 5.5% to about 6.5%, or about 6% to about 6.5%. In some embodiments, the portion of the population of cells that expresses CD45 is at least about 1%, about 1.5%, about 2%, about 2.5%, about 3%, about 3.5%, about 4%, about 4.5%, about 5%, about 5.5%, about 6%, or about 6.5%. In some embodiments, the portion of the population of cells that expresses CD45 is at least at least about 1%, about 1.5%, about 2%, about 2.5%, about 3%, about 3.5%, about 4%, about 4.5%, about 5%, about 5.5%, or about 6%. In some embodiments, the portion of the population of cells that expresses CD45 is at least at most about 1.5%, about 2%, about 2.5%, about 3%, about 3.5%, about 4%, about 4.5%, about 5%, about 5.5%, about 6%, about 6.5%, In some embodiments, the portion of the population of cells that expresses CD45 is at least about 7% to about 50%. In some embodiments, the portion of the population of cells that expresses CD45 is at least about 7% to about 7.5%, about 7% to about 8%, about 7% to about 8.5%, about 7% to about 9%, about 7% to about 9.5%, about 7% to about 10%, about 7% to about 15%, about 7% to about 20%, about 7% to about 30%, about 7% to about 40%, about 7% to about 50%, about 7.5% to about 8%, about 7.5% to about 8.5%, about 7.5% to about 9%, about 7.5% to about 9.5%, about 7.5% to about 10%, about 7.5% to about 15%, about 7.5% to about 20%, about 7.5% to about 30%, about 7.5% to about 40%, about 7.5% to about 50%, about 8% to about 8.5%, about 8% to about 9%, about 8% to about 9.5%, about 8% to about 10%, about 8% to about 15%, about 8% to about 20%, about 8% to about 30%, about 8% to about 40%, about 8% to about 50%, about 8.5% to about 9%, about 8.5% to about 9.5%, about 8.5% to about 10%, about 8.5% to about 15%, about 8.5% to about 20%, about 8.5% to about 30%, about 8.5% to about 40%, about 8.5% to about 50%, about 9% to about 9.5%, about 9% to about 10%, about 9% to about 15%, about 9% to about 20%, about 9% to about 30%, about 9% to about 40%, about 9% to about 50%, about 9.5% to about 10%, about 9.5% to about 15%, about 9.5% to about 20%, about 9.5% to about 30%, about 9.5% to about 40%, about 9.5% to about 50%, about 10% to about 15%, about 10% to about 20%, about 10% to about 30%, about 10% to about 40%, about 10% to about 50%, about 15% to about 20%, about 15% to about 30%, about 15% to about 40%, about 15% to about 50%, about 20% to about 30%, about 20% to about 40%, about 20% to about 50%, about 30% to about 40%, about 30% to about 50%, or about 40% to about 50%. In some embodiments, the portion of the population of cells that expresses CD45 is at least about 7%, about 7.5%, about 8%, about 8.5%, about 9%, about 9.5%, about 10%, about 15%, about 20%, about 30%, about 40%, or about 50%. In some embodiments, the portion of the population of cells that expresses CD45 is at least at least about 7%, about 7.5%, about 8%, about 8.5%, about 9%, about 9.5%, about 10%, about 15%, about 20%, about 30%, or about 40%. In some embodiments, the portion of the population of cells that expresses CD45 is at least at most about 7.5%, about 8%, about 8.5%, about 9%, about 9.5%, about 10%, about 15%, about 20%, about 30%, about 40%, or about 50%. In some embodiments, the portion of the population of cells that expresses CD45 is greater than 50%.

### CD34-Expressing Cells

The present disclosure provides a CD34-expressing cell or a cell that expresses any other immune-cell markers or markers associated with immune cell lineage disclosed herein comprising a nucleic acid comprising an open reading frame encoding one or more transcription factors, one or more transcription factors, or an activator of transcription of the open reading frame encoding one or more transcription factors. Transcription factors as disclosed herein may be non-natural or exogenous transcription factors. In some embodiments, the immune cell is a T cell. In some embodiments, the immune cell is any immune-cell expressing the immune cell markers or markers associated with immune cell lineage disclosed herein. In some embodiments, the CD34-expressing cell is a progenitor T cell. In some embodiments, the CD34-expressing cell is a mature T cell. In some embodiments, the CD34-expressing cell is a hematopoietic stem/progenitor cell. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the CD34-expressing cell. In some embodiments, the CD34-expressing cell is provided in a media. The media may not have to be altered during the differentiation of the PSC into the CD34-expressing cell. In some embodiments, the nucleic acid comprising the open reading frame encoding the one or more transcription factors, the one or more transcription factors, or the activator of transcription of the open reading frame encoding one or more transcription factors induces the differentiation of the CD34-expressing cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides a population of cells comprising two or more CD34-expressing cells as disclosed herein. At least one cell in the population of cells may express CD34. At least about 0.5% to at least about 20% of a population of cells may express CD34. At least about 0.5%, 1%, 2%, 3%, 4%, 5%, 6%, 7%, 8%, 9%, 10%, 11%, 12%, 13%, 14%, 15%, 16%, 17%, 18%, 19%, 20%, or more of a population of cells may express CD34. In some embodiments, at least about 2% to at least about 2.5% of a population of cells may express CD34. In some embodiments, the portion of the population of cells that expresses CD34 is at least about 1% to about 6.5%. In some embodiments, the portion of the population of cells that expresses CD34 may be greater than 6.5%. In some embodiments, the portion of the population of cells that expresses CD34 is at least about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1% to about 5.5%, about 1% to about 6%, about 1% to about 6.5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 1.5% to about 5.5%, about 1.5% to about 6%, about 1.5% to about 6.5%, about 2% to about 2.5%, about 2% to about 3%, about 2% to about 3.5%, about 2% to about 4%, about 2% to about 4.5%, about 2% to about 5%, about 2% to about 5.5%, about 2% to about 6%, about 2% to about 6.5%, about 2.5% to about 3%, about 2.5% to about 3.5%, about 2.5% to about 4%, about 2.5% to about 4.5%, about 2.5% to about 5%, about 2.5% to about 5.5%, about 2.5% to about 6%, about 2.5% to about 6.5%, about 3% to about 3.5%, about 3% to about 4%, about 3% to about 4.5%, about 3% to about 5%, about 3% to about 5.5%, about 3% to about 6%, about 3% to about 6.5%, about 3.5% to about 4%, about 3.5% to about 4.5%, about 3.5% to about 5%, about 3.5% to about 5.5%, about 3.5% to about 6%, about 3.5% to about 6.5%, about 4% to about 4.5%, about 4% to about 5%, about 4% to about 5.5%, about 4% to about 6%, about 4% to about 6.5%, about 4.5% to about 5%, about 4.5% to about 5.5%, about 4.5% to about 6%, about 4.5% to about 6.5%, about 5% to about 5.5%, about 5% to about 6%, about 5% to about 6.5%, about 5.5% to about 6%, about 5.5% to about 6.5%, or about 6% to about 6.5%. In some embodiments, the portion of the population of cells that expresses CD34 is at least about 1%, about 1.5%, about 2%, about 2.5%, about 3%, about 3.5%, about 4%, about 4.5%, about 5%, about 5.5%, about 6%, or about 6.5%. In some embodiments, the portion of the population of cells that expresses CD34 is at least at least about 1%, about 1.5%, about 2%, about 2.5%, about 3%, about 3.5%, about 4%, about 4.5%, about 5%, about 5.5%, or about 6%. In some embodiments, the portion of the population of cells that expresses CD34 is at least at most about 1.5%, about 2%, about 2.5%, about 3%, about 3.5%, about 4%, about 4.5%, about 5%, about 5.5%, about 6%, or about 6.5%.

### CD4-Expressing Cells

The present disclosure provides a CD4-expressing cell or a cell that expresses any other immune-cell markers or markers associated with immune cell lineage disclosed herein comprising a nucleic acid comprising an open reading frame encoding one or more transcription factors, one or more transcription factors, or an activator of transcription of the open reading frame encoding one or more transcription factors. Transcription factors as disclosed herein may be exogenous transcription factors. In some embodiments, the immune cell is a T cell. In some embodiments, the immune cell is any immune-cell expressing the immune cell markers or markers associated with immune cell lineage disclosed herein. In some embodiments, the CD4-expressing cell is a progenitor T cell. In some embodiments, the CD4-expressing cell is a mature T cell. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the CD4-expressing cell. In some embodiments, the CD4-expressing cell is provided in a media. The media may not have to be altered during the differentiation of the PSC into the CD4-expressing cell. In some embodiments, the nucleic acid comprising the open reading frame encoding the one or more transcription factors, the one or more transcription factors, or the activator of transcription of the open reading frame encoding one or more transcription factors induces the differentiation of the CD4-expressing cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides a population of cells comprising two or more CD4-expressing cells as disclosed herein. At least one cell in the population of cells may express CD4. At least about 0.5% to at least about 20% of a population of cells may express CD4. At least about 0.5%, 1%, 2%, 3%, 4%, 5%, 6%, 7%, 8%, 9%, 10%, 11%, 12%, 13%, 14%, 15%, 16%, 17%, 18%, 19%, 20%, or more of a population of cells may express CD4. In some embodiments, at least about 2% to at least about 5% of a population of cells may express CD4. In some embodiments, the portion of the population of cells that expresses CD4 is at least about 1% to about 6.5%. In some embodiments, the portion of the population of cells that expresses CD4 is at least about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1% to about 5.5%, about 1% to about 6%, about 1% to about 6.5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 1.5% to about 5.5%, about 1.5% to about 6%, about 1.5% to about 6.5%, about 2% to about 2.5%, about 2% to about 3%, about 2% to about 3.5%, about 2% to about 4%, about 2% to about 4.5%, about 2% to about 5%, about 2% to about 5.5%, about 2% to about 6%, about 2% to about 6.5%, about 2.5% to about 3%, about 2.5% to about 3.5% about 2.5% to about 4%, about 2.5% to about 4.5%, about 2.5% to about 5%, about 2.5% to about 5.5%, about 2.5% to about 6%, about 2.5% to about 6.5%, about 3% to about 3.5%, about 3% to about 4%, about 3% to about 4.5%, about 3% to about 5%, about 3% to about 5.5%, about 3% to about 6%, about 3% to about 6.5%, about 3.5% to about 4%, about 3.5% to about 4.5%, about 3.5% to about 5%, about 3.5% to about 5.5%, about 3.5% to about 6%, about 3.5% to about 6.5%, about 4% to about 4.5%, about 4% to about 5%, about 4% to about 5.5%, about 4% to about 6%, about 4% to about 6.5%, about 4.5% to about 5%, about 4.5% to about 5.5%, about 4.5% to about 6%, about 4.5% to about 6.5%, about 5% to about 5.5%, about 5% to about 6%, about 5% to about 6.5%, about 5.5% to about 6%, about 5.5% to about 6.5%, or about 6% to about 6.5%. In some embodiments, the portion of the population of cells that expresses CD4 is at least about 1%, about 1.5%, about 2%, about 2.5%, about 3%, about 3.5%, about 4%, about 4.5%, about 5%, about 5.5%, about 6%, or about 6.5%. In some embodiments, the portion of the population of cells that expresses CD4 is at least at least about 1%, about 1.5%, about 2%, about 2.5%, about 3%, about 3.5%, about 4%, about 4.5%, about 5%, about 5.5%, or about 6%. In some embodiments, the portion of the population of cells that expresses CD4 is at least at most about 1.5%, about 2%, about 2.5%, about 3%, about 3.5%, about 4%, about 4.5%, about 5%, about 5.5%, about 6%, or about 6.5%. In some embodiments, the portion of the population of cells that expresses CD4 is at least about 7% to about 20%. In some embodiments, the portion of the population of cells that expresses CD4 is at least about 7% to about 7.5%, about 7% to about 8%, about 7% to about 8.5%, about 7% to about 9%, about 7% to about 9.5%, about 7% to about 10%, about 7% to about 12%, about 7% to about 14%, about 7% to about 15%, about 7% to about 18%, about 7% to about 20%, about 7.5% to about 8%, about 7.5% to about 8.5%, about 7.5% to about 9%, about 7.5% to about 9.5%, about 7.5% to about 10%, about 7.5% to about 12%, about 7.5% to about 14%, about 7.5% to about 15%, about 7.5% to about 18%, about 7.5% to about 20%, about 8% to about 8.5%, about 8% to about 9%, about 8% to about 9.5% about 8% to about 10%, about 8% to about 12%, about 8% to about 14%, about 8% to about 15%, about 8% to about 18%, about 8% to about 20%, about 8.5% to about 9%, about 8.5% to about 9.5%, about 8.5% to about 10%, about 8.5% to about 12%, about 8.5% to about 14%, about 8.5% to about 15%, about 8.5% to about 18%, about 8.5% to about 20%, about 9% to about 9.5%, about 9% to about 10%, about 9% to about 12%, about 9% to about 14%, about 9% to about 15%, about 9% to about 18%, about 9% to about 20%, about 9.5% to about 10%, about 9.5% to about 12%, about 9.5% to about 14%, about 9.5% to about 15%, about 9.5% to about 18%, about 9.5% to about 20%, about 10% to about 12%, about 10% to about 14%, about 10% to about 15%, about 10% to about 18%, about 10% to about 20%, about 12% to about 14%, about 12% to about 15%, about 12% to about 18%, about 12% to about 20%, about 14% to about 15%, about 14% to about 18%, about 14% to about 20%, about 15% to about 18%, about 15% to about 20%, or about 18% to about 20%. In some embodiments, the portion of the population of cells that expresses CD4 is at least about 7%, about 7.5%, about 8%, about 8.5%, about 9%, about 9.5%, about 10%, about 12%, about 14%, about 15%, about 18%, or about 20%. In some embodiments, the portion of the population of cells that expresses CD4 is at least at least about 7%, about 7.5%, about 8%, about 8.5%, about 9%, about 9.5%, about 10%, about 12%, about 14%, about 15%, or about 18%. In some embodiments, the portion of the population of cells that expresses CD4 is at least at most about 7.5%, about 8%, about 8.5%, about 9%, about 9.5%, about 10%, about 12%, about 14%, about 15%, about 18%, or about 20%. In some embodiments, the portion of the population of cells that expresses CD4 may be greater than 20%.

### CD8

The present disclosure provides a CD8-expressing cell or a cell that expresses any other immune-cell markers or markers associated with immune cell lineage disclosed herein comprising a nucleic acid comprising an open reading frame encoding one or more transcription factors, one or more transcription factors, or an activator of transcription of the open reading frame encoding one or more transcription factors. Transcription factors as disclosed herein may be exogenous transcription factors. In some embodiments, the immune cell is a T cell. In some embodiments, the immune cell is any immune-cell expressing the immune cell markers or markers associated with immune cell lineage disclosed herein. In some embodiments, the CD8-expressing cell is a progenitor T cell. In some embodiments, the CD8-expressing cell is a mature T cell. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the CD8-expressing cell. In some embodiments, the CD8-expressing cell is provided in a media. The media may not have to be altered during the differentiation of the PSC into the CD8-expressing cell. In some embodiments, the nucleic acid comprising the open reading frame encoding the one or more transcription factors, the one or more transcription factors, or the activator of transcription of the open reading frame encoding one or more transcription factors induces the differentiation of the CD8-expressing cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides a population of cells comprising two or more CD8-expressing cells as disclosed herein. At least one cell in the population of cells may express CD8. At least about 0.5% to at least about 20% of a population of cells may express CD8. At least about 0.5%, 1%, 2%, 3%, 4%, 5%, 6%, 7%, 8%, 9%, 10%, 11%, 12%, 13%, 14%, 15%, 16%, 17%, 18%, 19%, 20%, or more of a population of cells may express CD8. In some embodiments, at least about 2% to at least about 5% of a population of cells may express CD8.

In some embodiments, the portion of the population of cells that expresses CD8 is at least about 1% to about 5%. In some embodiments, the portion of the population of cells that expresses CD8 is at least about 1% to about 2%, about 1% to about 2.5%, about 1% to about 3%, about 1% to about 3.5%, about 1% to about 4%, about 1% to about 4.5%, about 1% to about 5%, about 2% to about 2.5%, about 2% to about 3%, about 2% to about 3.5%, about 2% to about 4%, about 2% to about 4.5%, about 2% to about 5%, about 2.5% to about 3%, about 2.5% to about 3.5%, about 2.5% to about 4%, about 2.5% to about 4.5%, about 2.5% to about 5%, about 3% to about 3.5%, about 3% to about 4%, about 3% to about 4.5%, about 3% to about 5%, about 3.5% to about 4%, about 3.5% to about 4.5%, about 3.5% to about 5%, about 4% to about 4.5%, about 4% to about 5%, or about 4.5% to about 5%. In some embodiments, the portion of the population of cells that expresses CD8 is at least about 1%, about 2%, about 2.5%, about 3%, about 3.5%, about 4%, about 4.5%, or about 5%. In some embodiments, the portion of the population of cells that expresses CD8 is at least at least about 1%, about 2%, about 2.5%, about 3%, about 3.5%, about 4%, or about 4.5%. In some embodiments, the portion of the population of cells that expresses CD8 is at least at most about 2%, about 2.5%, about 3%, about 3.5%, about 4%, about 4.5%, or about 5%.

### Double Positives

### CD45+ and CD34+

The present disclosure provides a CD45 and CD34-expressing cell or a cell that expresses any other immune-cell markers or markers associated with immune cell lineage disclosed herein comprising a nucleic acid comprising an open reading frame encoding one or more transcription factors, one or more transcription factors, or an activator of transcription of the open reading frame encoding one or more transcription factors. Transcription factors as disclosed herein may be exogenous transcription factors. In some embodiments, the immune cell is a hematopoietic stem/progenitor cell. In some embodiments, the immune cell is any immune-cell expressing the immune cell markers or markers associated with immune cell lineage disclosed herein. In some embodiments, the CD45 and CD34-expressing cell is an early hematopoietic stem/progenitor cell. In some embodiments, the CD45 and CD34-expressing cell is a mature hematopoietic stem/progenitor cell. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the CD45 and CD34-expressing cell. In some embodiments, the CD45 and CD34-expressing cell is provided in a media. The media may not have to be altered during the differentiation of the PSC into the CD45 and CD34-expressing cell. In some embodiments, the nucleic acid comprising the open reading frame encoding the one or more transcription factors, the one or more transcription factors, or the activator of transcription of the open reading frame encoding one or more transcription factors induces the differentiation of the CD45 and CD34-expressing cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides a population of cells comprising two or more CD45 and CD34-expressing cells as disclosed herein. At least one cell in the population of cells may express CD45 and CD34. At least about 0.5% to at least about 20% of a population of cells may express CD45 and CD34. In some embodiments, at least about 2% to at least about 5% of a population of cells may express CD45 and CD34. In some embodiments, the portion of the population of cells that expresses CD45 and CD34 is at least about 1% to about 6.5%. In some embodiments, the portion of the population of cells that expresses CD45 and CD34 is at least about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1% to about 5.5%, about 1% to about 6%, about 1% to about 6.5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 1.5% to about 5.5%, about 1.5% to about 6%, about 1.5% to about 6.5%, about 2% to about 2.5%, about 2% to about 3%, about 2% to about 3.5%, about 2% to about 4%, about 2% to about 4.5%, about 2% to about 5%, about 2% to about 5.5%, about 2% to about 6%, about 2% to about 6.5%, about 2.5% to about 3%, about 2.5% to about 3.5%, about 2.5% to about 4%, about 2.5% to about 4.5%, about 2.5% to about 5%, about 2.5% to about 5.5%, about 2.5% to about 6%, about 2.5% to about 6.5%, about 3% to about 3.5%, about 3% to about 4%, about 3% to about 4.5%, about 3% to about 5%, about 3% to about 5.5%, about 3% to about 6%, about 3% to about 6.5%, about 3.5% to about 4%, about 3.5% to about 4.5%, about 3.5% to about 5%, about 3.5% to about 5.5%, about 3.5% to about 6%, about 3.5% to about 6.5%, about 4% to about 4.5%, about 4% to about 5%, about 4% to about 5.5%, about 4% to about 6%, about 4% to about 6.5%, about 4.5% to about 5%, about 4.5% to about 5.5%, about 4.5% to about 6%, about 4.5% to about 6.5%, about 5% to about 5.5%, about 5% to about 6%, about 5% to about 6.5%, about 5.5% to about 6%, about 5.5% to about 6.5%, or about 6% to about 6.5%. In some embodiments, the portion of the population of cells that expresses CD45 and CD34 is at least about 1%, about 1.5%, about 2%, about 2.5%, about 3%, about 3.5%, about 4%, about 4.5%, about 5%, about 5.5%, about 6%, or about 6.5%. In some embodiments, the portion of the population of cells that expresses CD45 and CD34 is at least at least about 1%, about 1.5%, about 2%, about 2.5%, about 3%, about 3.5%, about 4%, about 4.5%, about 5%, about 5.5%, or about 6%. In some embodiments, the portion of the population of cells that expresses CD45 and CD34 is at least at most about 1.5%, about 2%, about 2.5%, about 3%, about 3.5%, about 4%, about 4.5%, about 5%, about 5.5%, about 6% or about 6.5%.

In some embodiments, the portion of the population of cells that expresses CD45 and CD34 is at least about 7% to about 20%. In some embodiments, the portion of the population of cells that expresses CD45 and CD34 is at least about 7% to about 7.5%, about 7% to about 8%, about 7% to about 8.5%, about 7% to about 9%, about 7% to about 10%, about 7% to about 15%, about 7% to about 20%, about 7.5% to about 8%, about 7.5% to about 8.5%, about 7.5% to about 9%, about 7.5% to about 10%, about 7.5% to about 15%, about 7.5% to about 20%, about 8% to about 8.5%, about 8% to about 9%, about 8% to about 10%, about 8% to about 15%, about 8% to about 20%, about 8.5% to about 9%, about 8.5% to about 10%, about 8.5% to about 15%, about 8.5% to about 20%, about 9% to about 10%, about 9% to about 15%, about 9% to about 20%, about 10% to about 15%, about 10% to about 20%, or about 15% to about 20%. In some embodiments, the portion of the population of cells that expresses CD45 and CD34 is at least about 7%, about 7.5%, about 8%, about 8.5%, about 9%, about 10%, about 15%, or about 20%. In some embodiments, the portion of the population of cells that expresses CD45 and CD34 is at least at least about 7%, about 7.5%, about 8%, about 8.5%, about 9%, about 10%, or about 15%. In some embodiments, the portion of the population of cells that expresses CD45 and CD34 is at least at most about 7.5%, about 8%, about 8.5%, about 9%, about 10%, about 15%, or about 20%.

### Double Positives

### CD45+ and CD56+

The present disclosure provides a CD45 and CD56-expressing cell or a cell that expresses any other immune-cell markers or markers associated with immune cell lineage disclosed herein comprising a nucleic acid comprising an open reading frame encoding one or more transcription factors, one or more transcription factors, or an activator of transcription of the open reading frame encoding one or more transcription factors. Transcription factors as disclosed herein may be exogenous transcription factors. In some embodiments, the immune cell is a NK (Natural Killer) cell. In some embodiments, the immune cell is any immune-cell expressing the immune cell markers or markers associated with immune cell lineage disclosed herein. In some embodiments, the CD45 and CD56-expressing cell is a progenitor NK cell. In some embodiments, the CD45 and CD56-expressing cell is a mature NK cell. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the CD45 and CD56-expressing cell. In some embodiments, the CD45 and CD56-expressing cell is provided in a media. The media may not have to be altered during the differentiation of the PSC into the CD45 and CD56-expressing cell. In some embodiments, the nucleic acid comprising the open reading frame encoding the one or more transcription factors, the one or more transcription factors, or the activator of transcription of the open reading frame encoding one or more transcription factors induces the differentiation of the CD45 and CD56-expressing cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides a population of cells comprising two or more CD45 and CD56-expressing cells as disclosed herein. At least one cell in the population of cells may express CD45 and CD56. At least about 0.5% to at least about 80% of a population of cells may express CD45 and CD56. In some embodiments, greater than 80% of a population of cells may express CD45 and CD56. In some embodiments, the portion of the population of cells that expresses CD45 and CD56 is at least about 1% to about 12%. In some embodiments, the portion of the population of cells that expresses CD45 and CD56 is at least about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 11%, about 1% to about 12%, about 2% to about 3%, about 2% to about 4%, about 2% to about 5%, about 2% to about 6%, about 2% to about 7%, about 2% to about 8%, about 2% to about 9%, about 2% to about 10%, about 2% to about 11%, about 2% to about 12%, about 3% to about 4%, about 3% to about 5%, about 3% to about 6%, about 3% to about 7%, about 3% to about 8%, about 3% to about 9%, about 3% to about 10%, about 3% to about 11%, about 3% to about 12%, about 4% to about 5%, about 4% to about 6%, about 4% to about 7%, about 4% to about 8%, about 4% to about 9%, about 4% to about 10%, about 4% to about 11%, about 4% to about 12%, about 5% to about 6%, about 5% to about 7%, about 5% to about 8%, about 5% to about 9%, about 5% to about 10%, about 5% to about 11%, about 5% to about 12%, about 6% to about 7%, about 6% to about 8%, about 6% to about 9%, about 6% to about 10%, about 6% to about 11%, about 6% to about 12%, about 7% to about 8%, about 7% to about 9%, about 7% to about 10%, about 7% to about 11%, about 7% to about 12%, about 8% to about 9%, about 8% to about 10%, about 8% to about 11%, about 8% to about 12%, about 9% to about 10%, about 9% to about 11%, about 9% to about 12%, about 10% to about 11%, about 10% to about 12%, or about 11% to about 12%. In some embodiments, the portion of the population of cells that expresses CD45 and CD56 is at least about 1%, about 2%, about 3%, about 4%, about 5%, about 6%, about 7%, about 8%, about 9%, about 10%, about 11%, or about 12%. In some embodiments, the portion of the population of cells that expresses CD45 and CD56 is at least at least about 1%, about 2%, about 3%, about 4%, about 5%, about 6%, about 7%, about 8%, about 9%, about 10%, or about 11%. In some embodiments, the portion of the population of cells that expresses CD45 and CD56 is at least at most about 2%, about 3%, about 4%, about 5%, about 6%, about 7%, about 8%, about 9%, about 10%, about 11%, or about 12%.

In some embodiments, the portion of the population of cells that expresses CD45 and CD56 is at least about 15% to about 80%. In some embodiments, the portion of the population of cells that expresses CD45 and CD56 is at least about 15% to about 20%, about 15% to about 30%, about 15% to about 40%, about 15% to about 45%, about 15% to about 50%, about 15% to about 55%, about 15% to about 60%, about 15% to about 65%, about 15% to about 70%, about 15% to about 75%, about 15% to about 80%, about 20% to about 30%, about 20% to about 40%, about 20% to about 45%, about 20% to about 50%, about 20% to about 55%, about 20% to about 60%, about 20% to about 65%, about 20% to about 70%, about 20% to about 75%, about 20% to about 80%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 40% to about 45%, about 40% to about 50%, about 40% to about 55%, about 40% to about 60%, about 40% to about 65%, about 40% to about 70%, about 40% to about 75%, about 40% to about 80%, about 45% to about 50%, about 45% to about 55%, about 45% to about 60%, about 45% to about 65%, about 45% to about 70%, about 45% to about 75%, about 45% to about 80%, about 50% to about 55%, about 50% to about 60%, about 50% to about 65%, about 50% to about 70%, about 50% to about 75%, about 50% to about 80%, about 55% to about 60%, about 55% to about 65%, about 55% to about 70%, about 55% to about 75%, about 55% to about 80%, about 60% to about 65%, about 60% to about 70%, about 60% to about 75%, about 60% to about 80%, about 65% to about 70%, about 65% to about 75%, about 65% to about 80%, about 70% to about 75%, about 70% to about 80%, or about 75% to about 80%. In some embodiments, the portion of the population of cells that expresses CD45 and CD56 is at least about 15%, about 20%, about 30%, about 40%, about 45%, about 50%, about 55%, about 60%, about 65%, about 70%, about 75%, or about 80%. In some embodiments, the portion of the population of cells that expresses CD45 and CD56 is at least at least about 15%, about 20%, about 30%, about 40%, about 45%, about 50%, about 55%, about 60%, about 65%, about 70%, or about 75%. In some embodiments, the portion of the population of cells that expresses CD45 and CD56 is at least at most about 20%, about 30%, about 40%, about 45%, about 50%, about 55%, about 60%, about 65%, about 70%, about 75%, or about 80%. In some embodiments, the portion of the population of cells that expresses CD45 and CD56 is greater than 80%. In some embodiments, the portion of the population of cells that expresses CD45 and CD56 is anywhere in a range between 0.5% to greater than 90%.

### Double Positives

### CD14 and CD11b

The present disclosure provides a CD14 and CD11b-expressing cell or a cell that expresses any other immune-cell markers or markers associated with immune cell lineage disclosed herein comprising a nucleic acid comprising an open reading frame encoding one or more transcription factors, one or more transcription factors, or an activator of transcription of the open reading frame encoding one or more transcription factors. Transcription factors as disclosed herein may be exogenous transcription factors. In some embodiments, the immune cell is a macrophage/granulocyte lineage cell, including macrophages and granulocytes. In some embodiments, the immune cell is any immune cell expressing the immune cell markers or markers associated with immune cell lineage disclosed herein. In some embodiments, the CD14 and CD11b-expressing cell is a macrophage/granulocyte progenitor cell. In some embodiments, the CD14 and CD11b-expressing cell is a mature macrophage or granulocyte cell. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the CD14 and CD11b-expressing cell. In some embodiments, the CD14 and CD11b-expressing cell is provided in a media. The media may not have to be altered during the differentiation of the PSC into the CD14 and CD11b-expressing cell. In some embodiments, the nucleic acid comprising the open reading frame encoding the one or more transcription factors, the one or more transcription factors, or the activator of transcription of the open reading frame encoding one or more transcription factors induces the differentiation of the CD14 and CD11b-expressing cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides a population of cells comprising two or more CD14 and CD11b expressing cells as disclosed herein. At least one cell in the population of cells may express CD14 and CD11b. At least about 0.5% to at least about 5% of a population of cells may express CD14 and CD11b. In some embodiments, greater than 5% of a population of cells may express CD14 and CD11b. In some embodiments, the portion of the population of cells that expresses CD14 and CD11b is at least about 1% to about 5%. In some embodiments, the portion of the population of cells that expresses CD14 and CD11b is at least about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 2% to about 2.5%, about 2% to about 3%, about 2% to about 3.5%, about 2% to about 4%, about 2% to about 4.5%, about 2% to about 5%, about 2.5% to about 3%, about 2.5% to about 3.5%, about 2.5% to about 4%, about 2.5% to about 4.5%, about 2.5% to about 5%, about 3% to about 3.5%, about 3% to about 4%, about 3% to about 4.5%, about 3% to about 5%, about 3.5% to about 4%, about 3.5% to about 4.5%, about 3.5% to about 5%, about 4% to about 4.5%, about 4% to about 5%, or about 4.5% to about 5%. In some embodiments, the portion of the population of cells that expresses CD14 and CD11b is at least about 1%, about 1.5%, about 2%, about 2.5%, about 3%, about 3.5%, about 4%, about 4.5%, or about 5%. In some embodiments, the portion of the population of cells that expresses CD14 and CD11b is at least at least about 1%, about 1.5%, about 2%, about 2.5%, about 3%, about 3.5%, about 4%, or about 4.5%. In some embodiments, the portion of the population of cells that expresses CD14 and CD11b is at least at most about 1.5%, about 2%, about 2.5%, about 3%, about 3.5%, about 4%, about 4.5%, or about 5%. In some embodiments, the portion of the population of cells that expresses CD14 and CD11b is less than 1%. In some embodiments, the portion of the population of cells that expresses CD14 and CD11b is greater than 5%.

### T Cells

The present disclosure provides a T cell comprising a nucleic acid comprising an open reading frame encoding one or more transcription factors, one or more transcription factors, or an activator of transcription of the open reading frame encoding one or more transcription factors. Transcription factors as disclosed herein may be exogenous transcription factors. In some embodiments, the T cell is a regulatory T cell. In some embodiments, the T cell is a progenitor T cell. In some embodiments, the T cell is a mature T cell. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the T cell. In some embodiments, the T cell is provided in a media. The media may not have to be altered during the differentiation of the PSC into the T cell. In some embodiments, the nucleic acid comprising the open reading frame encoding the one or more transcription factors, the one or more transcription factors, or the activator of transcription of the open reading frame encoding one or more transcription factors induces the differentiation of the T cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 4 days or less, 1 day or less.

### T Cell Progenitor

The present disclosure provides a T cell progenitor cell comprising a nucleic acid comprising an open reading frame encoding one or more transcription factors, one or more transcription factors, or an activator of transcription of the open reading frame encoding one or more transcription factors. Transcription factors as disclosed herein may be exogenous transcription factors. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the T cell progenitor cell. In some embodiments, the T cell progenitor cell is provided in a media. The media may not have to be altered during the differentiation of the PSC into the T cell progenitor cell. In some embodiments, the nucleic acid comprising the open reading frame encoding the one or more transcription factors, the one or more transcription factors, or the activator of transcription of the open reading frame encoding one or more transcription factors induces the differentiation of the T cell progenitor cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 4 days or less, 1 day or less.

The present disclosure provides a population of cells comprising two or more progenitor T cells as disclosed herein. At least one cell in the population of cells may express CD45. At least about 0.5% to at least about 20% of a population of cells may express CD45. At least about 0.5%, 1%, 2%, 3%, 4%, 5%, 6%, 7%, 8%, 9%, 10, 11%, 12%, 13%, 14%, 15%, 16%, 17%, 18%, 19%, 20%, or more of a population of cells may express CD45. In some embodiments, at least about 1% to at least about 6% of a population of cells may express CD45. In some embodiments, at least about 3% to at least about 9% of a population of cells may express CD45.

The present disclosure provides a population of cells comprising two or more progenitor T cells as disclosed herein. At least one cell in the population of cells may express CD34. At least about 0.5% to at least about 20% of a population of cells may express CD45. At least about 0.5%, 1%, 2%, 3%, 4%, 5%, 6%, 7%, 8%, 9%, 10%, 11%, 12%, 13%, 14%, 15%, 16%, 17%, 18%, 19%, 20%, or more of a population of cells may express CD34. In some embodiments, at least about 2% to at least about 2.5% of a population of cells may express CD34.

### Mature T Cell

The present disclosure provides a mature T cell comprising a nucleic acid comprising an open reading frame encoding one or more transcription factors, one or more transcription factors, or an activator of transcription of the open reading frame encoding one or more transcription factors. Transcription factors as disclosed herein may be exogenous transcription factors. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the mature T cell. In some embodiments, the mature T cell is provided in a media. The media may not have to be altered during the differentiation of the PSC into the mature T cell. In some embodiments, the nucleic acid comprising the open reading frame encoding the one or more transcription factors, the one or more transcription factors, or the activator of transcription of the open reading frame encoding one or more transcription factors induces the differentiation of the mature T cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 4 days or less, 1 day or less.

The present disclosure provides a population of cells comprising two or more mature T cells as disclosed herein. At least one cell in the population of cells may express CD45. At least about 0.5% to at least about 20% of a population of cells may express CD45. At least about 0.5%, 1%, 2%, 3%, 4%, 5%, 6%, 7%, 8%, 9%, 10%, 11%, 12%, 13%, 14%, 15%, 16%, 17%, 18%, 19%, 20%, or more of a population of cells may express CD45. In some embodiments, at least about 1% to at least about 6% of a population of cells may express CD45. In some embodiments, at least about 3% to at least about 9% of a population of cells may express CD45.

The present disclosure provides a population of cells comprising two or more mature T cells as disclosed herein. At least one cell in the population of cells may express CD34. At least about 0.5% to at least about 20% of a population of cells may express CD45. At least about 0.5%, 1%, 2%, 3%, 4%, 5%, 6%, 7%, 8%, 9%, 10%, 11%, 12%, 13%, 14%, 15%, 16%, 17%, 18%, 19%, 20%, or more of a population of cells may express CD34. In some embodiments, at least about 2% to at least about 2.5% of a population of cells may express CD34.

### Transcription Factors

The present disclosure provides transcription factors and cells for inducing the differentiation of a pluripotent stem cell (PSC) into an immune cell.

Transcription factors (TFs) may be used to trigger differentiation programs. Certain transcription factors may be able to induce stem cells to differentiate to phenotypes. The phenotype may be an immune cell. Certain transcription factors may be able to induce stem cells to particular lineages, such as hematopoietic stem/progenitor cells, NK cells, regulatory T cells, FOXP3-expressing cells, progenitor T cells, CD34-expressing cells, CD45-expresisng cells, CD4-expressing cells, and mature T cells. In some aspects, combinations of transcription factors may be used to achieve differentiation to a particular cell lineage. The combinations may achieve a cell type or a cell sub-type that is not achieved by either transcription factor alone. The combination may achieve the same cell type as one of the transcription factors alone but may achieve it more efficiently.

The present disclosure provides an immune cell. The immune cell may comprise a nucleic acid comprising an open reading frame encoding one or more transcription factors. The nucleic acid may comprise two or more open reading frames encoding one or more transcription factors. The immune cell may comprise one or more transcription factors. The immune cell may comprise an activator of transcription of the open reading frame encoding one or more transcription factors. Transcription factors as disclosed herein may be exogenous transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the nucleic acid may comprise two or more open reading frames encoding one or more transcription factors. In some embodiments, the nucleic acid may comprise three or more open reading frames encoding one or more transcription factors. In some embodiments, the nucleic acid may comprise four or more open reading frames encoding one or more transcription factors. In some embodiments, the nucleic acid may comprise five or more open reading frames encoding one or more transcription factors. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less. In some embodiments, the one or more transcription factors comprise any one of the transcription factor combinations in Tables 1, 2, 3, 5, 6, 7, 8, 9, 10, or 11.

Some transcription factors may require a critical amount of expression to effectively induce differentiation, such as the equivalent of at least 5, 10, 15, 20, 25, or 50 copies of the open reading frame (ORF) per cell. Other factors may require less than a certain threshold of expression due to possible toxicity at high levels, such as less than 20, 10, or 5 copies per cell. Increased levels of expression may also be achieved by increasing the copy number of the ORF, for example, by using a higher copy number vector or by using a transposon.

PSCs comprising a nucleic acid comprising the open reading frame encoding the one or more transcription factors, one or more transcription factors, or an activator of transcription of the open reading frame encoding one or more transcription factors as disclosed herein may have a higher level of expression of one or more transcription factors compared to PSCs without the nucleic acid comprising the open reading frame encoding the one or more transcription factors, the one or more transcription factors, or the activator of transcription of the open reading frame encoding one or more transcription factors as disclosed herein. PSCs comprising a nucleic acid comprising the open reading frame encoding the one or more transcription factors, one or more transcription factors, or an activator of transcription of the open reading frame encoding one or more transcription factors as disclosed herein may have higher level of mRNA expression of one or more transcription factors compared to PSCs without the nucleic acid comprising the open reading frame encoding the one or more transcription factors, the one or more transcription factors, or the activator of transcription of the open reading frame encoding one or more transcription factors as disclosed herein. PSCs comprising a nucleic acid comprising the open reading frame encoding the one or more transcription factors, one or more transcription factors, or an activator of transcription of the open reading frame encoding one or more transcription factors as disclosed herein may have at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or greater expression of one or more transcription factors compared to PSCs without the nucleic acid comprising the open reading frame encoding one or more transcription factors, the one or more transcription factors, or the activator of transcription of the open reading frame encoding one or more transcription factors as disclosed herein.

### Zinc Fingers C2H2-Type

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be zinc fingers C2H2-type transcription factors. The zinc fingers C2H2-type transcription factor may be IKZF1, IKFZ4, KLF10, or ZBTB7B.

### IKZF1

The present disclosure provides immune cells and PSCs. The present disclosure provides an immune cell or PSC comprising a nucleic acid comprising an open reading frame encoding one or more transcription factors. The present disclosure provides an immune cell or PSC comprising one or more transcription factors. The present disclosure provides an immune cell or PSC comprising an activator of transcription of the open reading frame encoding one or more transcription factors, wherein the nucleic acid. The one or more transcription factors may be zinc fingers C2H2-type transcription factors. The zinc fingers C2H2-type transcription factor may be IKZF1.

IKZF1 may also be known as BAF Chromatin Remodeling Complex Subunit IKZF1, CTIP2, HRIT1-Alpha, CTIP-2, Radiation-Induced Tumor Suppressor Gene 1 Protein, B-Cell CLL/Lymphoma 11B (Zinc Finger Protein), IKZF1, BAF Complex Component, COUP-TF-Interacting Protein 2, B-Cell Lymphoma/Leukemia 111B, B Cell CLL/Lymphoma 111B, SMARCM2, ZNF856B, BCL-11B, HRit1, RIT1, B-Cell CLL/Lymphoma 11B/T-Cell Receptor Delta Constant Region Fusion Protein, Zinc Finger Protein HRit1 Alpha, B-Cell Lymphoma/Leukemia 11B, B-Cell CLL/Lymphoma 11B, IKZF1/TRDC Fusion, ATL1-Alpha, ATL1-Delta, ATL1-Gamma, ATL1-Beta, IDDFSTA, IMD49, and ATL1.

The present disclosure provides an expression cassette comprising one or more transcription factors. The one or more transcription factors may comprise IKZF1. IKZF1 may be introduced in an expression cassette. IKZF1 may be expressed in an expression cassette. IKZF1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising IKZF1, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise IKZF1. An expression cassette comprising IKZF1 may be introduced in a PSC. An expression cassette comprising IKZF1 may be expressed in a PSC. An expression cassette comprising IKZF1 may induce differentiation of the PSC into a Treg or a FOXP3-expressing cell. Different amounts of IKZF1 may be introduced into the PSC. At least 5, 10, 15, 20, 25, or 50 copies of the open reading frame (ORF) for IKZF1 per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or an activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise IKZF1. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise IKZF1. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise IKZF1. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise IKZF1. The one or more CD4-expressing cells may be differentiated from one or more PSCs. The one or more CD4-expressing cells may comprise one or more transcription factors that may induce differentiation of the one or more PSCs into the one or more CD4-expressing cells.

### IKZF2

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be zinc fingers C2H2-type transcription factors. The zinc fingers C2H2-type transcription factor may be, for example, IKZF2.

IKZF2 may also be known as IKAROS Family Zinc Finger 2, ZNFN1A2, Zinc Finger Protein, Subfamily 1A, 2 (Helios), Ikaros Family Zinc Finger Protein 2, Zinc Finger Protein Helios, Helios, Zinc Finger DNA Binding Protein Helios, IKAROS Family Zinc Finger 2 (Helios), ANF1A2, or ZNF1A2.

The present disclosure provides an expression cassette comprising one or more transcription factors. The one or more transcription factors may comprise IKZF2. IKZF2 may be introduced in an expression cassette. IKZF2 may be expressed in an expression cassette. IKZF2 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising IKZF2, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise IKZF2. An expression cassette comprising IKZF2 may be introduced in a PSC. An expression cassette comprising IKZF2 may be expressed in a PSC. An expression cassette comprising IKZF2 may induce differentiation of the PSC into a Treg or a FOXP3-expressing cell. Different amounts of IKZF2 may be introduced into the PSC. At least 5, 10, 15, 20, 25, or 50 copies of the open reading frame (ORF) for IKZF2 per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise IKZF2. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise IKZF2. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise IKZF2. The one or more CD34-expressing cells may be differentiated from one or more PSCs. The one or more CD34-expressing cells may comprise one or more transcription factors that may induce differentiation of the one or more PSCs into the one or more CD34-expressing cells. In some embodiments, a cell expressing immune cell marker such as for example, CD4, or CD8, or CD56, or any other marker associated with immune-cell lineage, or an immune cell marker disclosed herein may comprise one or more transcription factors that may induce differentiation of the one or more PSCs into the one or more immune cell marker-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise IKZF2. The one or more CD4-expressing cells may be differentiated from one or more PSCs. The one or more CD4-expressing cells may comprise one or more transcription factors that may induce differentiation of the one or more PSCs into the one or more CD4-expressing cells.

### IKZF4

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be zinc fingers C2H2-type transcription factors. The zinc fingers C2H2-type transcription factor may be, for example, IKZF4.

IKZF4 may also be known as IKAROS Family Zinc Finger 4, ZNFN1A4, Zinc Finger Protein, Subfamily 1A, 4 (Eos), Ikaros Family Zinc Finger Protein 4, Zinc Finger Protein Eos, Eos, Zinc Finger Transcription Factor Eos, IKAROS Family Zinc Finger 4 (Eos), KIAA1782, or EOS.

The present disclosure provides an expression cassette comprising one or more transcription factors. The one or more transcription factors may comprise IKZF4. IKZF4 may be introduced in an expression cassette. IKZF4 may be expressed in an expression cassette. IKZF4 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising IKZF4, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise IKZF4. An expression cassette comprising IKZF4 may be introduced in a PSC. An expression cassette comprising IKZF4 may be expressed in a PSC. An expression cassette comprising IKZF4 may induce differentiation of the PSC into a Treg or a FOXP3-expressing cell. Different amounts of IKZF4 may be introduced into the PSC. At least 5, 10, 15, 20, 25, or 50 copies of the open reading frame (ORF) for IKZF4 per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise IKZF4. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise IKZF4. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise IKZF4. The one or more CD34-expressing cells may be differentiated from one or more PSCs. The one or more CD34-expressing cells may comprise one or more transcription factors that may induce differentiation of the one or more PSCs into the one or more CD34-expressing cells. In some embodiments, a cell expressing immune cell marker such as for example, CD4, or CD8, or CD56, or any other marker associated with immune-cell lineage, or an immune cell marker disclosed herein may comprise one or more transcription factors that may induce differentiation of the one or more PSCs into the one or more immune cell marker-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise IKZF4. The one or more CD4-expressing cells may be differentiated from one or more PSCs. The one or more CD4-expressing cells may comprise one or more transcription factors that may induce differentiation of the one or more PSCs into the one or more CD4-expressing cells.

### KLF10

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be zinc fingers C2H2-type transcription factors. The zinc fingers C2H2-type transcription factor may be, for example, KLF10.

KLF10 may also be known as Kruppel Like Factor 10, TIEG1, EGRA, TIEG, Transforming Growth Factor-Beta-Inducible Early Growth Response Protein 1, TGFB-Inducible Early Growth Response Protein 1, Krueppel-Like Factor 10, EGR-Alpha, Zinc Finger Transcription Factor TIEG, TGFB Inducible Early Growth Response, Early Growth Response Alpha, or TIEG-1.

The present disclosure provides an expression cassette comprising one or more transcription factors. The one or more transcription factors may comprise KLF10. KLF10 may be introduced in an expression cassette. KLF10 may be expressed in an expression cassette. KLF10 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising KLF10, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise KLF10. An expression cassette comprising KLF10 may be introduced in a PSC. An expression cassette comprising KLF10 may be expressed in a PSC. An expression cassette comprising KLF10 may induce differentiation of the PSC into a Treg or a FOXP3-expressing cell. Different amounts of KLF10 may be introduced into the PSC. At least 5, 10, 15, 20, 25, or 50 copies of the open reading frame (ORF) for KLF10 per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise KLF10. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise KLF10. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise KLF10. The one or more CD34-expressing cells may be differentiated from one or more PSCs. The one or more CD34-expressing cells may comprise one or more transcription factors that may induce differentiation of the one or more PSCs into the one or more CD34-expressing cells. In some embodiments, a cell expressing immune cell marker such as for example, CD4, or CD8, or CD56, or any other marker associated with immune-cell lineage, or an immune cell marker disclosed herein may comprise one or more transcription factors that may induce differentiation of the one or more PSCs into the one or more immune cell marker-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise KLF10. The one or more CD4-expressing cells may be differentiated from one or more PSCs. The one or more CD4-expressing cells may comprise one or more transcription factors that may induce differentiation of the one or more PSCs into the one or more CD4-expressing cells.

### ZBTB7B

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be zinc fingers C2H2-type transcription factors. The zinc fingers C2H2-type transcription factor may be, for example, ZBTB7B.

ZBTB7B may also be known as Zinc Finger And BTB Domain Containing 7B, ZNF857B, ZBTB15, HcKrox, ZFP67, Zinc Finger And BTB Domain-Containing Protein 7B, T-Helper-Inducing POZ/Krueppel-Like Factor, Krueppel-Related Zinc Finger Protein CKrox, Zinc Finger And BTB Domain Containing 15, Zinc Finger Protein 67 Homolog, Zinc Finger Protein Th-POK, Zinc Finger Protein 857B, C-Krox, Zinc Finger And BTB Domain-Containing Protein 15, Zinc Finger Protein 67 Homolog (Mouse), ZFP-67, C-KROX, HcKROX, Zfp-67, CKROX, or THPOK.

The present disclosure provides an expression cassette comprising one or more transcription factors. The one or more transcription factors may comprise ZBTB7B. ZBTB7B may be introduced in an expression cassette. ZBTB7B may be expressed in an expression cassette. ZBTB7B may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising ZBTB7B, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise ZBTB7B. An expression cassette comprising ZBTB7B may be introduced in a PSC. An expression cassette comprising ZBTB7B may be expressed in a PSC. An expression cassette comprising ZBTB7B may induce differentiation of the PSC into a Treg or a FOXP3-expressing cell. Different amounts of ZBTB7B may be introduced into the PSC. At least 5, 10, 15, 20, 25, or 50 copies of the open reading frame (ORF) for ZBTB7B per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise ZBTB7B. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise ZBTB7B. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise ZBTB7B. The one or more CD34-expressing cells may be differentiated from one or more PSCs. The one or more CD34-expressing cells may comprise one or more transcription factors that may induce differentiation of the one or more PSCs into the one or more CD34-expressing cells. In some embodiments, a cell expressing immune cell marker such as for example, CD4, or CD8, or CD56, or any other marker associated with immune-cell lineage, or an immune cell marker disclosed herein may comprise one or more transcription factors that may induce differentiation of the one or more PSCs into the one or more immune cell marker-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise ZBTB7B. The one or more CD4-expressing cells may be differentiated from one or more PSCs. The one or more CD4-expressing cells may comprise one or more transcription factors that may induce differentiation of the one or more PSCs into the one or more CD4-expressing cells.

### KLF1

The present disclosure provides immune cells and PSCs. The present disclosure provides an immune cell or PSC comprising a nucleic acid comprising an open reading frame encoding one or more transcription factors. The present disclosure provides an immune cell or PSC comprising one or more transcription factors. The present disclosure provides an immune cell or PSC comprising an activator of transcription of the open reading frame encoding one or more transcription factors, wherein the nucleic acid. The one or more transcription factors may be zinc fingers C2H2-type transcription factors. The zinc fingers C2H2-type transcription factor may be KLF1.

KLF1 may also be known as EKLF, Erythroid Krueppel-like Transcription Factor, Erythroid Kruppel-Like Factor, Krueppel-Like Factor 1, Kruppel Like Factor 1, Erythroid-Specific Transcription Factor EKLF, Kruppel-Like Factor 1 (Erythroid), EKLF/KLF1.

The present disclosure provides an expression cassette comprising one or more transcription factors. The one or more transcription factors may comprise KLF1. KLF1 may be introduced in an expression cassette. KLF1 may be expressed in an expression cassette. KLF1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising KLF1, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise KLF1. An expression cassette comprising KLF1 may be introduced in a PSC. An expression cassette comprising KLF1 may be expressed in a PSC. An expression cassette comprising KLF1 may induce differentiation of the PSC into a Treg or a FOXP3-expressing cell. Different amounts of KLF1 may be introduced into the PSC. At least 5, 10, 15, 20, 25, or 50 copies of the open reading frame (ORF) for KLF1 per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or an activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, a cell expressing immune cell marker such as for example, CD4, or CD8, or CD56, or any other marker associated with immune-cell lineage, or an immune cell marker disclosed herein may comprise one or more transcription factors that may induce differentiation of the one or more PSCs into the one or more immune cell marker-expressing cells. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise KLF1. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise KLF1. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise KLF1. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise KLF1. The one or more CD4-expressing cells may be differentiated from one or more PSCs. The one or more CD4-expressing or CD8-expressing cells may comprise one or more transcription factors that may induce differentiation of the one or more PSCs into the one or more CD4-expressing cells.

### MADS Box Transcription Enhancer Factor 2 Family

### MEF2C

This locus encodes a member of the MADS box transcription enhancer factor 2 (MEF2) family of proteins, which play a role in myogenesis. The encoded protein, MEF2 polypeptide C, has both trans-activating and DNA binding activities. MEF2C (Myocyte Enhancer Factor 2C) is a protein coding gene.

The present disclosure provides immune cells and PSCs. The present disclosure provides an immune cell or PSC comprising a nucleic acid comprising an open reading frame encoding one or more transcription factors. The present disclosure provides an immune cell or PSC comprising one or more transcription factors. The present disclosure provides an immune cell or PSC comprising an activator of transcription of the open reading frame encoding one or more transcription factors, wherein the nucleic acid. The one or more transcription factors may be MADS box transcription enhancer factor 2 (MEF2) family of proteins. The MADS box transcription enhancer factor 2 family of proteins transcription factor may be MEF2C.

Myocyte Enhancer Factor 2C (MEF2C) may also be known as Myocyte-Specific Enhancer Factor 2C, MADS Box Transcription Enhancer Factor 2, Polypeptide C, C5DELq14.3, DEL5q14.3, or NEDHSIL.

The present disclosure provides an expression cassette comprising one or more transcription factors. The one or more transcription factors may comprise MEF2C. MEF2C may be introduced in an expression cassette. MEF2C may be expressed in an expression cassette. MEF2C may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising MEF2C, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise MEF2C. An expression cassette comprising MEF2C may be introduced in a PSC. An expression cassette comprising MEF2C may be expressed in a PSC. An expression cassette comprising MEF2C may induce differentiation of the PSC into a Treg or a FOXP3-expressing cell. Different amounts of MEF2C may be introduced into the PSC. At least 5, 10, 15, 20, 25, or 50 copies of the open reading frame (ORF) for MEF2C per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or an activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45 In some embodiments, a cell expressing immune cell marker such as for example, CD4, or CD8, or CD56, or any other marker associated with immune-cell lineage, or an immune cell marker disclosed herein may comprise one or more transcription factors that may induce differentiation of the one or more PSCs into the one or more immune cell marker-expressing cells. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise MEF2C. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise MEF2C. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise MEF2C. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise MEF2C. The one or more CD4-expressing cells may be differentiated from one or more PSCs. The one or more CD4-expressing or CD8-expressing cells may comprise one or more transcription factors that may induce differentiation of the one or more PSCs into the one or more CD4-expressing cells.

### JUN Family

### JUN/AP-1

The present disclosure provides cells, expression cassettes, and methods using transcription factors, or molecules that increase transcription or increase activation of transcription factors. One or more transcription factors may be used. A transcription factor may be a member of the JUN family. The present disclosure provides immune cells and PSCs. The present disclosure provides an immune cell or PSC comprising a nucleic acid comprising an open reading frame encoding one or more transcription factors. The present disclosure provides an immune cell or PSC comprising one or more transcription factors. The present disclosure provides an immune cell or PSC comprising an activator of transcription of the open reading frame encoding one or more transcription factors, wherein the nucleic acid. The one or more transcription factors may be JUN family of proteins. The JUN family of proteins transcription factor may be JUN/AP-1 (JUN). The transcription factor may be JUN/AP-1. JUN/AP-1 may also be known as Jun Proto-Oncogene, AP-1 Transcription Factor Subunit; V-Jun Avian Sarcoma Virus 17 Oncogene Homolog; C-Jun; AP-1; Transcription Factor AP-1; Proto-Oncogene C-Jun; Activator Protein 1; Jun Oncogene; AP1; P39; V-Jun Sarcoma Virus 17 Oncogene Homolog; Jun Activation Domain Binding Protein; Enhancer-Binding Protein AP1; Proto-Oncogene C-Jun; or C-JUN. In the present disclosure JUN/AP-1 may referred to interchangeably, as JUN or AP-1 or JUN/AP-1.

The present disclosure provides an expression cassette comprising one or more transcription factors. The one or more transcription factors may comprise JUN/AP-1. JUN/AP-1 may be introduced in an expression cassette. JUN/AP-1 may be expressed in an expression cassette. JUN/AP-1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising JUN/AP-1 and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise JUN/AP-1. An expression cassette comprising JUN/AP-1 may be introduced in a PSC. An expression cassette comprising JUN/AP-1 may be expressed in a PSC. An expression cassette comprising JUN/AP-1 may induce differentiation of the PSC into a Treg or a FOXP3-expressing cell. Different amounts of JUN/AP-1 may be introduced into the PSC. At least 5, 10, 15, 20, 25, or 50 copies of the open reading frame (ORF) for JUN/AP-1 per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or an activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, a cell expressing immune cell marker such as for example, CD4, or CD8, or CD56, or any other marker associated with immune-cell lineage, or an immune cell marker disclosed herein may comprise one or more transcription factors that may induce differentiation of the one or more PSCs into the one or more immune cell marker-expressing cells. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise JUN/AP-1. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise JUN/AP-1. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise JUN/AP-1. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise JUN/AP-1. The one or more CD4-expressing cells may be differentiated from one or more PSCs. The one or more CD4-expressing or CD8-expressing cells may comprise one or more transcription factors that may induce differentiation of the one or more PSCs into the one or more CD4-expressing cells.

### Forkhead Box

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be forkhead box transcription factors. The forkhead box transcription factor may be FOXO1.

### FOXO1

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be forkhead box transcription factors. The forkhead box transcription factor may be FOXO1.

FOXO1 may also be known as Forkhead Box O1, FOXO1A, FKH1, FKHR, Forkhead Box Protein O1A, Forkhead Box Protein O1, Forkhead, Drosophila, Homolog Of, In Rhabdomyosarcoma, Forkhead Homolog In Rhabdomyosarcoma, or Forkhead In Rhabdomyosarcoma.

The present disclosure provides an expression cassette comprising one or more transcription factors. The one or more transcription factors may comprise FOXO1. FOXO1 may be introduced in an expression cassette. FOXO1 may be expressed in an expression cassette. FOXO1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising FOXO1, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise FOXO1. An expression cassette comprising FOXO1 may be introduced in a PSC. An expression cassette comprising FOXO1 may be expressed in a PSC. An expression cassette comprising FOXO1 may induce differentiation of the PSC into a Treg or a FOXP3-expressing cell. Different amounts of FOXO1 may be introduced into the PSC. At least 5, 10, 15, 20, 25, or 50 copies of the open reading frame (ORF) for FOXO1 per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise FOXO1. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise FOXO1. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise FOXO1. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise FOXO1. The one or more CD4-expressing cells may be differentiated from one or more PSCs. The one or more CD4-expressing cells may comprise one or more transcription factors that may induce differentiation of the one or more PSCs into the one or more CD4-expressing cells.

### FOXO3

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be forkhead box transcription factors. The forkhead box transcription factor may be FOXO3.

FOXO3 may also be known as Forkhead Box 03, AF6q21, FKHRL1, FOXO3A, FOXO2, Forkhead In Rhabdomyosarcoma-Like 1, Forkhead Box Protein 03, Forkhead, Drosophila, Homolog Of, In Rhabdomyosarcoma-Like 1, Forkhead Homolog (Rhabdomyosarcoma) Like 1, Forkhead Box 03A, AF6q21 Protein, FKHRL1P2, or FOXO3A.

The present disclosure provides an expression cassette comprising one or more transcription factors. The one or more transcription factors may comprise FOXO3. FOXO3 may be introduced in an expression cassette. FOXO3 may be expressed in an expression cassette. FOXO3 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising FOXO3, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise FOXO3. An expression cassette comprising FOXO3 may be introduced in a PSC. An expression cassette comprising FOXO3 may be expressed in a PSC. An expression cassette comprising FOXO3 may induce differentiation of the PSC into a Treg or a FOXP3-expressing cell. Different amounts of FOXO3 may be introduced into the PSC. At least 5, 10, 15, 20, 25, or 50 copies of the open reading frame (ORF) for FOXO3 per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise FOXO3. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise FOXO3. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise FOXO3. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise FOXO3. The one or more CD4-expressing cells may be differentiated from one or more PSCs. The one or more CD4-expressing cells may comprise one or more transcription factors that may induce differentiation of the one or more PSCs into the one or more CD4-expressing cells.

### FOXP3

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be forkhead box transcription factors. The forkhead box transcription factor may be FOXP3.

FOXP3 may also be known as Forkhead Box P3, DIETER, XPID, AIID, PIDX, IPEX, JM2, Immune Dysregulation, Polyendocrinopathy, Enteropathy, X-Linked, Forkhead Box Protein P3, SCURFIN, Immunodeficiency, Polyendocrinopathy, Enteropathy, X-Linked, FOXP3delta7, or Scurfin.

The present disclosure provides an expression cassette comprising one or more transcription factors. The one or more transcription factors may comprise FOXP3. FOXP3 may be introduced in an expression cassette. FOXP3 may be expressed in an expression cassette. FOXP3 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising FOXP3, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise FOXP3. An expression cassette comprising FOXP3 may be introduced in a PSC. An expression cassette comprising FOXP3 may be expressed in a PSC. An expression cassette comprising FOXP3 may induce differentiation of the PSC into a Treg or a FOXP3-expressing cell. Different amounts of FOXP3 may be introduced into the PSC. At least 5, 10, 15, 20, 25, or 50 copies of the open reading frame (ORF) for FOXP3 per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise FOXP3. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise FOXP3. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise FOXP3. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise FOXP3. The one or more CD4-expressing cells may be differentiated from one or more PSCs. The one or more CD4-expressing cells may comprise one or more transcription factors that may induce differentiation of the one or more PSCs into the one or more CD4-expressing cells.

### NF-κB Transcription Factor Family

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be NF-κB transcription factor. The NF-κB transcription factor may be NFKB1.

### NFKB1

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be NF-κB transcription factors. The NF-κB transcription factors may be, for example, NFKB1.

NFKB1 may also be known as Nuclear Factor Kappa B Subunit, Nuclear Factor Of Kappa Light Polypeptide Gene Enhancer In B-Cells, Nuclear Factor NF-Kappa-B P105 Subunit, NF-KappaB, NFKB-P50, NFkappaB, NF-KB1, KBF1, DNA-Binding Factor KBF1, EBP-1, P105, P50, Nuclear Factor Kappa-B DNA Binding Subunit, Nuclear Factor NF-Kappa-B P50 Subunit, NF-Kappabeta, NF-Kappa-B1, NFKB-P105, and CVID12 NF-KB.

The present disclosure provides an expression cassette comprising one or more transcription factors. The one or more transcription factors may comprise NFKB1. NFKB1 may be introduced in an expression cassette. NFKB1 may be expressed in an expression cassette. NFKB1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising NFKB1, and one or more other transcription factors may induce differentiation of the PSC into a Treg or a NFKB1-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise NFKB1. An expression cassette comprising NFKB1 may be introduced in a PSC. An expression cassette comprising NFKB1 may be expressed in a PSC. An expression cassette comprising NFKB1 may induce differentiation of the PSC into a Treg or a NFKB1-expressing cell. Different amounts of NFKB1 may be introduced into the PSC. At least 5, 10, 15, 20, 25, or 50 copies of the open reading frame (ORF) for NFKB1 per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses NFKB1. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NFKB1. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NFKB1. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NFKB1. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NFKB1. The one or more CD4-expressing cells may be differentiated from one or more PSCs. The one or more CD4-expressing cells may comprise one or more transcription factors that may induce differentiation of the one or more PSCs into the one or more CD4-expressing cells.

### REL

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be NF-κB transcription factors. The NF-κB transcription factors may be, for example, REL.

REL may also be known as REL Proto-Oncogene, NF-κB Subunit, HIVEN86A, C-Rel, V-Rel Avian Reticuloendotheliosis Viral Oncogene Homolog, Proto-Oncogene C-Rel, I-Rel, Oncogene REL, Avian Reticuloendotheliosis, or IMD92.

The present disclosure provides an expression cassette comprising one or more transcription factors. The one or more transcription factors may comprise REL. REL may be introduced in an expression cassette. REL may be expressed in an expression cassette. REL may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising REL, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise REL. An expression cassette comprising REL may be introduced in a PSC. An expression cassette comprising REL may be expressed in a PSC. An expression cassette comprising REL may induce differentiation of the PSC into a Treg or a FOXP3-expressing cell. Different amounts of REL may be introduced into the PSC. At least 5, 10, 15, 20, 25, or 50 copies of the open reading frame (ORF) for REL per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses FOXP3. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise REL. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise REL. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise REL. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise REL. The one or more CD4-expressing cells may be differentiated from one or more PSCs. The one or more CD4-expressing cells may comprise one or more transcription factors that may induce differentiation of the one or more PSCs into the one or more CD4-expressing cells.

### ETS-Domain Transcription Factor

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be ETS-domain transcription factors. The ETS-domain transcription factor may be, for example, GABPA.

### GABPA

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be GABPA. GABPA may also be known as GA Binding Protein Transcription Factor Subunit Alpha, E4TF1A, E4TF1-60, NRF2A, NFT2, NRF2, Human Nuclear Respiratory Factor-2 Subunit Alpha, Nuclear Respiratory Factor 2 Alpha Subunit, Nuclear Respiratory Factor 2 Subunit Alpha, GA-Binding Protein Alpha Chain, Transcription Factor E4TF1-60, GABP Subunit Alpha, GA-Binding Protein Transcription Factor, Alpha Subunit (60kD), GA Binding Protein Transcription Factor, Alpha Subunit 60 kDa, GA Binding Protein Transcription Factor Alpha Subunit 60 kDa, GA Binding Protein Transcription Factor Alpha Subunit, and RCH04A07.

The present disclosure provides an expression cassette comprising one or more transcription factors. The one or more transcription factors may comprise GABPA. GABPA may be introduced in an expression cassette. GABPA may be expressed in an expression cassette. GABPA may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising GABPA, and one or more other transcription factors may induce differentiation of the PSC into a Treg or a GABPA-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise GABPA. An expression cassette comprising GABPA may be introduced in a PSC. An expression cassette comprising GABPA may be expressed in a PSC. An expression cassette comprising GABPA may induce differentiation of the PSC into a Treg or a FOXP3-expressing cell. Different amounts of GABPA may be introduced into the PSC. At least 5, 10, 15, 20, 25, or 50 copies of the open reading frame (ORF) for GABPA per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses GABPA. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise GABPA. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise GABPA. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise GABPA. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise GABPA. The one or more CD4-expressing cells may be differentiated from one or more PSCs. The one or more CD4-expressing cells may comprise one or more transcription factors that may induce differentiation of the one or more PSCs into the one or more CD4-expressing cells.

### Basic Helix-Loop-Helix Proteins

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be basic helix-loop-helix proteins. The basic helix-loop-helix protein may be TCF12 or TCF3.

### TCF12 or HEB

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be basic helix-loop-helix proteins. The basic helix-loop-helix protein may be TCF12 or HEB.

TCF12 may also be known as HEB, Transcription Factor 12, BHLHb20, HTF4HsT17266, P64, Class B Basic Helix-Loop-Helix Protein 20, Helix-Loop-Helix Transcription Factor 4, Transcription Factor HTF-4, DNA-Binding Protein HTF4, E-Box-Binding Protein, TCF-12, BHLHB20, CRS3, or HH26.

The present disclosure provides an expression cassette comprising one or more transcription factors. The one or more transcription factors may comprise TCF12. TCF12 may be introduced in an expression cassette. TCF12 may be expressed in an expression cassette. TCF12 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising TCF12, and one or more other transcription factors may induce differentiation of the PSC into a Treg or a TCF12-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise TCF12. An expression cassette comprising TCF12 may be introduced in a PSC. An expression cassette comprising TCF12 may be expressed in a PSC. An expression cassette comprising TCF12 may induce differentiation of the PSC into a Treg or a TCF12-expressing cell. Different amounts of TCF12 may be introduced into the PSC. At least 5, 10, 15, 20, 25, or 50 copies of the open reading frame (ORF) for TCF12 per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses TCF12. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TCF12. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TCF12. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TCF12. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TCF12. The one or more CD4-expressing cells may be differentiated from one or more PSCs. The one or more CD4-expressing cells may comprise one or more transcription factors that may induce differentiation of the one or more PSCs into the one or more CD4-expressing cells.

### TCF3

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be basic helix-loop-helix proteins. The basic helix-loop-helix protein may be TCF3.

TCF3 may also be known as BHLHb21, ITF1, E2A, Immunoglobulin Transcription Factor 1, Transcription Factor E2-Alpha, Kappa-E2-Binding Factor, VDIR, E47, P75, Class B Basic Helix-Loop-Helix Protein 21, Transcription Factor ITF-1, VDR Interacting Repressor, MGC129647, MGC129648, TCF-3, Transcription Factor 3 (E2A Immunoglobulin Enhancer Binding Factors E12/E47), Negative Vitamin D Response Element-Binding Protein, E2A Immunoglobulin Enhancer-Binding Factor E12/E47, Immunoglobulin Enhancer-Binding Factor E12/E47, Vitamin D Receptor-Interacting Repressor, E2A-HLF Fusion Transcript Protein, Helix-Loop-Helix Protein HE47, NOL1-TCF3 Fusion, BHLHB21, or AGM8.

The present disclosure provides an expression cassette comprising one or more transcription factors. The one or more transcription factors may comprise TCF3. TCF3 may be introduced in an expression cassette. TCF3 may be expressed in an expression cassette. TCF3 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising TCF3, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise TCF3. An expression cassette comprising TCF3 may be introduced in a PSC. An expression cassette comprising TCF3 may be expressed in a PSC. An expression cassette comprising TCF3 may induce differentiation of the PSC into a Treg or a TCF3-expressing cell. Different amounts of TCF3 may be introduced into the PSC. At least 5, 10, 15, 20, 25, or 50 copies of the open reading frame (ORF) for TCF3 per cell may be introduced. Increased levels of expression may also be achieved by increasing the copy number of the ORF, for example, by using a higher copy number vector or by using a transposon.

The present disclosure provides an immune cell comprising an open reading frame encoding one or more transcription factors, one or more transcription factors, or activator of transcription of the open reading frame encoding one or more transcription factors. In some embodiments, the immune cell is a T cell. In some embodiments, the immune cell is a hematopoietic stem/progenitor cell. In some embodiments, the immune cell is a NK cell. In some embodiments, the T cell is a regulatory T cell. In some embodiments, the immune cell is an earlier stage hematopoietic progenitor to a T cell. In some embodiments, the immune cell is a committed progenitor cell. In some embodiments, the immune cell is a mature T cell. In some embodiments, the immune cell expresses TCF3. In some embodiments, the immune cell expresses CD45. In some embodiments, the immune cell expresses CD34. In some embodiments, the immune cell expresses CD4. In some embodiments, the nucleic acid comprising an open reading frame encoding one or more transcription factors, the one or more transcription factors, or the activator of transcription of the open reading frame encoding one or more transcription factors is expressed to induce the differentiation of a PSC into the immune cell. In some embodiments, the immune cell is provided in a media. The media may not have to be altered during the differentiation of the PSC into the immune cell. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 28 days or less. In some embodiments, the nucleic acid comprising the open reading frame encoding the one or more transcription factors, the one or more transcription factors, or the activator of transcription of the open reading frame encoding one or more transcription factors induces the differentiation of the immune cell from the PSC in 11 days or less, 5 days or less, 4 days or less, 1 day or less.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TCF3. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TCF3. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TCF3. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TCF3. The one or more CD4-expressing cells may be differentiated from one or more PSCs. The one or more CD4-expressing cells may comprise one or more transcription factors that may induce differentiation of the one or more PSCs into the one or more CD4-expressing cells.

### Ring Finger Proteins

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be ring finger proteins. The ring finger protein may be, for example, DTX1.

### DTX1

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The transcription factor may be DTX1. DTX1 may also be known as Deltex E3 Ubiquitin Ligase 1, RNF140, HDx-1, RING-Type E3 Ubiquitin Transferase DTX1, E3 Ubiquitin-Protein Ligase DTX1, Protein Deltex-1, Deltex Homolog 1 (Drosophila), Deltex 1, E3 Ubiquitin Ligase, Deltex Homolog 1, EC 2.3.2.27, Deltex1, and HDTX1.

The present disclosure provides an expression cassette comprising one or more transcription factors. The one or more transcription factors may comprise DTX1. DTX1 may be introduced in an expression cassette. DTX1 may be expressed in an expression cassette. DTX1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising DTX1, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise DTX1. An expression cassette comprising DTX1 may be introduced in a PSC. An expression cassette comprising DTX1 may be expressed in a PSC. An expression cassette comprising DTX1 may induce differentiation of the PSC into a Treg or a FOXP3-expressing cell. Different amounts of DTX1 may be introduced into the PSC. At least 5, 10, 15, 20, 25, or 50 copies of the open reading frame (ORF) for DTX1 per cell may be introduced. Increased levels of expression may also be achieved by increasing the copy number of the ORF, for example, by using a higher copy number vector or by using a transposon.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise DTX1. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise DTX1. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise DTX1. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise DTX1. The one or more CD4-expressing cells may be differentiated from one or more PSCs. The one or more CD4-expressing cells may comprise one or more transcription factors that may induce differentiation of the one or more PSCs into the one or more CD4-expressing cells.

### GATA Zinc Finger Domain Containing

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be GATA zinc finger domain containing. The GATA zinc finger domain containing transcription factor may be, for example, GATA1 or GATA3.

### GATA1

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The transcription factor may be GATA1. GATA1 may also be known as GATA Binding Protein 1, GATA-1, ERYF1, NF-E1, NFE1, GF1, Erythroid Transcription Factor, Globin Transcription Factor 1, Nuclear Factor, Erythroid 1, NF-E1 DNA-Binding Protein, GATA-Binding Factor 1, GF-1, GATA-Binding Protein 1 (Globin Transcription Factor 1), Erythroid Transcription Factor 1, Transcription Factor GATA1, XLANP, XLTDA, Eryf1, and XLTT.

The present disclosure provides an expression cassette comprising one or more transcription factors. The one or more transcription factors may comprise GATA1. GATA1 may be introduced in an expression cassette. GATA1 may be expressed in an expression cassette. GATA1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising GATA1, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise GATA1. An expression cassette comprising GATA1 may be introduced in a PSC. An expression cassette comprising GATA1 may be expressed in a PSC. An expression cassette comprising GATA1 may induce differentiation of the PSC into a Treg or a FOXP3-expressing cell. Different amounts of GATA1 may be introduced into the PSC. At least 5, 10, 15, 20, 25, or 50 copies of the open reading frame (ORF) for GATA1 per cell may be introduced. Increased levels of expression may also be achieved by increasing the copy number of the ORF, for example, by using a higher copy number vector or by using a transposon.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise GATA1. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise GATA1. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise GATA1. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise GATA1. The one or more CD4-expressing cells may be differentiated from one or more PSCs. The one or more CD4-expressing cells may comprise one or more transcription factors that may induce differentiation of the one or more PSCs into the one or more CD4-expressing cells.

### GATA3

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The transcription factor may be GATA3. GATA3 may also be known as GATA Binding Protein 3, HDR, Trans-Acting T-Cell-Specific Transcription Factor GATA-3, GATA-Binding Factor 3, GATA-Binding Protein 3, or HDRS.

The present disclosure provides an expression cassette comprising one or more transcription factors. The one or more transcription factors may comprise GATA3. GATA3 may be introduced in an expression cassette. GATA3 may be expressed in an expression cassette. GATA3 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising GATA3, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise GATA3. An expression cassette comprising GATA3 may be introduced in a PSC. An expression cassette comprising GATA3 may be expressed in a PSC. An expression cassette comprising GATA3 may induce differentiation of the PSC into a Treg or a FOXP3-expressing cell. Different amounts of GATA3 may be introduced into the PSC. At least 5, 10, 15, 20, 25, or 50 copies of the open reading frame (ORF) for GATA3 per cell may be introduced. Increased levels of expression may also be achieved by increasing the copy number of the ORF, for example, by using a higher copy number vector or by using a transposon.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise GATA3. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise GATA3. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise GATA3. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise GATA3. The one or more CD4-expressing cells may be differentiated from one or more PSCs. The one or more CD4-expressing cells may comprise one or more transcription factors that may induce differentiation of the one or more PSCs into the one or more CD4-expressing cells.

### Runt-Related Transcription Factors

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be runt-related transcription factors. The runt-related transcription factor may be, for example, RUNX1.

### RUNX1

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The transcription factor may be RUNX1. RUNX1 may also be known as transcription factors may be used. A transcription factor may be RUNX1. RUNX1 may also be known as RUNX Family Transcription Factor, Runt-Related Transcription Factor, AMLCR1, CBFA2, AML1, Polyomavirus Enhancer-Binding Protein 2 Alpha B Subunit, SL3/AKV Core-Binding Factor Alpha B Subunit, SL3-3 Enhancer Factor 1 Alpha B Subunit, Runt Related Transcription Factor, Acute Myeloid Leukemia 1 Protein, Oncogene AML-1, PEBP2-Alpha B, PEA2-Alpha B, PEBP2A2, Core-Binding Factor, Runt Domain, Alpha Subunit, Core-Binding Factor Subunit Alpha-2, AML1-EVI-1 Fusion Protein, Acute Myeloid Leukemia 1, AML1-ETO Fusion Protein, AML1-ETO Fusion, Aml1 Oncogene, Mutant RUNX1, CBF-Alpha-2, AML1-EVI-1, PEBP2alpha, CBF2alpha, PEBP2aB, or EVI-1.

The present disclosure provides an expression cassette comprising one or more transcription factors. The one or more transcription factors may comprise RUNX1. RUNX1 may be introduced in an expression cassette. RUNX1 may be expressed in an expression cassette. RUNX1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising RUNX1, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise RUNX1. An expression cassette comprising RUNX1 may be introduced in a PSC. An expression cassette comprising RUNX1 may be expressed in a PSC. An expression cassette comprising RUNX1 may induce differentiation of the PSC into a Treg or a FOXP3-expressing cell. Different amounts of RUNX1 may be introduced into the PSC. At least 5, 10, 15, 20, 25, or 50 copies of the open reading frame (ORF) for RUNX1 per cell may be introduced. Increased levels of expression may also be achieved by increasing the copy number of the ORF, for example, by using a higher copy number vector or by using a transposon.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise RUNX1. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise RUNX1. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise RUNX1. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise RUNX1. The one or more CD4-expressing cells may be differentiated from one or more PSCs. The one or more CD4-expressing cells may comprise one or more transcription factors that may induce differentiation of the one or more PSCs into the one or more CD4-expressing cells.

### IPT Domain Containing

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be IPT Domain Containing. The IPT Domain Containing transcription factor may be, for example, NFATC1, NFATC2, or REL.

### NFATC1

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The transcription factor may be NFATC1. NFATC1 may also be known as Nuclear Factor Of Activated T Cells 1, NFATc, NFAT2, NF-ATC, Nuclear Factor Of Activated T-Cells, Cytoplasmic, Calcineurin-Dependent 1, Nuclear Factor Of Activated T-Cells, Cytoplasmic 1, NFAT Transcription Complex Cytosolic Component, Nuclear Factor Of Activated T-Cells ‘C’, Nuclear Factor Of Activated T-Cells 1, NF-ATc1.2, NF-ATc1, NFATc1, NF-ATc, or NFATC.

The present disclosure provides an expression cassette comprising one or more transcription factors. The one or more transcription factors may comprise NFATC1. NFATC1 may be introduced in an expression cassette. NFATC1 may be expressed in an expression cassette. NFATC1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising NFATC1, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise NFATC1. An expression cassette comprising NFATC1 may be introduced in a PSC. An expression cassette comprising NFATC1 may be expressed in a PSC. An expression cassette comprising NFATC1 may induce differentiation of the PSC into a Treg or a FOXP3-expressing cell. Different amounts of NFATC1 may be introduced into the PSC. At least 5, 10, 15, 20, 25, or 50 copies of the open reading frame (ORF) for NFATC1 per cell may be introduced. Increased levels of expression may also be achieved by increasing the copy number of the ORF, for example, by using a higher copy number vector or by using a transposon.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NFATC1. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NFATC1. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NFATC1. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NFATC1. The one or more CD4-expressing cells may be differentiated from one or more PSCs. The one or more CD4-expressing cells may comprise one or more transcription factors that may induce differentiation of the one or more PSCs into the one or more CD4-expressing cells.

### NFATC2

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The transcription factor may be NFATC2. NFATC2 may also be known as Nuclear Factor Of Activated T Cells 2, NFAT1, Nuclear Factor Of Activated T-Cells, Cytoplasmic, Calcineurin-Dependent 2, Nuclear Factor Of Activated T-Cells, Cytoplasmic 2, NFAT Pre-Existing Subunit, NF-ATc2, NF-ATP, NFATp, NFATP, Nuclear Factor Of Activated T-Cells, Preexisting Component, NFAT Transcription Complex, Preexisting Component, Preexisting Nuclear Factor Of Activated T-Cells, Nuclear Factor Of Activated T-Cells 2, T Cell Transcription Factor NFAT1, T-Cell Transcription Factor NFAT1, NFATc2, or NF-ATp.

The present disclosure provides an expression cassette comprising one or more transcription factors. The one or more transcription factors may comprise NFATC2. NFATC2 may be introduced in an expression cassette. NFATC2 may be expressed in an expression cassette. NFATC2 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising NFATC2, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise NFATC2. An expression cassette comprising NFATC2 may be introduced in a PSC. An expression cassette comprising NFATC2 may be expressed in a PSC. An expression cassette comprising NFATC2 may induce differentiation of the PSC into a Treg or a FOXP3-expressing cell. Different amounts of NFATC2 may be introduced into the PSC. At least 5, 10, 15, 20, 25, or 50 copies of the open reading frame (ORF) for NFATC2 per cell may be introduced. Increased levels of expression may also be achieved by increasing the copy number of the ORF, for example, by using a higher copy number vector or by using a transposon.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NFATC2. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NFATC2. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NFATC2. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NFATC2. The one or more CD4-expressing cells may be differentiated from one or more PSCs. The one or more CD4-expressing cells may comprise one or more transcription factors that may induce differentiation of the one or more PSCs into the one or more CD4-expressing cells.

### CUT Class Homeoboxes and Pseudogenes

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be CUT class homeoboxes and pseudogenes. The CUT class homeoboxes and pseudogenes transcription factor may be, for example, SATB1.

### SATB1

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The transcription factor may be SATB1. SATB1 may also be known as transcription factors may be used. A transcription factor may be SATB1. SATB1 may also be known as SATB Homeobox 1, Special AT-Rich Sequence Binding Protein 1 (Binds To Nuclear Matrix/Scaffold-Associating DNA), DNA-Binding Protein SATB1, Special AT-Rich Sequence-Binding Protein 1, DEFDA, or KTZSL.

The present disclosure provides an expression cassette comprising one or more transcription factors. The one or more transcription factors may comprise SATB1. SATB1 may be introduced in an expression cassette. SATB1 may be expressed in an expression cassette. SATB1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising SATB1, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise SATB1. An expression cassette comprising SATB1 may be introduced in a PSC. An expression cassette comprising SATB1 may be expressed in a PSC. An expression cassette comprising SATB1 may induce differentiation of the PSC into a Treg or a FOXP3-expressing cell. Different amounts of SATB1 may be introduced into the PSC. At least 5, 10, 15, 20, 25, or 50 copies of the open reading frame (ORF) for SATB1 per cell may be introduced. Increased levels of expression may also be achieved by increasing the copy number of the ORF, for example, by using a higher copy number vector or by using a transposon.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise SATB1. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise SATB1. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise SATB1. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise SATB1. The one or more CD4-expressing cells may be differentiated from one or more PSCs. The one or more CD4-expressing cells may comprise one or more transcription factors that may induce differentiation of the one or more PSCs into the one or more CD4-expressing cells.

### T-Box Transcription Factors

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be T-box transcription factors. The T-box transcription factor may be, for example, TBX21.

### TBX21

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The transcription factor may be TBX21. TBX21 may also be known as T-Box Transcription Factor 21, TBLYM, T-Bet, T-Cell-Specific T-Box Transcription Factor T-Bet, T-Box Transcription Factor TBX21, Transcription Factor TBLYM, T-Box Protein 21, T-Box 21, TBET, T-Box Expressed In T Cells, IMD88, or T-PET.

The present disclosure provides an expression cassette comprising one or more transcription factors. The one or more transcription factors may comprise TBX21. TBX21 may be introduced in an expression cassette. TBX21 may be expressed in an expression cassette. TBX21 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising TBX21, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise TBX21. An expression cassette comprising TBX21 may be introduced in a PSC. An expression cassette comprising TBX21 may be expressed in a PSC. An expression cassette comprising TBX21 may induce differentiation of the PSC into a Treg or a FOXP3-expressing cell. Different amounts of TBX21 may be introduced into the PSC. At least 5, 10, 15, 20, 25, or 50 copies of the open reading frame (ORF) for TBX21 per cell may be introduced. Increased levels of expression may also be achieved by increasing the copy number of the ORF, for example, by using a higher copy number vector or by using a transposon.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TBX21. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TBX21. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TBX21. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TBX21. The one or more CD4-expressing cells may be differentiated from one or more PSCs. The one or more CD4-expressing cells may comprise one or more transcription factors that may induce differentiation of the one or more PSCs into the one or more CD4-expressing cells.

### MicroRNA Protein Coding Host Genes

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be microRNA protein coding host genes. The microRNA protein coding host gene may be, for example, NFATC2 or NOTCH1.

### NOTCH1

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The transcription factor may be NOTCH1. NOTCH1 may also be known as Notch Receptor 1, Notch 1, TAN1, Translocation-Associated Notch Protein TAN-1, Neurogenic Locus Notch Homolog Protein 1, HN1, Notch (Drosophila) Homolog 1 (Translocation-Associated), Notch Homolog 1, Translocation-Associated (Drosophila), Notch Homolog 1, Translocation-Associated, EC 3.4.21.68, EC 2.1.2.11, AOVD1, or AOS5.

The present disclosure provides an expression cassette comprising one or more transcription factors. The one or more transcription factors may comprise NOTCH1. In some embodiments, transcription factor can be NOTCH1 or a functional derivative thereof. In some embodiments, the functional derivative of NOTCH1 or an intracellular domain of NOTCH1 (also known as NOTCH1 ICD). In some embodiments, NOTCH1 transcription factor herein may comprise NOTCH1 ICD or a functional derivative thereof. In some embodiments, NOTCH1 transcription factor herein may consist of NOTCH1 ICD. NOTCH1 or NOTCH1 ICD or a functional derivatives of NOTCH1 may be introduced in an expression cassette. NOTCH1 may be expressed in an expression cassette. NOTCH1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising NOTCH1, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise NOTCH1. An expression cassette comprising NOTCH1 may be introduced in a PSC. An expression cassette comprising NOTCH1 may be expressed in a PSC. An expression cassette comprising NOTCH1 may induce differentiation of the PSC into a Treg or a FOXP3-expressing cell. Different amounts of NOTCH1 may be introduced into the PSC. At least 5, 10, 15, 20, 25, or 50 copies of the open reading frame (ORF) for NOTCH1 per cell may be introduced. Increased levels of expression may also be achieved by increasing the copy number of the ORF, for example, by using a higher copy number vector or by using a transposon.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NOTCH1. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NOTCH1. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NOTCH1. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise NOTCH1. The one or more CD4-expressing cells may be differentiated from one or more PSCs. The one or more CD4-expressing cells may comprise one or more transcription factors that may induce differentiation of the one or more PSCs into the one or more CD4-expressing cells.

### Notch Receptors

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be notch receptors. The notch receptor may be, for example NOTCH1.

### TCF/LEF Transcription Factor Family

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be TCF/LEF transcription factors. The TCF/LEF transcription factor may be, for example TCF7 or LEF1.

### TCF7

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The transcription factor may be TCF7. TCF7 may also be known as Transcription Factor 7, TCF-1, Transcription Factor 7 (T-Cell Specific, HMG-Box), T-Cell-Specific Transcription Factor 1, T-Cell-Factor-7, T-Cell Factor 1, TCF-7, or TCF1.

The present disclosure provides an expression cassette comprising one or more transcription factors. The one or more transcription factors may comprise TCF7. TCF7 may be introduced in an expression cassette. TCF7 may be expressed in an expression cassette. TCF7 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising TCF7, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise TCF7. An expression cassette comprising TCF7 may be introduced in a PSC. An expression cassette comprising TCF7 may be expressed in a PSC. An expression cassette comprising TCF7 may induce differentiation of the PSC into a Treg or a FOXP3-expressing cell. Different amounts of TCF7 may be introduced into the PSC. At least 5, 10, 15, 20, 25, or 50 copies of the open reading frame (ORF) for TCF7 per cell may be introduced. Increased levels of expression may also be achieved by increasing the copy number of the ORF, for example, by using a higher copy number vector or by using a transposon.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TCF7. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TCF7. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TCF7. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise TCF7. The one or more CD4-expressing cells may be differentiated from one or more PSCs. The one or more CD4-expressing cells may comprise one or more transcription factors that may induce differentiation of the one or more PSCs into the one or more CD4-expressing cells.

### LEF1

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The transcription factor may be LEF1. LEF1 may also be known as Lymphoid Enhancer Binding Factor 1, Lymphoid Enhancer-Binding Factor 1, TCF1ALPHA, TCF7L3, TCF1, T Cell-Specific Transcription Factor 1-Alpha, TCF1-Alpha, or LEF-1.

The present disclosure provides an expression cassette comprising one or more transcription factors. The one or more transcription factors may comprise LEF1. LEF1 may be introduced in an expression cassette. LEF1 may be expressed in an expression cassette. LEF1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising LEF1, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise LEF1. An expression cassette comprising LEF1 may be introduced in a PSC. An expression cassette comprising LEF1 may be expressed in a PSC. An expression cassette comprising LEF1 may induce differentiation of the PSC into a Treg or a FOXP3-expressing cell. Different amounts of LEF1 may be introduced into the PSC. At least 5, 10, 15, 20, 25, or 50 copies of the open reading frame (ORF) for LEF1 per cell may be introduced. Increased levels of expression may also be achieved by increasing the copy number of the ORF, for example, by using a higher copy number vector or by using a transposon.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise LEF1. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise LEF1. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise LEF1. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise LEF1. The one or more CD4-expressing cells may be differentiated from one or more PSCs. The one or more CD4-expressing cells may comprise one or more transcription factors that may induce differentiation of the one or more PSCs into the one or more CD4-expressing cells.

### Wnt Enhanceosome Complex

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be Wnt enhanceosome complex. The Wnt enhanceosome complex transcription factor may be, for example TCF7 or LEFT.

### Nuclear Factors of Activated T-Cells

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be nuclear factors of activated T-cells. The nuclear factor of activated T-cells may be, for example NFATC1 or NFATC2.

### Kruppel Like Factors

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be Kruppel like factors. The Kruppel like factor may be, for example, KLF10.

### Interferon Regulatory Factors

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be interferon regulatory factors. The interferon regulatory factor may be, for example, IRF4.

### Phosphatase 1 Regulatory Subunits

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be phosphatase 1 regulatory subunits. The phosphatase 1 regulatory subunit may be, for example, IKZF1.

### RAR Related Orphan Receptors

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be RAR related orphan receptors. The RAR related orphan receptor may be, for example, RORA.

### RORA

The present disclosure provides immune cells and PSCs comprising a nucleic acid. The nucleic acid may comprise an open reading frame encoding one or more transcription factors, one or more transcription factors, or activator of transcription of the open reading frame encoding one or more transcription factors. The transcription factor may be RORA. RORA may also be known as RAR Related Orphan Receptor A, NR1F1, RZRA, ROR1, ROR2, ROR3, Nuclear Receptor Subfamily 1 Group F Member 1, RAR-Related Orphan Receptor A, Nuclear Receptor ROR-Alpha, Nuclear Receptor RZR-Alpha, Retinoic Acid Receptor-Related Orphan Receptor Alpha, Retinoid-Related Orphan Receptor Alpha, Retinoid-Related Orphan Receptor-Alpha, Transcription Factor RZR-Alpha, RZR-ALPHA, ROR-Alpha, or IDDECA.

The present disclosure provides an expression cassette comprising one or more transcription factors. The one or more transcription factors may comprise RORA. RORA may be introduced in an expression cassette. RORA may be expressed in an expression cassette. RORA may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising RORA, and one or more other transcription factors may induce differentiation of the PSC into a Treg or a FOXP3-expressing cell.

The present disclosure provides a nucleic acid comprising an open reading frame encoding one or more transcription factors, one or more transcription factors, or an activator of transcription of the open reading frame encoding one or more transcription factors, wherein the nucleic acid. The one or more transcription factors may comprise RORA. An expression cassette comprising RORA may be introduced in a PSC. An expression cassette comprising RORA may be expressed in a PSC. An expression cassette comprising RORA may induce differentiation of the PSC into a Treg or a FOXP3-expressing cell. Different amounts of RORA may be introduced into the PSC. At least 5, 10, 15, 20, 25, or 50 copies of the open reading frame (ORF) for RORA per cell may be introduced. Increased levels of expression may also be achieved by increasing the copy number of the ORF, for example, by using a higher copy number vector or by using a transposon.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise RORA. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise RORA. The one or more CD45-expressing cells may be differentiated from one or more PSCs. The one or more CD45-expressing cells may comprise one or more transcription factors that may induce differentiation of the one or more PSCs into the one or more CD45-expressing cells.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise RORA. The one or more CD34-expressing cells may be differentiated from one or more PSCs. The one or more CD34-expressing cells may comprise one or more transcription factors that may induce differentiation of the one or more PSCs into the one or more CD34-expressing cells.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more transcription factors may comprise RORA. The one or more CD4-expressing cells may be differentiated from one or more PSCs. The one or more CD4-expressing cells may comprise one or more transcription factors that may induce differentiation of the one or more PSCs into the one or more CD4-expressing cells.

BAF complex

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be a BAF complex transcription factor. The BAF complex transcription factor may be, for example, IKZF1.

### BTB Domain Containing

The present disclosure provides an immune cell comprising a nucleic acid comprising an open reading frame encoding one or more transcription factors, one or more transcription factors, or an activator of transcription of the open reading frame encoding one or more transcription factors, wherein the nucleic acid. The one or more transcription factors may be a BTB domain containing transcription factor. The BTB domain containing transcription factor may be, for example, ZBTB7B.

### Induced Differentiation

The present disclosure provides methods of differentiating one or more pluripotent stem cells (PSCs). The method may comprise providing a PSC with a nucleic acid comprising an open reading frame encoding one or more transcription factors, one or more transcription factors, or an activator of transcription of the open reading frame encoding one or more transcription factors, wherein the nucleic acid. The PSC may differentiate into an immune cell. The PSC may differentiate into a T cell. The PSC may differentiate into a regulatory T cell. The PSC may differentiate into a CD45-expressing (CD45+) cell. The PSC may differentiate into a CD34-expressing (CD34+) cell. The PSC may differentiate into a CD25-expressing (CD25+) cell. The PSC may differentiate into a CD4-expressing (CD4+) cell. The PSC may be provided in a media. The media may not need alteration during the differentiation of the PSC. The nucleic acid comprising an open reading frame encoding one or more transcription factors, the one or more transcription factors, or the activator of transcription of the open reading frame encoding one or more transcription factors may induce the PSC to differentiate in about 28 days or less. The nucleic acid comprising an open reading frame encoding one or more transcription factors, the one or more transcription factors, or the activator of transcription of the open reading frame encoding one or more transcription factors may induce the PSC to differentiate in about 30 days, about 29 days, about 28 days, about 27 days, about 26 days, about 25 days, about 24 days, about 23 days, about 22 days, about 21 days, about 20 days, about 19 days, about 18 days, about 17 days, about 16 days, about 15 days, about 14 days, about 13 days, about 12 days, about 11 days, about 10 days, about 9 days, about 8 days, about 7 days, about 6 days, about 5 days, about 4 days, about 3 days, about 2 days, about 1 day, or less. The nucleic acid comprising an open reading frame encoding one or more transcription factors, the one or more transcription factors, or the activator of transcription of the open reading frame encoding one or more transcription factors may induce the PSC to differentiate in about 96 hours.

The present disclosure provides one or more Tregs or FOXP3-expressing cells. The one or more Tregs or FOXP3-expressing cells may comprise a nucleic acid. The nucleic acid may comprise an open reading frame encoding one or more transcription factors, one or more transcription factors, or activator of transcription of the open reading frame encoding one or more transcription factors. The one or more Tregs or FOXP3-expressing cells may be differentiated from one or more PSCs. The one or more Tregs or FOXP3-expressing cells may comprise one or more transcription factors that may induce differentiation of the one or more PSCs into the one or more Tregs or FOXP3-expressing cells. At least about 5% of the cells may express FOXP3. At least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, or at least about 40% of the cells may express FOXP3.

The present disclosure provides one or more CD45-expressing cells. The one or more CD45-expressing cells may comprise a nucleic acid. The nucleic acid may comprise an open reading frame encoding one or more transcription factors, one or more transcription factors, or activator of transcription of the open reading frame encoding one or more transcription factors. The one or more CD45-expressing cells may be differentiated from one or more PSCs. The one or more CD45-expressing cells may comprise one or more transcription factors that may induce differentiation of the one or more PSCs into the one or more CD45-expressing cells. The present disclosure provides a population of cells. One or more cells may express CD45. At least about 0.5%, 2%, 3%, 4%, 5%, 6%, 7%, 80%, 9%, 10%, 110%, 12%, 130%, 14%, 150%, 16%, 17%, 18%, 19%, 20%, or more of a population of cells may express CD45. For example, at least about 1% of cells may express CD45. At least about 2% of cells may express CD45. At least about 3% of cells may express CD45. At least about 4% of cells may express CD45. At least about 5% of cells may express CD45. At least about 6% of cells may express CD45. At least about 7% of cells may express CD45. At least about 8% of cells may express CD45. At least about 9% of cells may express CD45. At least about 10% of cells may express CD45. At least about 110% of cells may express CD45. At least about 12% of cells may express CD45. At least about 13% of cells may express CD45. At least about 14% of cells may express CD45. At least about 15% of cells may express CD45. At least about 16% of cells may express CD45. At least about 17% of cells may express CD45. At least about 18% of cells may express CD45. At least about 19% of cells may express CD45. At least about 20% or more of cells may express CD45. In some embodiments, at least about 1% to at least about 6% of a population of cells may express CD45. In some embodiments, at least about 3% to at least about 9% of a population of cells may express CD45.

The present disclosure provides one or more CD34-expressing cells. The one or more CD34-expressing cells may comprise a nucleic acid. The nucleic acid may comprise an open reading frame encoding one or more transcription factors, one or more transcription factors, or activator of transcription of the open reading frame encoding one or more transcription factors. The one or more CD34-expressing cells may be differentiated from one or more PSCs. The one or more CD34-expressing cells may comprise one or more transcription factors that may induce differentiation of the one or more PSCs into the one or more CD34-expressing cells. The present disclosure provides a population of cells. One or more cells may express CD34. At least about 0.5% to at least about 20% of a population of cells may express CD34. At least about 0.5%, 1%, 2%, 3%, 4%, 5%, 6%, 7%, 80%, 9%, 10%, 11%, 12%, 13%, 14%, 150%, 16%, 17%, 18%, 19%, 20%, or more of a population of cells may express CD34. In some embodiments, at least about 2% to at least about 2.5% of a population of cells may express CD34.

The present disclosure provides one or more CD4-expressing cells. The one or more CD4-expressing cells may comprise a nucleic acid. The nucleic acid may comprise an open reading frame encoding one or more transcription factors, one or more transcription factors, or activator of transcription of the open reading frame encoding one or more transcription factors. The one or more CD4-expressing cells may be differentiated from one or more PSCs. The one or more CD4-expressing cells may comprise one or more transcription factors that may induce differentiation of the one or more PSCs into the one or more CD4-expressing cells. The present disclosure provides a population of cells. One or more cells may express CD4. At least about 0.5% to at least about 20% of a population of cells may express CD45. At least about 0.5%, 1%, 2%, 3%, 4%, 5%, 6%, 7%, 8%, 9%, 10%, 11%, 12%, 13%, 14%, 15%, 16%, 17%, 18%, 19%, 20%, or more of a population of cells may express CD4. In some embodiments, at least about 2% to at least about 5% of a population of cells may express CD4.

The present disclosure provides one or more CD8-expressing cells. The one or more CD8-expressing cells may comprise a nucleic acid. The nucleic acid may comprise an open reading frame encoding one or more transcription factors, one or more transcription factors, or activator of transcription of the open reading frame encoding one or more transcription factors. The one or more CD8-expressing cells may be differentiated from one or more PSCs. The one or more CD8-expressing cells may comprise one or more transcription factors that may induce differentiation of the one or more PSCs into the one or more CD8-expressing cells. The present disclosure provides a population of cells. One or more cells may express CD8. At least about 0.5% to at least about 20% of a population of cells may express CD8. At least about 0.5%, 1%, 2%, 3%, 4%, 5%, 6%, 7%, 8%, 9%, 10%, 110%, 12%, 13%, 14%, 15%, 16%, 17%, 18%, 19%, 20%, or more of a population of cells may express CD8. In some embodiments, at least about 2% to at least about 5% of a population of cells may express CD8.

Methods as described herein may be used to generate a population of one or more immune cells. The population of cells may comprise adherent cells. The population of cells may comprise suspension cells. The population of cells may comprise adherent cells and suspension cells. The population of cells may be provided in a media. The media may not have to be altered during the differentiation of the PSCs into immune cells. The media may not need any nutrients, growth factors, or microenvironmental or matrix optimizations.

The present disclosure provides methods for expressing, or for increasing expression of, a transcription factor. A transcription factor may be expressed or have increased expression in a cell through various methods. This may involve delivery of either a nucleic acid comprising an open reading frame encoding the transcription factor, delivery of the transcription factor itself, or delivery of an activator of the transcription factor or its expression. Any technique known in the art, for such delivery may be used. For example, for delivery of a cDNA, a viral or plasmid vector may be used. The open reading frame (encoding any isoform of the TF) may be inducible or repressible for control, to achieve a suitable level of expression. The nucleic acid comprising the open reading frame may be a complementary DNA (cDNA), a messenger RNA (mRNA), or a synthetic or engineered nucleic acid. Some transcription factors may require a critical amount of expression to effectively induce differentiation, such as the equivalent of at least 5, 10, 15, 20, 25, or 50 copies of the ORF per cell. Other factors may require less than a certain threshold of expression due to possible toxicity at high levels, such as less than 20, 10, or 5 copies per cell. Increased levels of expression may also be achieved by increasing the copy number of the ORF, for example, by using a higher copy number vector or by using a transposon. Modified RNAs, RNAs that encode the transcription factor but use synthetic nucleotides that improve stability and reduce degradation, may be used. Use of culture media adapted for a particular cell type may increase the expression of the ORF that induces expression of that cell type. Expression of an ORF can be increased from a non-expressed gene, from a gene expressed at a low level, or from a gene expressed at a robust level. The present disclosure provides methods for expressing, or for increasing expression of, a transcription factor as described in Ng, Alex H M, et al. “A comprehensive library of human transcription factors for cell fate engineering.” Nature biotechnology 39.4 (2021): 510-519; PCT Application Number PCT/US2017/051122; and PCT Application Number PCT/US2018/030216, which are entirely incorporated herein by reference.

It will be understood that methods for increasing the expression of the transcription factors in the cells to be programmed into immune cells may include any method known in the art, for example, by induction of expression of one or more expression cassettes previously introduced into the cells, or by introduction of nucleic acids (such as DNA or RNA), polypeptides, or small molecules to the cells. Increasing the expression of certain endogenous but transcriptionally repressed genes may also reverse the silencing or inhibitory effect on the expression of these genes by regulating the upstream transcription factor expression or epigenetic modulation. Therefore, methods of the invention may involve culturing the cell population under conditions to artificially increase the expression level of one or more of the transcription factors described herein.

Contact with Transcription Factors

The present disclosure provides methods for expressing, or for increasing expression of, a transcription factor. The expression of the transcription factors may be increased by contacting at least one cell with one or more proteins encoding transcription factors. The at least one cell may be a stem cell. The stem cell may be a pluripotent stem cell. The pluripotent stem cell may be an induced pluripotent stem cell (iPSC). Delivery of the transcription factors may occur using direct electroporation of transcription factor proteins to the cells.

Direct Contact with Agents

The present disclosure provides methods for expressing, or for increasing expression of, a transcription factor. The expression of the transcription factors may be increased by contacting at least one cell with one or more agents that activate or increase the expression or amount of the transcription factors. The at least one cell may be a stem cell. The stem cell may be a pluripotent stem cell. The pluripotent stem cell may be an induced pluripotent stem cell (iPSC). Delivery of the transcription factors may occur using direct electroporation of transcription factor proteins to the cells. The agent may be a nucleic acid (i.e., polynucleotide, e.g., messenger RNA (mRNA), coding DNA sequence), a protein, an aptamer and small molecule, ribosome, RNAi agent, guide RNA (gRNA) and peptide-nucleic acid (PNA) and analogues or variants thereof. In one embodiment, the agent is a transcriptional activation system (e.g., a gRNA for use in a gene activation system such as CRISPR/Cas9 or TALEN) for increasing the expression of the one or more endogenous transcription factors.

### Open Reading Frame

The present disclosure provides methods for expressing, or for increasing expression of, a transcription factor. The expression of the transcription factors may be increased by inducing differentiation of at least one cell by delivering to the at least one cell a nucleic acid comprising an open reading frame encoding one or more of the transcription factors, the transcription factor protein, or an activator of transcription of the open reading frame encoding one or more transcription factors. The one or more transcription factors may be delivered in an exogenous expression cassette. The at least one cell may be a stem cell. The stem cell may be a pluripotent stem cell. The pluripotent stem cell may be an induced pluripotent stem cell (iPSC).

### Exogenous Expression Cassette

The present disclosure provides methods for expressing, or for increasing expression of, a transcription factor. The expression of the transcription factors may be increased by delivering one or more nucleic acids encoding a transcription factor or an activator of transcription of the open reading frame encoding one or more transcription factors. The nucleic acid may comprise a recombinant or exogenous expression cassette encoding one or more transcription factors. The recombinant or exogenous expression cassette may induce the cell to differentiate into a hematopoietic lineage. The recombinant or exogenous expression cassette may induce the cell to differentiate into an immune cell for example, a regulator T cell. The at least one cell may be a stem cell. The stem cell may be a pluripotent stem cell. The pluripotent stem cell may be an induced pluripotent stem cell (iPSC). One or more recombinant or exogenous expression cassettes may be used. The exogenous expression cassette may comprise an externally inducible transcriptional regulatory element for inducible expression of the one or more transcription factors, such as an inducible promoter, e.g., comprising a tetracycline response element or variant thereof. Any suitable system for delivering the sequence may be used. The gene delivery system may be a transposon system or a viral gene delivery system or an episomal gene delivery system or a homologous recombination system, such as, for example, utilizing a zinc finger nuclease, a transcription activator-like effector nuclease (TALENs), or a meganuclease, or a CRISPR/Cas9, or the like.

The exogenous expression cassette may include cleavable sequences. Such cleavable sequences are sequences that are recognized by an entity capable of specifically cutting DNA, and include restriction sites, which are the target sequences for restriction enzymes or sequences for recognition by other DNA cleaving entities, such as nucleases, recombinases, ribozymes, or artificial constructs. At least one cleavable sequence may be included, but preferably two or more may be present. These cleavable sequences may be at any suitable point in the cassette, such that a selected portion of the cassette, or the entire cassette, can be selectively removed if desired. The cleavable sites may thus flank the part or may flank all of the genetic sequence that it may be desired to remove. The method may therefore also comprise removal of the expression cassette and/or the genetic material.

### Direct Delivery of DNA

The present disclosure provides methods for expressing, or for increasing expression of, a gene product, such as a transcription factor. The expression of the transcription factors may be increased by directly delivering one or more nucleic acids to a cell. The at least one cell may be a stem cell. The stem cell may be a pluripotent stem cell. The pluripotent stem cell may be an induced pluripotent stem cell (iPSC). The nucleic acid may be a DNA or RNA. The one or more nucleic acids may be delivered into cells using any suitable methods for nucleic acid delivery for transformation of a cell known in the art. Such methods include, but are not limited to, direct delivery of DNA such as by ex vivo transfection, by injection (including microinjection), by electroporation, by calcium phosphate precipitation, by using DEAE-dextran followed by polyethylene glycol, by direct sonic loading, by liposome mediated transfection, by receptor-mediated transfection, by microprojectile bombardment, by agitation with silicon carbide fibers, by Agrobacterium-mediated transformation, and any combination of such methods.

### Vectors

The present disclosure provides methods for expressing, or for increasing expression of a gene product, such as for example, a transcription factor. The expression of the transcription factors may be increased by delivering into a cell a nucleic acid comprising an open reading frame encoding one or more of the transcription factors, the transcription factor protein, or an activator of transcription of the open reading frame encoding one or more transcription factors to at least one cell. The at least one cell may be a stem cell. The stem cell may be a pluripotent stem cell. The pluripotent stem cell may be an induced pluripotent stem cell (iPSC). The nucleic acids may be introduced into the cell using a vector. A vector can be constructed through any standard techniques known in the art. Vectors include but are not limited to plasmids, cosmids, viruses (bacteriophage, animal viruses, and plant viruses), and artificial chromosomes (e.g., YACs) among other vectors known in molecular cloning techniques.

The vector may be a viral vector. The viral gene delivery system may be an RNA-based or DNA-based viral vector. For example, the viral vectors include retroviral vectors, lentiviral vectors (e.g., derived from HIV-1, HIV-2, SIV, BIV, FIV etc.), gammaretroviral vectors, adenoviral (Ad) vectors (including replication competent, replication deficient and gutless forms thereof), adeno-associated virus-derived (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors and Sendai virus vectors. The viral vector may be selected from a lentiviral vector, an adeno-associated virus vector or a Sendai virus vector. In a yet further embodiment, the viral vector is a lentiviral vector. In one embodiment, the viral vector is used at a high multiplicity of infection (MOI). A high MOI helps to ensure that more than one transcription factor is introduced into the source cell. In one embodiment, the MOI is greater than 0.5, such as 1.0 or above.

### Plasmids

The present disclosure provides methods for expressing, or for increasing expression of, a transcription factor. The expression of the transcription factors may be increased by delivering cell a nucleic acid comprising an open reading frame encoding one or more of the transcription factors, the transcription factor protein, or an activator of transcription of the open reading frame encoding one or more transcription factors to at least one cell. The at least one cell may be a stem cell. The stem cell may be a pluripotent stem cell. The pluripotent stem cell may be an induced pluripotent stem cell (iPSC). The nucleic acids may be introduced into the cell using a plasmid.

The plasmid may be episomal. Episomal vectors are able to introduce large fragments of DNA into a cell but are maintained extra-chromosomally, replicated once per cell cycle, partitioned to daughter cells efficiently, and elicit substantially no immune response. In alternative embodiments, an Epstein-Barr virus (EBV)-based episomal vector, a yeast-based vector, an adenovirus-based vector, a simian virus 40 (SV40)-based episomal vector, or a bovine papilloma virus (BPV)-based vector may be used.

### Site-Specific Delivery

Any suitable technique for insertion of a nucleic acid sequence into a specific sequence may be used, and several are described in the art. Suitable techniques may include any method which introduces a break at the desired location and permits recombination of the vector into the gap. Thus, a crucial first step for targeted site-specific genomic modification is the creation of a double-strand DNA break (DSB) at the genomic locus to be modified. Distinct cellular repair mechanisms can be exploited to repair the DSB and to introduce the desired sequence, and these are non-homologous end joining repair (NHEJ), which is more prone to error; and homologous recombination repair (HR) mediated by a donor DNA template, that can be used to insert inducible cassettes.

Several techniques exist to allow customized site-specific generation of DSB in the genome. Many of these involve the use of customized endonucleases, such as zinc finger nucleases, TALENs or the clustered regularly interspaced short palindromic repeats/CRISPR associated protein (CRISPR/Cas9) system.

Zinc finger nucleases are artificial enzymes which are generated by fusion of a zinc-finger DNA-binding domain to the nuclease domain of the restriction enzyme FokI. The latter has a non-specific cleavage domain which must dimerize in order to cleave DNA. This means that two zinc finger nuclease monomers are required to allow dimerization of the FokI domains and to cleave the DNA. The DNA binding domain may be designed to target any genomic sequence of interest, is a tandem array of Cys2His2 zinc fingers, each of which recognizes three contiguous nucleotides in the target sequence. The two binding sites are separated by 5-7 bp to allow optimal dimerization of the FokI domains. The enzyme thus is able to cleave DNA at a specific site, and target specificity is increased by ensuring that two proximal DNA-binding events must occur to achieve a double-strand break.

Transcription activator-like effector nucleases, or TALENs, are dimeric transcription factor/nucleases. They are made by fusing a TAL effector DNA-binding domain to a DNA cleavage domain (a nuclease). Transcription activator-like effectors (TALEs) can be engineered to bind practically any desired DNA sequence, so when combined with a nuclease, DNA can be cut at specific locations. TAL effectors are proteins that are secreted by Xanthomonas bacteria, the DNA binding domain of which contains a repeated highly conserved 33-34 amino acid sequence with divergent 12th and 13th amino acids. These two positions are highly variable and show a strong correlation with specific nucleotide recognition. This straightforward relationship between amino acid sequence and DNA recognition has allowed for the engineering of specific DNA-binding domains by selecting a combination of repeat segments containing appropriate residues at the two variable positions. TALENs are thus built from arrays of 33 to 35 amino acid modules, each of which targets a single nucleotide. By selecting the array of the modules, almost any sequence may be targeted. Again, the nuclease used may be FokI or a derivative thereof.

Three types of CRISPR mechanisms have been identified, of which type II is the most studied. The CRISPR/Cas9 system (type II) utilizes the Cas9 nuclease to make a double-stranded break in DNA at a site determined by a short guide RNA. The CRISPR/Cas system is a prokaryotic immune system that confers resistance to foreign genetic elements. CRISPR are segments of prokaryotic DNA containing short repetitions of base sequences. Each repetition is followed by short segments of “protospacer DNA” from previous exposures to foreign genetic elements. CRISPR spacers recognize and cut the exogenous genetic elements using RNA interference. The CRISPR immune response occurs through two steps: CRISPR-RNA (crRNA) biogenesis and crRNA-guided interference. CrRNA molecules are composed of a variable sequence transcribed from the protospacer DNA and a CRISPR repeat. Each crRNA molecule then hybridizes with a second RNA, known as the trans-activating CRISPR RNA (tracrRNA) and together these two eventually form a complex with the nuclease Cas9. The protospacer DNA encoded section of the crRNA directs Cas9 to cleave complementary target DNA sequences, if they are adjacent to short sequences known as protospacer adjacent motifs (PAMs). This natural system has been engineered and exploited to introduce DSB breaks in specific sites in genomic DNA, amongst many other applications. In particular, the CRISPR type II system from Streptococcus pyogenes may be used. At its simplest, the CRISPR/Cas9 system comprises two components that are delivered to the cell to provide genome editing: the Cas9 nuclease itself and a gRNA. The gRNA is a fusion of a customized, site-specific crRNA (directed to the target sequence) and a standardized tracrRNA.

Once a DSB has been made, a donor template with homology to the targeted locus is supplied; the DSB may be repaired by the homology-directed repair (HDR) pathway allowing for precise insertions to be made.

Derivatives of this system are also possible. Mutant forms of Cas9 are available, such as Cas9D10A, with only nickase activity. This means it cleaves only one DNA strand and does not activate NHEJ. Instead, when provided with a homologous repair template, DNA repairs are conducted via the high-fidelity HDR pathway only. Cas9D10A may be used in paired Cas9 complexes designed to generate adjacent DNA nicks in conjunction with two sgRNAs complementary to the adjacent area on opposite strands of the target site, which may be particularly advantageous.

The elements for making the double-strand DNA break may be introduced in one or more vectors, such as plasmids, for expression in the cell.

Thus, any method of making specific, targeted double strand breaks in the genome in order to insert a gene/inducible cassette may be used in the method of the invention. It may be preferred that the method for inserting the gene/inducible cassette utilizes any one or more of zinc finger nucleases, TALENs and/or CRISPR/Cas9 systems or any derivative thereof.

Once the DSB has been made by any appropriate means, the gene/inducible cassette for insertion may be supplied in any suitable fashion as described below. The gene/inducible cassette and associated genetic material form the donor DNA for repair of the DNA at the DSB and are inserted using standard cellular repair machinery/pathways. How the break is initiated will alter which pathway is used to repair the damage, as noted above.

### Controlled Expression

In one embodiment, expression of the transcription factors is under controlled transcription. In this aspect of the invention, the transcription and translation (expression) of the transcription factors may be controlled within the cell. This permits overexpression of the transcription factor(s), if required.

An exogenous expression cassette carrying the transcription factors may comprise an externally inducible transcriptional regulatory element (i.e., an inducible promoter) for inducible expression of the transcription factors. The inducible expression cassette may be controlled by addition of an exogenous substance. Whatever culturing conditions are used, the exogenous substance will control expression of the genetic sequence within the inducible expression cassette; and may either be supplied continuously and then withdrawn in order to induce transcription or supplied as transcription is required, dependent upon its mode of action.

Expression of the transcription factors described herein may be increased using a dual cassette expression system. This system targets genetic safe harbor (GSH) sites which provides a reduced risk of epigenetic silencing of the inserted genetic material.

A GSH site is a locus within the genome wherein a gene or other genetic material may be inserted without any deleterious effects on the cell or on the inserted genetic material. Most beneficial is a GSH site in which expression of the inserted gene sequence is not perturbed by any read-through expression from neighbouring genes and expression of the inducible cassette minimizes interference with the endogenous transcription activity. More formal criteria have been proposed that assist in the determination of whether a particular locus is a GSH site in future (Papapetrou et al., (2011)) These criteria include a site that is (i) 50 kb or more from the 5′ end of any gene, (ii) 300 kb or more from any gene related to cancer, (iii) 300 kb or more from any microRNA (miRNA), (iv) located outside a transcription unit and (v) located outside ultra-conserved regions (UCR). It may not be necessary to satisfy all of these proposed criteria, since GSH already identified do not fulfil all of the criteria. It is thought that a suitable GSH will satisfy at least 2, 3, 4 or all of these criteria. Any suitable GSH site may be used in the method of the invention, on the basis that the site allows insertion of genetic material without deleterious effects to the cell and permits transcription of the inserted genetic material. Those skilled in the art may use these simplified criteria to identify a suitable GSH, and/or the more formal criteria set out above.

Specific insertion of genetic material into the particular GSH based upon customized site-specific generation of DNA double-strand breaks at the GSH may be achieved. The genetic material may then be introduced using any suitable mechanism, such as homologous recombination. Any method of making a specific DSB in the genome may be used, but preferred systems include CRISPR/Cas9 and modified versions thereof, zinc finger nucleases and the TALEN system.

One or more genetic sequences may be controllably transcribed from within the second and/or further GSH. Indeed, the inducible cassette may contain 1, 2, 3, 4, 5, 6, 7, 8, 9 or 10 genetic sequences (e.g., transcription factor sequences) which it is desired to insert into the GSH and the transcription of which be controllably induced. Therefore, the transcription factors required by the present invention may be included within the same cassette introduced into the second genetic safe harbor site. For example, the three or more transcription factors may be included in, for example, three mono-cistronic constructs, one mono-cistronic and one bi-cistronic construct or one tri-cistronic construct. It will be understood that similar combinations of constructs may be used to achieve higher orders of transcription factor expression.

Alternatively, if a combination of transcription factors is used, the individual transcription factors may be introduced into separate GSHs and/or under the control of different inducible promoters. Therefore, in one embodiment, the at least three or more transcription factors are introduced into separate GSHs. This may be achieved by utilizing three or more different GSH sites for the three or more transcription factors (i.e., wherein the transcription factors are introduced as mono-cistronic cassettes). Alternatively, this may be achieved by utilizing the fact that a GSH exists at the same genetic loci on both chromosomes of diploid organisms, e.g., introducing one transcription factor into the GSH on one chromosome and a different transcription factor into the same GSH on the other chromosome. This embodiment is advantageous if different expression levels or timing of expression of the transcription factors is desired. In one embodiment, the method comprises targeted insertion of the at least three or more transcription factors, each operably linked to an inducible promoter into a second, third and fourth genetic safe harbor site of the source cell. The inducible promoter may be the same of each transcription factor and therefore are all regulated by the transcriptional regulator protein.

A transcriptional regulator protein is a protein that binds to DNA, preferably sequence-specifically to a DNA site located in or near a promoter, and either facilitating the binding of the transcription machinery to the promoter, and thus transcription of the DNA sequence (a transcriptional activator) or blocks this process (a transcriptional repressor).

The DNA sequence that a transcriptional regulator protein binds to is called a transcription factor-binding site or response element, and these are found in or near the promoter of the regulated DNA sequence. Transcriptional activator proteins bind to the response element and promote gene expression. Such proteins are preferred in the methods of the present invention for controlling inducible cassette expression. Transcriptional repressor proteins bind to the response element and prevent gene expression.

Transcriptional regulator proteins may be activated or deactivated by a number of mechanisms including binding of a substance, interaction with other transcription factors (e.g., homo- or hetero-dimerization) or coregulatory proteins, phosphorylation, and/or methylation. The transcriptional regulator protein may be controlled by activation or deactivation. Any suitable transcriptional regulator protein may be used, preferably one that may be activated or deactivated. It is preferred that an exogenous substance may be supplied to control the transcriptional regulator protein. Such transcriptional regulator proteins are also called inducible transcriptional regulator proteins.

### Tetracycline-Controlled Transcriptional Activation

Tetracycline-Controlled Transcriptional Activation is a method of inducible gene expression where transcription is reversibly turned on or off in the presence of the antibiotic tetracycline or one of its derivatives (e.g., doxycycline which is more stable). In this system, the transcriptional activator protein is tetracycline-responsive transcriptional activator protein (rtTa) or a derivative thereof. The rtTA protein is able to bind to DNA at specific TetO operator sequences. Several repeats of such TetO sequences are placed upstream of a minimal promoter (such as the CMV promoter), which together form a tetracycline response element (TRE). There are two forms of this system, depending on whether the addition of tetracycline or a derivative activates (Tet-On) or deactivates (Tet-Off) the rtTA protein.

In a Tet-Off system, tetracycline or a derivative thereof binds rtTA and deactivates the rtTA, rendering it incapable of binding to TRE sequences, thereby preventing transcription of TRE-controlled genes. This system was first described in Gossen et al., (1992).

The Tet-On system is composed of two components; (1) the constitutively expressed tetracycline-responsive transcriptional activator protein (rtTa) and the rtTa-sensitive inducible promoter (Tet Responsive Element, TRE). This may be bound by tetracycline or its more stable derivatives, including doxycycline (dox), resulting in activation of rtTa, allowing it to bind to TRE sequences and inducing expression of TRE-controlled genes. The use of this may be preferred in the method of the invention.

Thus, the transcriptional regulator protein may thus be tetracycline-responsive transcriptional activator protein (rtTa) protein, which can be activated or deactivated by the antibiotic tetracycline or one of its derivatives, which are supplied exogenously. If the transcriptional regulator protein is rtTA, then the inducible promoter inserted into the second GSH site includes the tetracycline response element (TRE). The exogenously supplied substance is the antibiotic tetracycline or one of its derivatives. Variants and modified rtTa proteins may also be used in the methods of the invention, these include Tet-On Advanced transactivator (also known as rtTA2S-M2) and Tet-On 3G (also known as rtTA-V16, derived from rtTA2S-52).

The tetracycline response element (TRE) generally consists of 7 repeats of the 19 bp bacterial TetO sequence separated by spacer sequences, together with a minimal promoter. Variants and modifications of the TRE sequence are possible since the minimal promoter can be any suitable promoter. Preferably the minimal promoter shows no or minimal expression levels in the absence of rtTa binding. The inducible promoter inserted into the second GSH may thus comprise a TRE.

A modified system based upon tetracycline control is the T-REX System (Thermo-Fisher Scientific), in which the transcriptional regulator protein is a transcriptional repressor protein, TetR. The components of this system include (i) an inducible promoter comprising a strong human cytomegalovirus immediate-early (CMV) promoter and two tetracycline operator 2 (TetO2) sites, and a Tet repressor (TetR). In the absence of tetracycline, the Tet repressor forms a homodimer that binds with extremely high affinity to each TetO2 sequence in the inducible promoter and prevent transcription from the promoter. Once added, tetracycline binds with high affinity to each Tet repressor homodimer rendering it unable to bind to the Tet operator. The Tet repressor: tetracycline complex then dissociates from the Tet operator and allows induction of expression. In this instance, the transcriptional regulator protein is TetR, and the inducible promoter comprises two TetO2 sites. The exogenously supplied substance is tetracycline or a derivative thereof. Other inducible expression systems are known and can be used in the method of the invention. These include the Complete Control Inducible system from Agilent Technologies. This is based upon the insect hormone ecdysone or its analogue ponasterone A (ponA) which can activate transcription in mammalian cells which are transfected with both the gene for the Drosophila melanogaster ecdysone receptor (EcR) and an inducible promoter comprising a binding site for the ecdysone receptor. The EcR is a member of the retinoid-X-receptor (RXR) family of nuclear receptors. In humans, EcR forms a heterodimer with RXR that binds to the ecdysone-responsive element (EcRE). In the absence of PonA, transcription is repressed by the heterodimer.

### Applications

The differentiated cells that can be produced using the methods described in the present disclosure may have various applications. They can be used for regenerative medicine, such as transplanting the cells into a recipient in need of a certain type of cell. They can be used for drug testing, both in cell culture as well as after transplantation. The cells may be used to deliver a product to a part of a body, for example, if they naturally produce or are engineered to produce and secrete the product.

Drug testing in the cells may use substances that are known or unknown to have a certain biological activity. The substances may be elements, compounds, or mixtures, whether natural or synthetic. The cells may be used to determine a desirable activity of a potential drug or conversely to determine undesirable effects of a substance or lack of such effects. The contacting of the substance with the cells may be in culture or in a human or animal body. The activity or side effects of the substance may be determined in vitro or in vivo, irrespective of where the contacting occurred.

The cells can be observed for effects on cell growth, differentiation, apoptosis, secreted products, expression of particular products, etc. The genome of these cells may be edited to match mutations found in patients with disease. Any type of assay known in the art for such changes may be used, including but not limited to immunological assays, morphological observations, histochemical stains, reverse transcription polymerase chain reaction, protein blots, mass spectrometry, hybridization assays, electrophysiology, etc.

Stem cells of the present disclosure may be obtained from any source. One useful source may be human induced pluripotent stem cells. Mouse induced pluripotent stem cells and mouse embryonic stem cells may also be used, as well as such cells from other animals. The use of human embryonic stem cells may be regulated or ethically undesirable, but these may be used as well. Differentiated cells may be identified by any property or set of properties that is characteristic or defining of that type of differentiated cell. For example, different cell types have a unique transcriptome. The transcriptome may be used as a means of matching and identifying an unknown cell type to a known cell type. The transcriptome may be used qualitatively or quantitatively. Similarly, a proteome may be used a means of identifying an unknown differentiated cell type. Some cell types may be identifiable based on morphology, growth habit, secretion products, enzymatic activity, cellular function, and the like. Any means known in the art for identifying cells may be used.

### Definitions

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

“Overexpression” is expression at a level that is higher than the level that is expressed before induction from a gene that is expressed at a low, medium, or high level. An exogenous open reading frame is typically an open reading frame that differs from the similar gene or mRNA in the cell. It may be engineered to have a different control sequence or sequences, such as promoter, operator, enhancer, terminator, etc. It may be engineered to have no introns. It may be engineered to be fused to a second open reading frame to which it is not fused in the human genome.

As used herein, an “immune cell” refers to a cell associated with the immune system that is at any stage of development. Examples of an immune cell may include a hematopoietic progenitor cell or a mature T cell. Examples of a T cell may include a regulatory T cell. The immune cells produced by methods as described herein may be at least as functional as the immune cells produced by directed differentiation to date. Immune cells may express FOXP3. Immune cells may express CD45. Immune cells may express CD34. Immune cells may express CD4.

The above disclosure generally describes the present invention. All references disclosed herein are expressly incorporated by reference. A more complete understanding can be obtained by reference to the following specific example, which are provided herein for purposes of illustration only, and is not intended to limit the scope of the invention.

### EXAMPLES

### Example 1: TF Recipe Induced Expression of Endogenous FOXP3

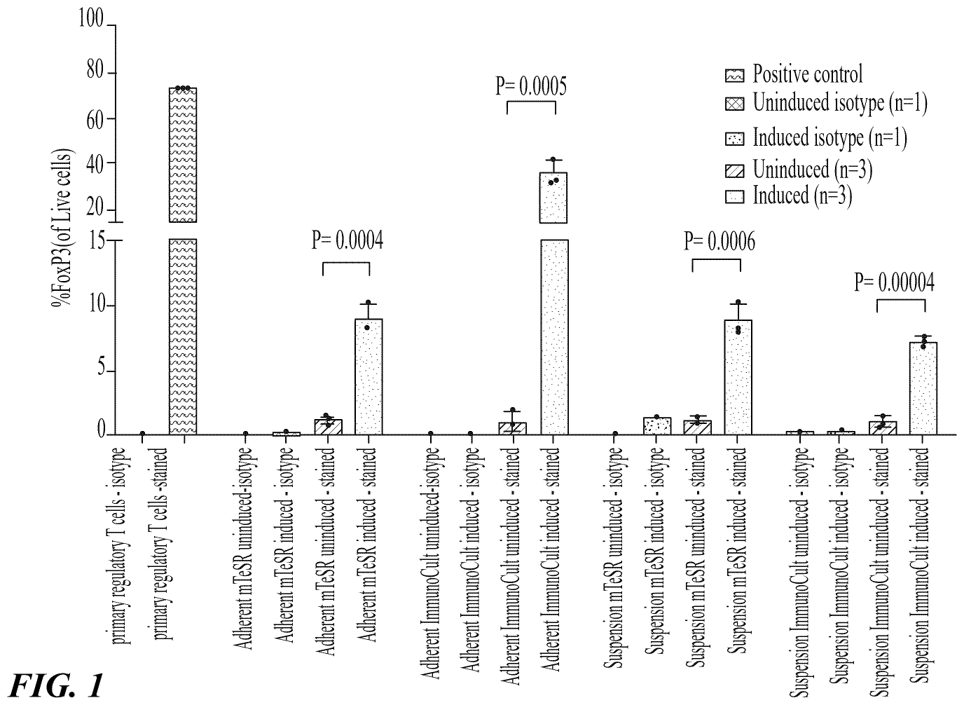

Flow cytometry was used to assess the expression of FOXP3. pcRTang which are primary regulatory T cells, showed no signal when a negative control isotype stain was used. When pcRTang were stained with anti-FOXP3 antibodies as a positive control, about 70% of cells expressed FOXP3 (FIG. 1). 5C3 cells were cultivated in adherent cell culture with induction of one or more transcription factors (TFs)(example of transcription factors, e.g., BCL11B, RUNX1, HEB, ZBTB7B, GABPA, GATA3, or NFKB1) or without induction of any TF in mTeSR1 stem cell media. A negative control isotype antibody indicated no expression was detected. When 5C3 cells were grown in adherent cell culture without TF induction and stained with anti-FOXP3 antibodies, minimal FOXP3 was detected (about 1% of cells). However, when 5C3 cells were cultivated with TF induction for 96 hours and stained with anti-FOXP3 antibodies, about 8% of the cells expressed FOXP3. This was repeated experiment using ImmunoCult culturing media, which is used to grow immune cells, and a boost in FOXP3 expression was observed in which about 40% of TF-induced cells expressed FOXP3. Similar results were obtained when cells were grown in suspension conditions: in both mTeSR stem cell media and ImmunoCulture media, about 8% of cells expressed FOXP3. Overall, these data indicate that the TF recipe or TF cocktail employed induced endogenous FOXP3 expression robustly, indicating 8% of the cells differentiated to regulatory T cells (immune cell lineage); TF induction was observed in all four conditions, namely, when cells were cultivated in mTeSR stem cell media or in ImmunoCult media, and when cells cultivated as adherent cells or as suspension cells (FIG. 1).

### Example 2: Markers of Hematopoietic or Immune T Cell Lineages Identified

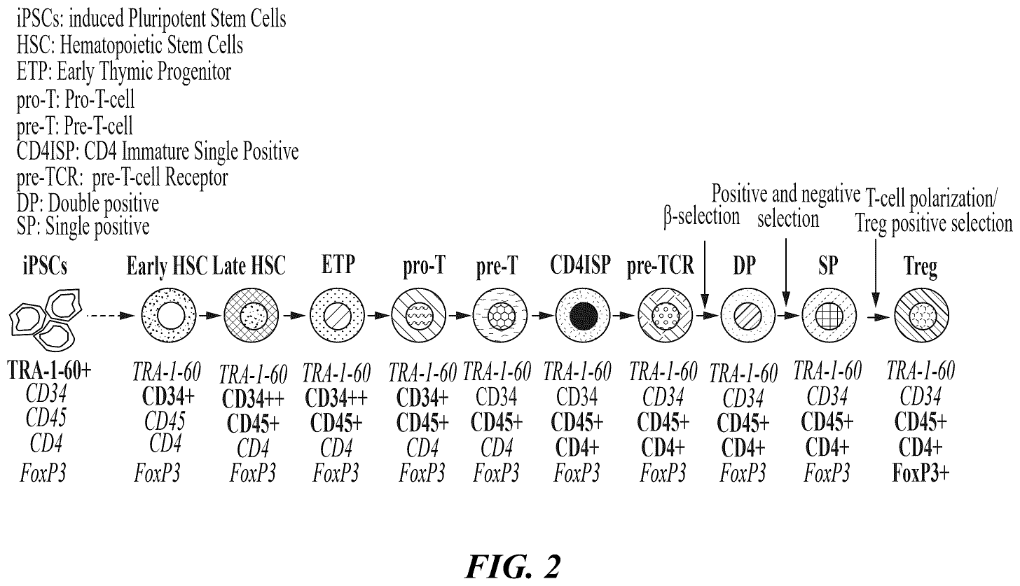

Genes were screened and assessed for hematopoietic/immune cell lineages markers, including the earlier stage hematopoietic progenitors to the T cell stage (FIG. 2). Two markers of hematopoietic progenitors are the proteins CD34 and CD45. CD34 is a cell surface marker that aids in the attachment of hematopoietic cells in the bone marrow and is used to identify early hematopoietic lineages. As indicated in FIG. 2, several other gene products can be used as markers to identify cell differentiation or cell lineages, for example and without any limitation, FoxP3 is a specific marker of regulator T cells (Tregs). CD45 is a protein tyrosine phosphatase that is expressed in all leukocytes and is a marker of committed progenitors and is expressed throughout the progenitor stage and in mature T cells (FIG. 2). Cells were tested for expression of the T cell lineage marker, CD4, which is a co-receptor for the T Cell Receptor (TCR) and plays a role in the recognition of antigen presenting cell binding to the Major Histocompatibility Complex II (MHC class II) protein complex.

Each gene was cloned into an expression vector (plasmid), then transfected into iPSCs. The recombinant expression plasmid was designed to carry an antibiotic resistant gene and bear small molecule-inducible capability in order to allow for the selection of TF-integrated cells and to express the TFs on demand, respectively.

It was successfully shown that different gene markers from the hematopoietic/T cell lineages can be detected after inducing expression of different TF recipes, or combinations, some of which are depicted in Table 1-Table 3. The TF recipes or cocktails depicted in the present disclosure are associated with various TF families, some of which are disclosed in Table 4.

TABLE 1
Recipes, cocktails, or combinations of transcription factors

| #4 | #6 | #11 | #13 | #17 | #19 | #20 | #21 |
|---|---|---|---|---|---|---|---|
| IKZF4 | BCL11B | FOXO1 | BCL11B | BCL11B | FOXO1 | FOXP3 | DXT1 |
| DTX1 | DTX1 | GATA1 | DTX1 | DTX1 | FOXO3 | GATA1 | FOXO1 |
| FOXO1 | FOXO3 | GATA3 | FOXO1 | FOXP3 | FOXP3 | GATA3 | FOXO3 |
| NOTCH1 | GATA1 | IKZF1 | GATA3 | GATA3 | GATA3 | IKFZ1 | FOXP3 |
| GATA1 | GATA3 | IKZF2 | KLF10 | IKZF2 | IKZF4 | IKFZ4 | IKZF1 |
| NFATC1 | IKZF1 | IRF4 | NFATC1 | IKZF4 | KLF10 | NFATC1 | IRF4 |
| IKZF2 | LEF1 | KLF10 | NFATC2 | IRF4 | LEF1 | NFATC2 | NFATC1 |
| LEF1 | NOTCH1 | NOTCH1 | NOTCH1 | NOTCH1 | NOTCH1 | NOTCH1 | NOTCH1 |
| REL | RORA | REL | REL | RUNX1 | RORA | RUNX1 | RORA |
| RUNX1 | SATB1 | TCF12 | SATB1 | SATB1 | REL | TCF12 | REL |
| SATB1 | TBX21 | TCF3 | TBX21 | TBX21 | RUNX1 | TCF7 | SATB1 |
| ZBTB7B | TCF12 | ZBTB7B | TCF12 | TCF12 | TCF12 | ZBTB7B | TCF3 |
| | ZBTB7B | | TCF7 | TCF3 | | | |

TABLE 2
Additional recipes, cocktails, or combinations of transcription factors

| #23 | #28 | #32 | #35 | #2 | #3 | #10 |
|---|---|---|---|---|---|---|
| BCL11B | DTX1 | BCL11B | FOXO3 | FOXO1 | BCL11B | DTX1 |
| FOXO1 | FOXP3 | FOXP3 | GATA3 | FOXO3 | FOXO3 | FOXO1 |
| FOXO3 | GATA1 | IKZF1 | IKZF2 | GATA1 | FOXP3 | FOXP3 |
| FOXP3 | IRF4 | IKZF2 | IRF4 | IKZF1 | GATA3 | GATA3 |
| GATA1 | KLF10 | KLF10 | LEF1 | KLF10 | IKZF1 | IKZF1 |
| IKZF2 | LEF1 | LEF1 | NFATC1 | LEF1 | IRF4 | IKZF2 |
| NFATC2 | NFATC2 | NFATC1 | NFATC2 | NFATC1 | KLF10 | LEF1 |
| NOTCH1 | NOTCH1 | NOTCH1 | NOTCH1 | NFATC2 | NFATC2 | NFATC2 |
| RORA | SATB1 | TBX21 | RORA | RUNX1 | REL | RORA |
| REL | TCF3 | | TCF12 | SATB1 | RUNX1 | RUNX1 |
| TBX21 | TCF7 | | TCF3 | TBX21 | SATB1 | TBX21 |
| TCF7 | ZBTB7B | | TCF7 | TCF12 | ZBTB7B | TCF3 |
| ZBTB7B | | | | TCF3 | | ZBTB7B |

TABLE 3
Additional recipes, cocktails, or combinations of transcription factors
CD34 recipe

| | | | | |
|---|---|---|---|---|
| DTX1 | GATA3 | IKZF4 | NFATC1 | TCF3 |
| BCL11B | GATA1 | IRF4 | TBX21 | TCF7 |
| FOXO1 | IKZF1 | NOTCH1 | NFATC2 | TCF12 |
| FOXO3 | IKZF2 | REL | LEF1 | SATB1 |
| FOXP3 | KLF10 | RORA | RUNX1 | ZBTB7B |

The gene families of each TF are also included in the second column of Table 4.

TABLE 4
Gene families associated with transcription factors

| | TFs Gene Families |
|---|---|
| BCL11B | Zinc fingers C2H2-type, BAF |
| | complex |
| DTX1 | Ring finger proteins |
| FOXO1 | Forkhead boxes |
| FOXO3 | Forkhead boxes |
| FOXP3 | Forkhead boxes |
| GATA1 | GATA zinc finger domain |
| | containing |
| GATA3 | GATA zinc finger domain |
| | containing |
| IKZF1 | Zinc fingers C2H2-type |
| | Protein, phosphatase 1 |
| | regulatory subunits |
| IKZF2 | Zinc fingers C2H2-type |
| IKZF4 | Zinc fingers C2H2-type |
| IRF4 | Interferon regulatory factors |
| KLF10 | Zinc fingers C2H2-type, |
| | Kruppel like factors |
| LEF1 | TCF/LEF transcription factor |
| | family, Wnt enhanceosome |
| | complex |
| NFATC1 | Nuclear factors of activated T- |
| | cells, IPT domain containing |
| NFATC2 | Nuclear factors of activated T- |
| | cells, IPT domain containing, |
| | MicroRNA protein coding host |
| | genes |
| NOTCH | Notch receptors, MicroRNA |
| ICD | protein coding host genes |
| REL | NF-kappa B complex subunits, |
| | IPT domain containing |
| RORA | RAR related orphan receptors |
| RUNX1 | Runt-related transcription |
| | factors |
| SATB1 | CUT class homeoboxes and |
| | pseudogenes |
| TBX21 | T-box transcription factors |
| TCF12 | Basic helix-loop-helix proteins |
| TCF3 | Basic helix-loop-helix proteins |
| TCF7 | TCF/LEF transcription factor |
| | family, Wnt enhanceosome |
| | complex |
| ZBTB7B | BTB domain containing, Zinc |
| | fingers C2H2-type |

### Example 3: TF Recipes Induced T Cell and Hematopoietic Progenitor Differentiation

In order to screen for the ability of these TF combinations to induce T cell and hematopoietic progenitor differentiation, iPSCs were transfected and antibiotics were used to select for cells that have TFs integrated into the genome. TF expression was induced by the addition of small molecules to the stem cell media for four days (96 hours). A change in morphology was observed throughout the process. No nutrient, growth factor or microenvironmental/matrix optimizations were provided to ascertain improved differentiation efficiency. These experiments demonstrated that TF expression was the only critical variable and that there were no external stimuli influencing the differentiation process.

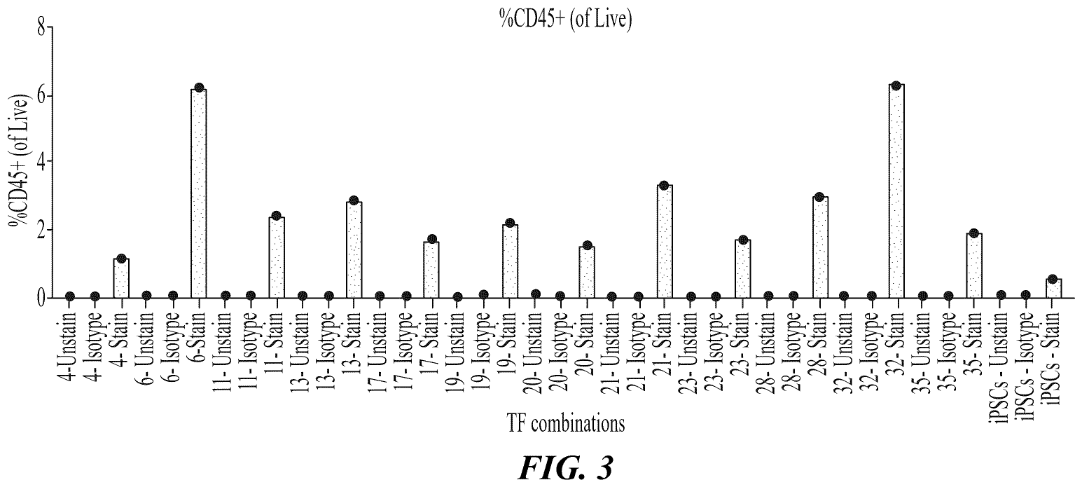

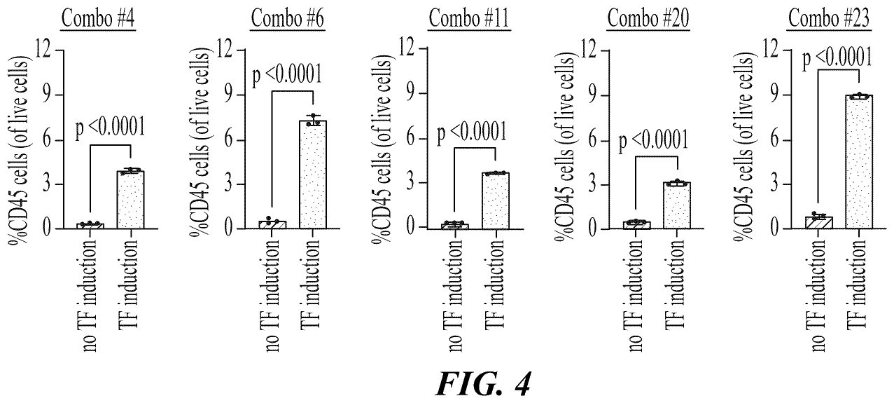

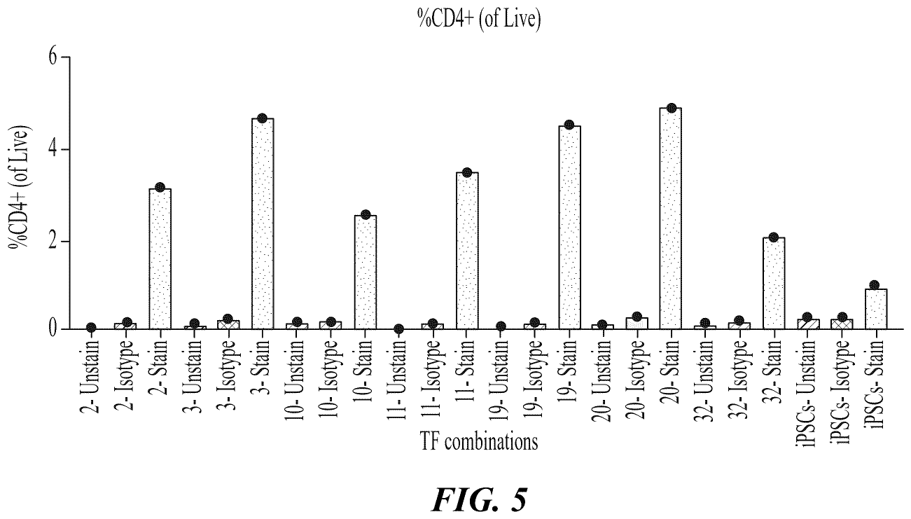

It was demonstrated that 12 combinations induced cells with CD45 expression (combinations 4, 6, 11, 13, 17, 19, 20, 21, 23, 28, 32, 35) ranging from 1% to 6% of live cells (FIG. 3), indicating cell differentiation into the hematopoietic/immune lineage. The signal was clearly distinct from cells not stained with antibodies (“Unstain”), cells stained with isotype control antibodies (“Isotype”) and cells that were not transfected with TFs (“iPSCs”). 5 TF combinations that displayed a range of percent CD45 expression (i.e. not only the top hits with highest % CD45; combinations 4, 6, 11, 20, 23) were selected to test the reproducibility and further, to validate the results. It was successfully shown that all 5 TF combinations induced expression of CD45, with the percentage of cells expressing CD45 ranging from 3% to 9% of live cells (FIG. 4). Moreover, it was discovered that 7 TF combinations that showed a range of percent expression of CD45, (2, 3, 10, 11, 19, 20, 32; FIG. 4) also induced CD4-expressing live cells, a marker indicative of T cells, ranging from 2% to 5% of live cells (FIG. 5).

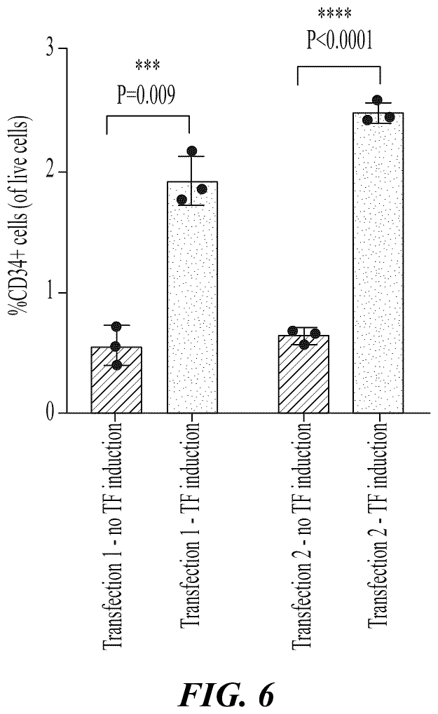

25 TFs were also tested, and cells were compared following propagation with or without TF induction for 96 hours. It was discovered that induction of various TF combination reproducibly induced cells with CD34 expression, indicating cells were able to differentiate into early hematopoietic progenitors. Overall, the expression of CD34 among live cells show that a range of 2% to 2.5% of the live cells expressed CD34 (FIG. 6). These data were reproducible in two independent transfection experiments. Overall, induction of several TF recipes or combinations led to expression of CD34 markers, some of these recipes can be seen in Table 3. Throughout the present disclosure, TF recipes, TF cocktails or TF combinations are used interchangeably.

### Example 4: TF Combinations Induced Hematopoietic Cell Lineage Differentiation

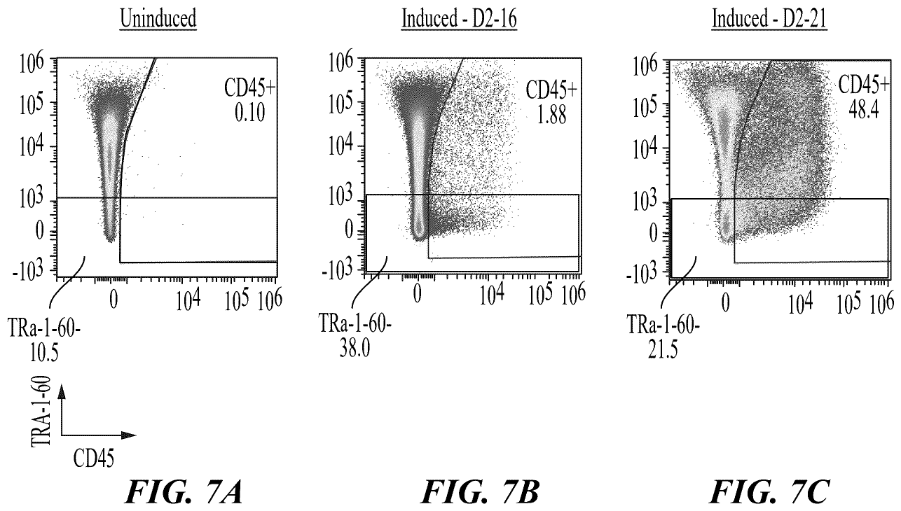

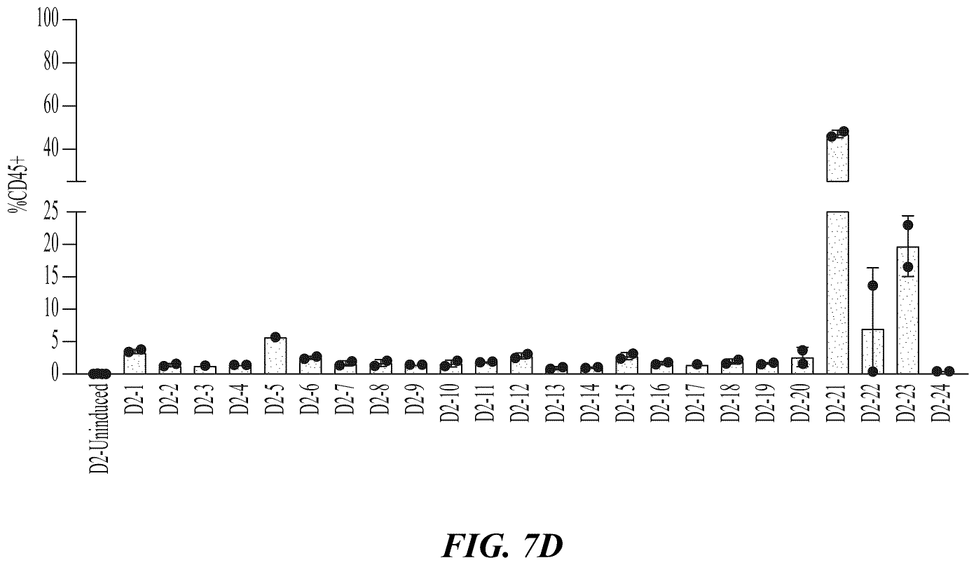

Twenty-four (24) TF combinations (Tables 5-7) were transfected in iPSCs, and TF induction and expression was allowed to proceed for 4 days (96 hours). Recipes were evaluated for ability to induce cells with CD45 expression, a marker of hematopoietic stem/progenitor cells. FIGS. 7A-7C show representative flow cytometry and corresponding gating for TRA-1-60 (iPSC marker) and CD45 (hematopoietic stem/progenitor cell lineage marker). FIG. 7D shows quantification of percentage of cells with CD45 expression in uninduced cells (D2-Uninduced) or induced cells transfected with a unique combination of TFs. The percentage of CD45 positive cells in each of the 24 TF combinations ranged from 0.5% to 46% (FIGS. 7A-FIG. 7D). Specifically, the percentage of CD45 positive (CD45+) cells detected from each TF combination tested (D2-1 to D2-24; Tables 5-Table 7) were: D2-1: 3.6%, D2-2: 1.4%, D2-3: 1.3%, D2-4: 1.5%, D2-5: 5.8%, D2-6: 2.6%, D2-7: 1.7%, D2-8: 1.7%, D2-9: 1.4%, D2-10: 2%, D2-11: 1.9%, D2-12: 2.8%, D2-13: 1.0%, D2-14: 1.0%, D2-15: 2.8%, D2-16: 1.7%, D2-17: 1.5%, D2-18: 2.0%, D2-19: 1.7%, D2-20: 2.6%, D2-21: 47.2%, D2-22: 7.1%, D2-23: 19.8%, D2-24: 0.5%. The expression of CD45 was at least 5% among iPSCs cultured with induction of TF combinations depicted in groups D2-5, D2-21, D2-22 and D2-23, with the highest expression of CD45 expressed in cells transfected with a truncated form of intracellular cellular domain of NOTCH1 (NOTCH1 ICD) of group D2-21(47%; group D2-21 Table 7), and the lowest expression in cells that received TF combinations of group D2-24 (0.5%; Table 7). Cells transfected with TF recipe in group D2-21, which contained only the NOTCH1 ICD transcription factor, indicated that 47.6% of the cells expressed CD45. These data confirmed that NOTCH1 ICD is a critical TF to the differentiation of iPSCs to cell lineages that express high levels of CD45. Since CD45 is a surface marker of immune cells, whose expression is directly proportional to the number of immune cells e.g., regulatory T cells (Treg), leukocytes, NK cells etcetera the high expression of CD45, herein, is likely associated with the high proportions (47%; group D2-21 Table 7) of iPSCs that differentiated to immune cell, e.g., Treg-expressing lineage leukocytes, NK cells etcetera.

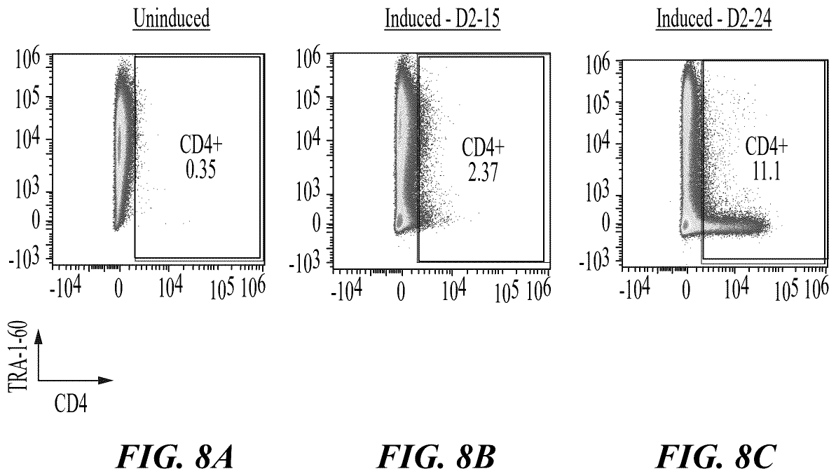

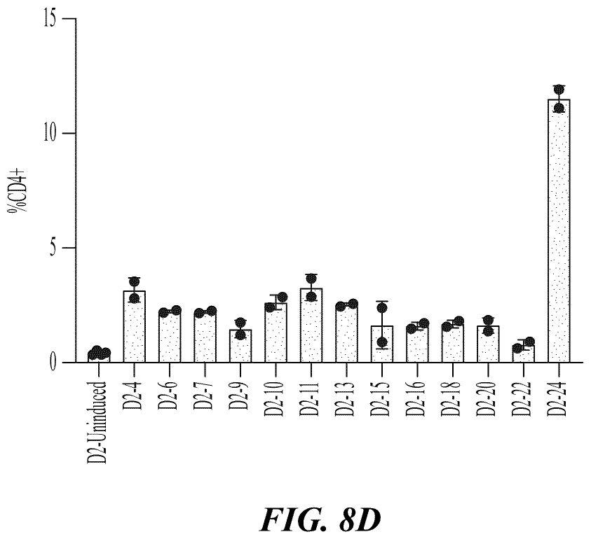

Next, a subset of 13 of the 24 TF combinations (Table 5-Table 7) were transfected in iPSCs and TF expression was induced for 96 hours, and thereafter, cells were tested for expression of CD4, a helper T-cell marker. Recipes were evaluated for ability to induce cells with CD4 expression, a marker of T helper cells. FIGS. 8A-8C show representative flow cytometry and corresponding gating for TRA-1-60 (iPSC marker) and CD4 (helper T cell marker). FIG. 8D shows quantification of percentage of cells with CD4 expression in uninduced cells (D2-Uninduced) or induced cells transfected with a unique combination of TFs. Overall, TF combination showed expression of CD4 ranging from about 1 to 12% of cells (FIGS. 8A-8D). Specifically, the percentage of CD4 positive (CD4+) cells detected were: D2-4: 3.2%, D2-6: 2.2%, D2-7: 2.2%, D2-9: 1.5%, D2-10: 2.6%, D2-11: 3.3%, D2-13: 2.5%, D2-15: 1.6%, D2-16: 1.6%, D2-18: 1.7%, D2-20: 1.6%, D2-22: 0.8%, D2-24: 11.5%. Furthermore, while the expression of CD4 was highest in iPSCs transfected with TF recipe of group D2-24 (11.5%; Table 7), it remained very low in iPSCs transfected with TF combination of group D2-22 (0.8%; Table 7). Overall, these data confirmed that TF recipe of group D2-24 is involved in the differentiation of iPSCs to CD4+ expressing (immune) cells.

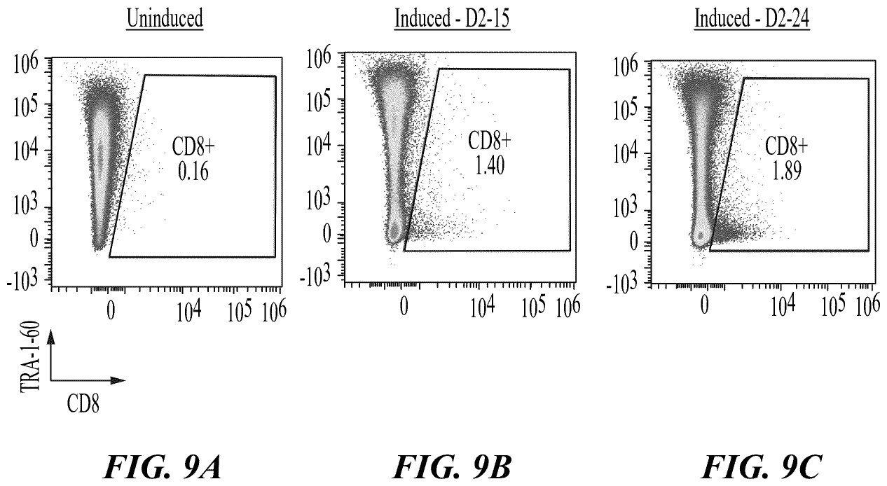

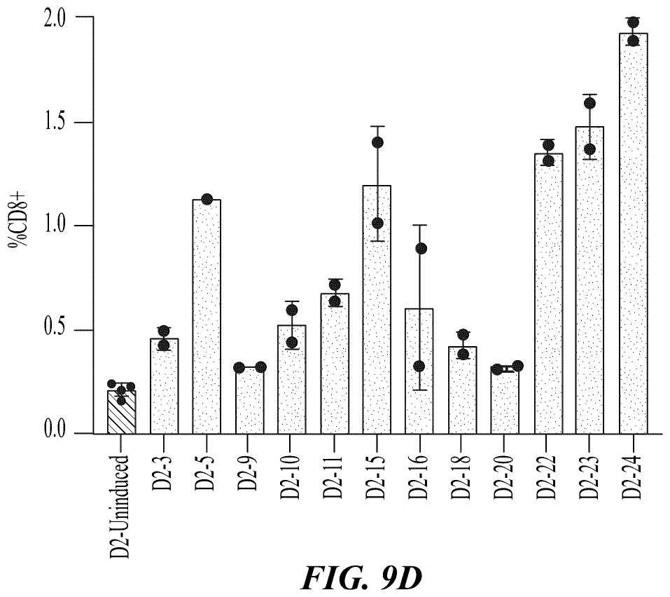

Next, a subset of 12 of 24 TF combinations (in Table 5-Table 7) was transfected in iPSCs and TF expression was induced for 96 hours. Recipes were test for ability to induce cells with CD8 expression, a marker of cytotoxic T cells. FIGS. 9A-9C shows representative flow cytometry and corresponding gating for marker TRA-1-60 (iPSC marker) and CD8 (cytotoxic T cell marker). FIG. 9D shows quantification of percentage of cells with CD8 expression in uninduced cells (D2-Uninduced) or induced cells transfected with a unique combination of TFs. Overall, TF combinations showed expression of CD8 ranging from about 0.3 to 200 of cells (FIGS. 9A-9D). Specifically, the percentage of CD8 positive (CD8+) cells detected were: D2-3: 0.46%, D2-5: 1.13%, D2-9: 0.32%, D2-10: 0.52%, D2-11: 0.68%, D2-15: 1.21%, D2-16: 0.61, D2-18: 0.43, D2-20: 0.32, D2-22: 1.35, D2-23: 1.48, D2-24: 1.94%. These data suggest that the specific TF combinations transfected in iPSCs induced cells to differentiate to CD8+ immune cells.

TABLE 5
List of transcription factor combinations found in D2 groups (TF combinations
associated with hematopoietic lineage differentiation)

| D2-1 | D2-2 | D2-3 | D2-4 | D2-5 | D2-6 | D2-7 | D2-8 |
|---|---|---|---|---|---|---|---|
| FOXP3 | IKZF1 | BCL11B | BCL11B | BCL11B | BCL11B | DTX1 | BCL11B |
| GATA3 | IKZF2 | DTX1 | DTX1 | IKZF1 | IKZF1 | IKZF2 | DTX1 |
| NFATC1 | FOXO1 | IKZF1 | IKZF1 | GATA3 | IKZF2 | FOXO1 | IKZF1 |
| NOTCH1 | FOXO3 | IKZF2 | IKZF2 | KLF10 | FOXO1 | FOXO3 | IKZF2 |
| ICD | | | | | | | |
| TBX21 | FOXP3 | IRF4 | FOXO1 | NFATC1 | FOXP3 | GATA3 | FOXO1 |
| TCF12 | GATA3 | FOXO1 | FOXO3 | NFATC2 | GATA1 | LEF1 | FOXO3 |
| TCF7 | KLF10 | FOXP3 | FOXP3 | NOTCH1 | GATA3 | NFATC2 | FOXP3 |
| | | | | ICD | | | |
| ZBTB7B | LEF1 | GATA1 | GATA3 | RORA | KLF10 | NOTCH1 | GATA3 |
| | | | | | | ICD | |
| | NFATC2 | KLF10 | KLF10 | SATB1 | NFATC2 | REL | NFATC2 |
| | NOTCH1 | LEF1 | LEF1 | TBX21 | NOTCH1 | SATB1 | NOTCH1 |
| | ICD | | | | ICD | | ICD |
| | SATB1 | NOTCH1 | NFATC1 | TCF7 | SATB1 | TBX21 | RORA |
| | | ICD | | | | | |
| | TBX21 | SATB1 | NFATC2 | | TBX21 | TCF12 | REL |
| | TCF7 | TCF12 | NOTCH1 | | TCF12 | | SATB1 |
| | | | ICD | | | | |
| | ZBTB7B | | REL | | TCF7 | | TBX21 |
| | | | | | ZBTB7B | | TCF12 |
| | | | | | | | TCF7 |
| | | | | | | | ZBTB7B |

TABLE 6
List of transcription factor combinations found in D2 groups (TF combinations
associated with hematopoietic lineage differentiation)

| D2-9 | D2-10 | D2-11 | D2-12 | D2-13 | D2-14 | D2-15 | D2-16 |
|---|---|---|---|---|---|---|---|
| BCL11B | BCL11B | BCL11B | BCL11B | BCL11B | BCL11B | BCL11B | BCL11B |
| FOXP3 | DTX1 | DTX1 | DTX1 | IKZF1 | IKZF2 | IKZF1 | DTX1 |
| GATA1 | IKZF1 | IKZF1 | FOXO1 | IKZF2 | FOXO1 | IKZF2 | IKZF1 |
| GATA3 | IKZF2 | IKZF2 | FOXO3 | FOXO1 | FOXP3 | FOXP3 | IKZF2 |
| KLF10 | FOXO1 | IRF4 | FOXP3 | FOXO3 | GATA3 | GATA1 | FOXO1 |
| LEF1 | FOXO3 | FOXP3 | GATA1 | GATA3 | KLF10 | KLF10 | FOXO3 |
| NOTCH1 | FOXP3 | GATA1 | GATA3 | KLF10 | LEF1 | NFATC1 | FOXP3 |
| ICD | | | | | | | |
| RORA | GATA3 | GATA3 | NOTCH1 | LEF1 | NFATC2 | NOTCH1 | GATA3 |
| | | | ICD | | | ICD | |
| SATB1 | KLF10 | KLF10 | SATB1 | NFATC2 | NOTCH1 | RORA | KLF1 |
| | | | | | ICD | | |
| TBX21 | LEF1 | LEF1 | TCF12 | NOTCH1 | SATB1 | REL | LEF1 |
| | | | | ICD | | | |
| TCF3 | NFATC2 | NOTCH1 | | REL | TCF12 | RUNX1 | NOTCH1 |
| | | ICD | | | | | ICD |
| TCF12 | NOTCH1 | TCF7 | | TCF12 | TCF7 | TBX21 | RORA |
| | ICD | | | | | | |
| ZBTB7B | RORA | | | TCF7 | | TCF3 | REL |
| | SATB1 | | | ZBTB7B | | TCF7 | SATB1 |
| | | | | | | ZBTB7B | TCF7 |
| | | | | | | | ZBTB7B |

TABLE 7
List of transcription factor combinations found in D2 groups (TF combinations
associated with hematopoietic lineage differentiation)

| D2-17 | D2-18 | D2-19 | D2-20 | D2-21 | D2-22 | D2-23 | D2-24 |
|---|---|---|---|---|---|---|---|
| DTX1 | BCL11B | BCL11B | BCL11B | NOTCH1 | IRF4 | NOTCH1 | NOTCH1 |
| | | | | ICD | | ICD | ICD |
| FOXO1 | DTX1 | DTX1 | IKZF2 | | NOTCH1 | TCF3 | RUNX1 |
| | | | | | ICD | | |
| FOXO3 | IKZF1 | FOXO1 | FOXO1 | | | | TCF3 |
| FOXP3 | FOXO3 | GATA1 | FOXO3 | | | | |
| GATA1 | GATA1 | KLF1 | FOXP3 | | | | |
| GATA3 | GATA3 | NFATC1 | GATA3 | | | | |
| NFATC1 | LEF1 | NFATC2 | NFATC2 | | | | |
| NOTCH1 | NOTCH1 | NOTCH1 | NOTCH1 | | | | |
| ICD | ICD | ICD | ICD | | | | |
| REL | RORA | REL | RORA | | | | |
| SATB1 | SATB1 | SATB1 | REL | | | | |
| TBX21 | TBX21 | TBX21 | TBX21 | | | | |
| TCF12 | TCF12 | TCF12 | TCF7 | | | | |
| TCF7 | ZBTB7B | TCF7 | ZBTB7B | | | | |

### Example 5: Using NOTCH1 ICD in Addition with Other TF to Induce Differentiation of iPSCs

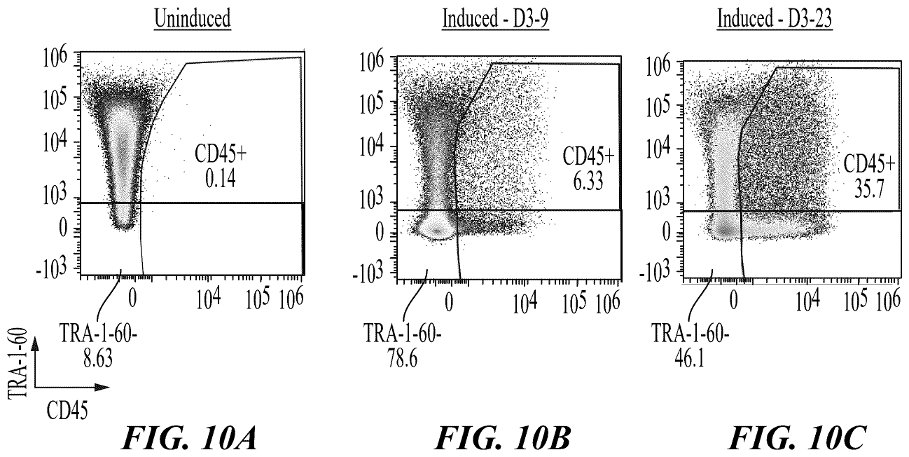

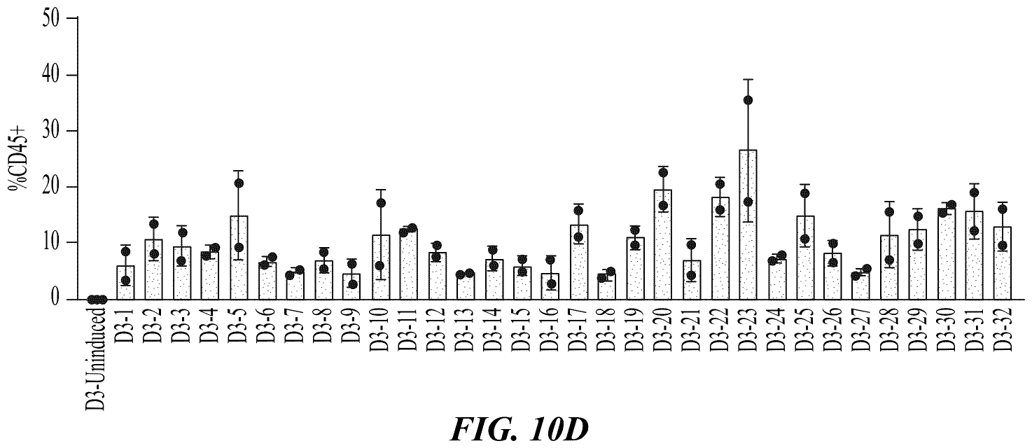

This experiment further investigated NOTCH1 ICD in combination with other transcription factors (TF) in inducing differentiation of hematopoietic cell lineage, to immune cells by e.g., an expression of CD45, CD8 immune cell markers. Thirty-two (32) additional TF combinations (designated D3-1 to D3-32; Tables 8-11) were transfected in iPSCs and TF induction and expression was allowed to proceed for 4 days (96 hours). Recipes were evaluated for ability to induce cells with CD45 expression, a marker of hematopoietic stem/progenitor cells. FIGS. 10A-10C show representative flow cytometry and corresponding gating for marker TRA-1-60 (iPSC marker) and CD45 (hematopoietic lineage marker). FIG. 10D shows quantification of percentage of cells with CD45 expression in uninduced cells (D3-Uninduced) or induced cells transfected with combinations of TFs. The percentage of CD45 positive cells in each of the 32 TF combinations ranged from 4% to 27% (FIG. 10D). Specifically, the percentage of CD45 positive (CD45+) cells detected from each TF combination tested (D3-1 to D3-32; Tables 8-Table 11) were: D3-1: 6.0%, D3-2: 10.8%, D3-3: 9.5%, D3-4: 8.4%, D3-5: 14.9%, D3-6: 6.7%, D3-7: 4.8%, D3-8: 6.9%, D3-9: 4.6%, D3-10: 11.6%, D3-11: 12.4%, D3-12: 8.4%, D3-13: 4.4%, D3-14: 7.2%, D3-15: 5.8%, D3-16: 4.7%, D3-17: 13.8%, D3-18: 4.3%, D3-19: 11.0%, D3-20: 19.7%, D3-21: 7.0%, D3-22: 18.3%, D3-23: 26.7%, D3-24: 7.2%, D3-25: 14.8%, D3-26: 8.2%, D3-27: 4.8%, D3-28: 11.5%, D3-29: 12.4%, D3-30: 16.3%, D3-31: 15.6%, D3-32: 13%. With the additional batch of 32 TF combinations, nearly 94% (27/32) TF combinations induced about 5% of cells to differentiate into immune cells that expressed CD45. In fact, 50% (16/32) of TF combinations were able to induce differentiation of cell such that 10% of cells were CD45 positive, while 22% (7/32) TF combinations induced iPSCs to differentiate so that >15% of cells expressed CD45 immune cell marker. Among the TF combination, those of group D3-23 (Table 10; FIG. 10C-10D) showed highest induction with about 27% cells displaying CD45 expression. These data indicate, TF recipe of group D3-23 is critical to the induction of hematopoietic cell lineage to differentiate to CD45-expressing immune cells e.g., Tregs, leukocytes, natural killer cells (NK cell).

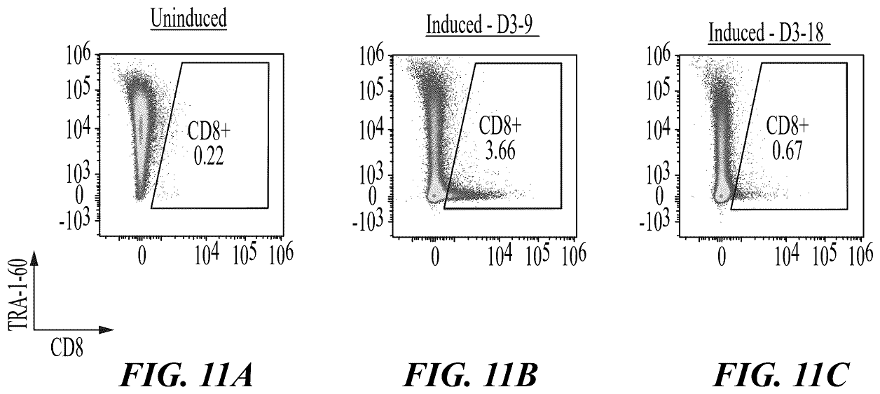

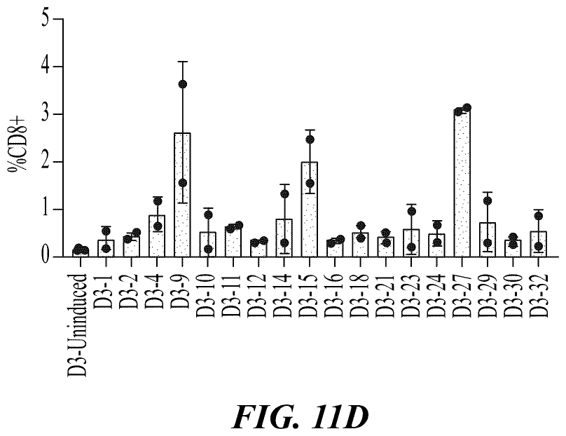

Next, a subset of 18 of the 32 TF combinations (subset of TF recipes of groups D3-1 to D3-32 in Table 8-Table 11) was transfected in iPSCs and TF expression was induced for 96 hours. Recipes were evaluated for ability to induce cells with CD8 expression, a marker of cytotoxic T cells. FIGS. 11A-11C show representative flow cytometry and corresponding gating for marker TRA-1-60 (iPSC marker) and CD8 (cytotoxic T cell marker). FIG. 11D shows quantification of percentage of cells with CD8 expression in TF uninduced cells (D3-Uninduced) or induced cells transfected with a unique combination of TFs. The TF combination showed expression of CD8, a cytotoxic T cell marker ranging from about 0.5 to 4% of cells (FIGS. 11A-11D). Specifically, the percent of cells expressing CD8 were: D3-1: 0.37%, D3-2: 0.44%, D3-4: 0.91%, D3-9: 2.62%, D3-10: 0.52%, D3-11: 0.63%, D3-12: 0.33%, D3-14: 0.82%, D3-15: 2.03%, D3-16: 0.34%, D3-18: 0.54%, D3-21: 0.42%, D3-23: 0.6%, D3-24: 0.5%, D3-27: 3.1%, D3-29: 0.75%, D3-30: 0.34%, D3-32: 0.55%. Cells transfected with TF combinations of groups D3-9, D3-15 and D3-27 displayed the highest expression of about 2.6%, 2% and 3% expression of CD8+ cells, respectively. These data suggest that the specific TF combinations D3-9, D3-15 and D3-27 transfected in iPSCs induced 2-3% of cells to differentiate to immune cells that express CD8+ cell marker.

### Example 6: Confirmation of TF Recipe that Induces Differentiation of iPSCs to Immune Lineages

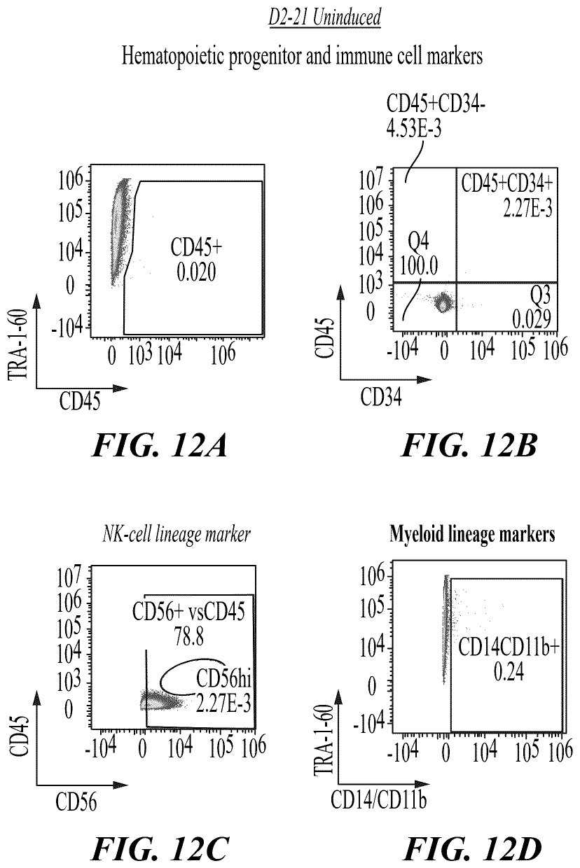

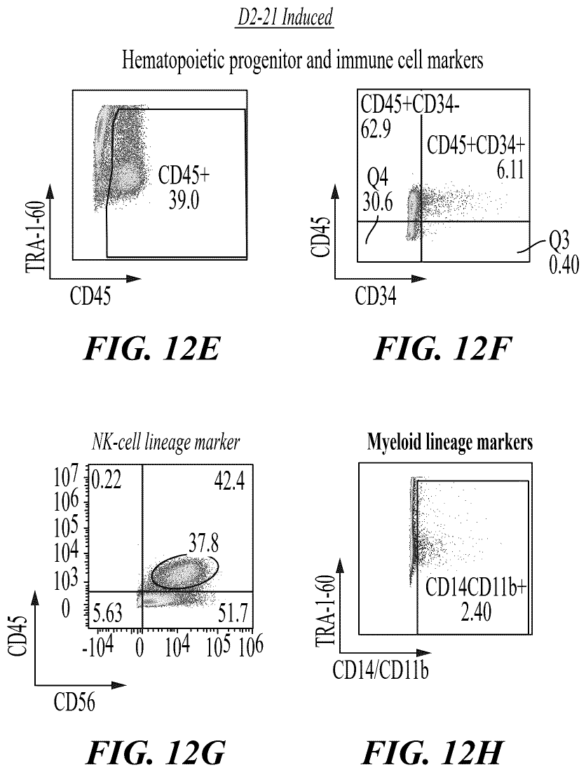

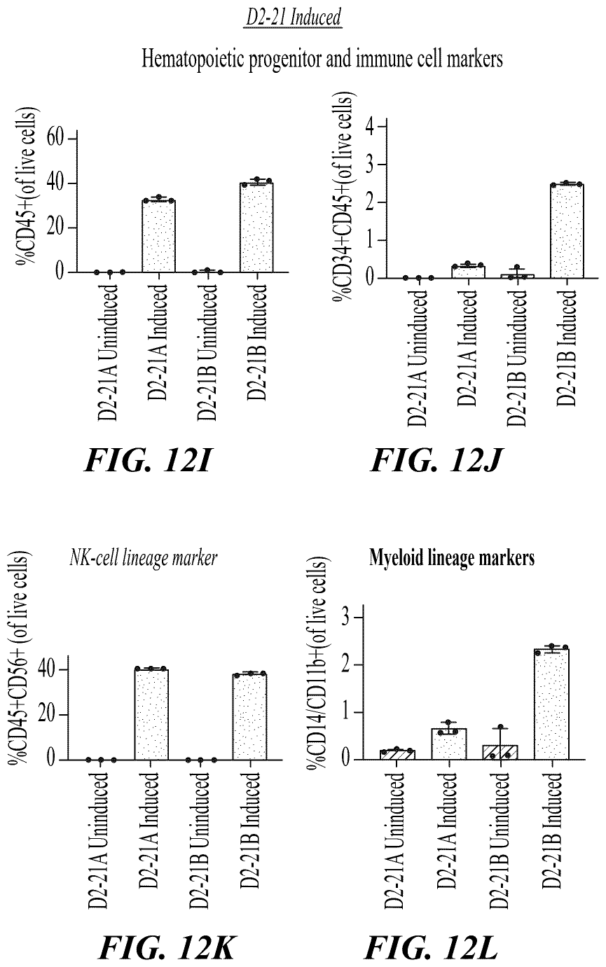

In this experiment, an investigation was conducted on the immune lineages formed in TF recipe with the highest percentage of cells expressing CD45 (D2-21). Recipe D2-21 generates distinct immune subtypes. D2-21A and D2-21B are the recipe in two different human iPSC lines. Further, TF recipe D2-21 contains 1 transcription factor, a truncated form of intracellular cellular domain of NOTCH1 (NOTCH1 ICD), that earlier produced the highest percentage (˜47%; Table 7) of cells expressing CD45. The amino acid sequence for the NOTCH1 ICD, SEQ ID NO.: 1, is shown in Table 12. As noted above, because NOTCH1 ICD produced the highest proportion of cells that expressed CD45, a marker of immune cells, confirmed that NOTCH1 ICD is a critical TF for differentiation of iPSCs to immune cells. Specifically, for this particular experiment, TFs were evaluated in the NOTCH1 ICD cell line for proportion and ability of TF recipes to induce cells expressing different immune subtype markers (singly or double positives) after 96 hours of TF induction and expression was investigated. FIGS. 12A-12H show representative flow cytometry and corresponding gating for uninduced (FIGS. 12A-12D) and induced cells (FIGS. 12E-12H): TRA-1-60 (iPSC marker), CD45 (hematopoietic lineage marker), CD34 (hematopoietic stem/progenitor cell marker), CD56 (NK cell marker), CD14/CD11b (myeloid marker). FIGS. 12I-12L shows quantification of percentage of cells (a) CD45 expression (41% of live cells; FIG. 12I), (b) CD34+CD45+ double positive expression (2.5% of live cells, FIG. 12J), (c) CD45+CD56+ double positive expression (40% of live cells; FIG. 12K), (d) CD14+/CD11b+ double positive expression (2.3% of live cells; FIG. 12L).

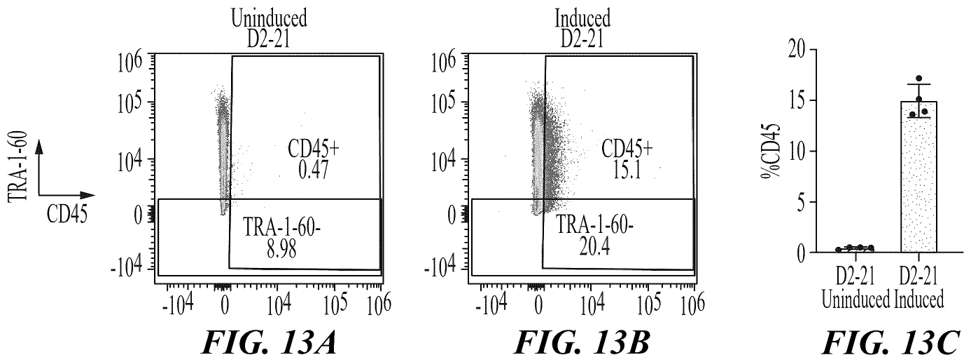

Next, recipe D2-21 was confirmed to induce expression of CD45 as early as 24 hours. FIGS. 13A-13B show representative flow plots and gating strategy. FIG. 13C shows quantification of CD45 expression in uninduced and induced cells. Notably, in as early as 24 hours of TF expression, about 15% of cells expressed CD45 (pan-hematopoietic marker, FIGS. 13B-13C), and as expected, there was no expression when TF expression was uninduced (FIGS. 13A and 13C).

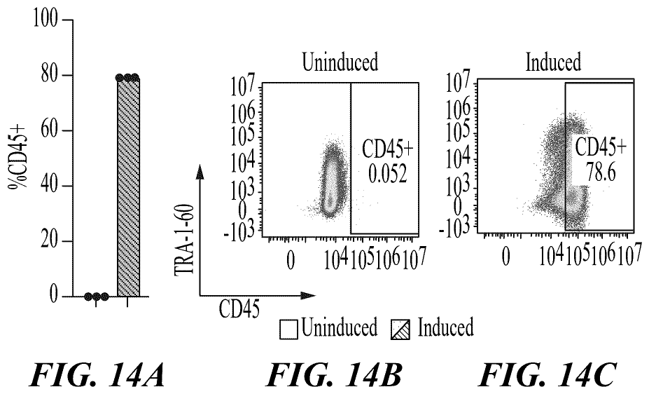

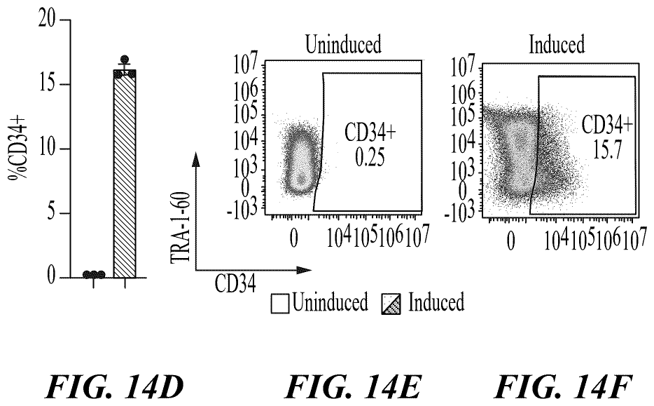

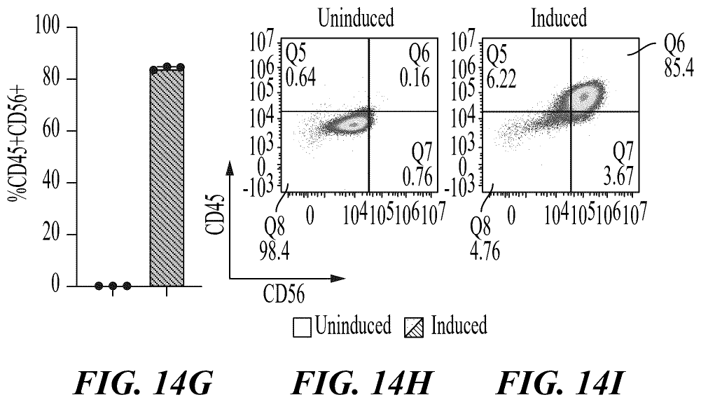

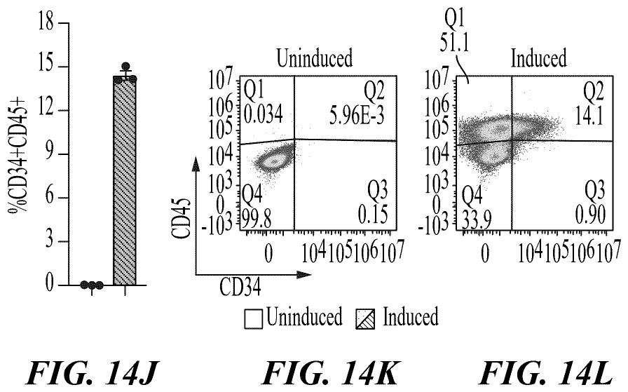

Next, different top performing cell lines were isolated, differentiated for 96 hours and assessed. Top performers isolated from the recipe D2-21 cell line had higher CD45+, CD45+CD56+ double positive, CD34+ or CD34+CD45+ double positive differentiation efficiency. FIGS. 14A, 14D, 14G and 14J show representative flow cytometry for percentage of cells expressing the indicated marker(s). Corresponding gating the indicated markers are shown in FIGS. 14B-14C; 14E-14F and 14H-14I and 14K-14L. The hematopoietic marker CD45 was expressed in up to ˜80% of induced cells. CD45+CD56+ double positive marker of natural killer (NK) cells was expressed in up to ˜80% of induced cells. The hematopoietic stem/progenitor marker CD34 was expressed in up to ˜16% of induced cells. The CD34+CD45+ double positive stringent marker of hematopoietic stem/progenitor cells was expressed in up to ˜14% of induced cells.

TABLE 8
List of transcription factor combinations

| D3-1 | D3-2 | D3-3 | D3-4 | D3-5 | D3-6 | D3-7 | D3-8 |
|---|---|---|---|---|---|---|---|
| NOTCH1 | BCL11B | DTX1 | BCL11B | IKZF4 | BCL11B | DTX1 | BCL11B |
| ICD | | | | | | | |
| REL | FOXP3 | FOXP3 | DTX1 | FOXP3 | IKZF4 | IKZF4 | DTX1 |
| RUNX1 | IKZF1 | GATA3 | GATA3 | GATA3 | GATA3 | GATA1 | IKZF4 |
| SATB1 | IKZF2 | IRF4 | IKZF1 | GATA1 | GATA1 | IKZF1 | FOXP3 |
| TBX21 | KLF10 | KLF10 | IKZF2 | IKZF1 | IKZF2 | IRF4 | GATA1 |
| TCF12 | LEF1 | LEF1 | IRF4 | NFATC1 | KLF10 | KLF10 | IKZF2 |
| TCF3 | NFATC1 | NOTCH1 | NFATC1 | NOTCH1 | LEF1 | LEF1 | IRF4 |
| | | ICD | | ICD | | | |
| TCF7 | NOTCH1 | MEF2C | NOTCH1 | MEF2C | NOTCH1 | NFATC1 | NOTCH1 |
| | ICD | | ICD | | ICD | | ICD |
| ZBTB7B | RORA | RORA | MEF2C | RORA | MEF2C | NOTCH1 | RORA |
| | | | | | | ICD | |
| | TCF12 | TBX21 | REL | SATB1 | REL | REL | SATB1 |
| | | TCF7 | RUNX1 | TCF3 | RUNX1 | RUNX1 | TBX21 |
| | | ZBTB7B | SATB1 | ZBTB7B | TBX21 | TCF12 | TCF12 |
| | | | TCF3 | | TCF7 | ZBTB7B | TCF3 |

TABLE 9
List of transcription factor combinations

| D3-9 | D3-10 | D3-11 | D3-12 | D3-13 | D3-14 | D3-15 | D3-16 |
|---|---|---|---|---|---|---|---|
| JUN | BCL11B | DTX1 | BCL11B | IKZF4 | BCL11B | DTX1 | BCL11B |
| GATA3 | JUN | JUN | DTX1 | JUN | IKZF4 | IKZF4 | DTX1 |
| GATA1 | FOXP3 | FOXP3 | JUN | FOXP3 | JUN | JUN | IKZF4 |
| IKZF1 | GATA3 | GATA1 | GATA1 | IKZF2 | IKZF1 | GATA3 | JUN |
| IKZF2 | GATA1 | IKZF1 | KLF10 | IRF4 | IRF4 | IKZF2 | FOXP3 |
| IRF4 | IRF4 | IKZF2 | NFATC1 | KLF10 | LEF1 | LEF1 | GATA3 |
| KLF10 | LEF1 | LEF1 | NOTCH1 | NFATC1 | NOTCH1 | NFATC1 | IKZF1 |
| | | | ICD | | ICD | | |
| NOTCH1 | NFATC1 | NOTCH1 | MEF2C | NOTCH1 | MEF2C | NOTCH1 | KLF10 |
| ICD | | ICD | | ICD | | ICD | |
| RORA | NOTCH1 | MEF2C | RORA | MEF2C | RORA | RORA | NOTCH1 |
| | ICD | | | | | | ICD |
| RUNX1 | REL | REL | RUNX1 | REL | RUNX1 | RUNX1 | REL |
| TCF3 | SATB1 | SATB1 | TBX21 | TBX21 | SATB1 | SATB1 | TCF3 |
| TCF7 | TBX21 | TCF12 | TCF12 | TCF12 | TCF12 | TBX21 | TCF7 |
| | ZBTB7B | TCF7 | TCF3 | TCF3 | TCF7 | | ZBTB7B |

TABLE 10
List of transcription factor combinations found in D3 groups

| D3-17 | D3-18 | D3-19 | D3-20 | D3-21 | D3-22 | D3-23 | D3-24 |
|---|---|---|---|---|---|---|---|
| FOXO3 | BCL11B | DTX1 | BCL11B | IKZF4 | BCL11B | DTX1 | BCL11B |
| GATA1 | FOXO3 | FOXO3 | DTX1 | FOXO3 | IKZF4 | IKZF4 | DTX1 |
| IKZF2 | FOXP3 | FOXP3 | FOXO3 | FOXP3 | FOXO3 | FOXO3 | IKZF4 |
| IRF4 | GATA1 | GATA3 | GATA3 | GATA3 | GATA3 | IKZF1 | FOXO3 |
| LEF1 | IKZF1 | GATA1 | GATA1 | IKZF1 | IRF4 | IKZF2 | FOXP3 |
| NFATC1 | IRF4 | IKZF2 | IKZF1 | IKZF2 | KLF10 | KLF10 | LEF1 |
| NOTCH1 | KLF10 | KLF10 | LEF1 | IRF4 | NFATC1 | NOTCH1 | NFATC1 |
| ICD | | | | | | ICD | |
| MEF2C | NOTCH1 | NFATC1 | NOTCH1 | LEF1 | NOTCH1 | MEF2C | NOTCH1 |
| | ICD | | ICD | | ICD | | ICD |
| RORA | MEF2C | NOTCH1 | RORA | NOTCH1 | RORA | RORA | MEF2C |
| | | ICD | | ICD | | | |
| REL | RUNX1 | RUNX1 | REL | RUNX1 | REL | REL | RUNX1 |
| TCF3 | SATB1 | SATB1 | TBX21 | TBX21 | SATB1 | SATB1 | TCF3 |
| TCF7 | TBX21 | TCF12 | TCF12 | TCF12 | TCF12 | TBX21 | TCF7 |
| ZBTB7B | | TCF7 | TCF3 | TCF3 | TCF7 | ZBTB7B | |
| | | ZBTB7B | | ZBTB7B | | | |

TABLE 11
List of transcription factor combinations found in D3 groups

| D3-25 | D3-26 | D3-27 | D3-28 | D3-29 | D3-30 | D3-31 | D3-32 |
|---|---|---|---|---|---|---|---|
| JUN | BCL11B | DTX1 | BCL11B | IKZF4 | BCL11B | DTX1 | BCL11B |
| FOXO3 | JUN | JUN | DTX1 | JUN | IKZF4 | IKZF4 | DTX1 |
| GATA3 | FOXO3 | FOXO3 | JUN | FOXO3 | JUN | JUN | IKZF4 |
| IKZF1 | FOXP3 | FOXP3 | FOXO3 | FOXP3 | FOXO3 | FOXO3 | JUN |
| KLF10 | GATA3 | IKZF1 | IKZF2 | GATA1 | GATA1 | GATA3 | FOXO3 |
| LEF1 | IKZF2 | IRF4 | IRF4 | KLF10 | IKZF1 | GATA1 | FOXP3 |
| NFATC1 | NOTCH1 | NFATC1 | KLF10 | LEF1 | IKZF2 | IRF4 | GATA3 |
| | ICD | | | | | | |
| NOTCH1 | MEF2C | NOTCH1 | LEF1 | NOTCH1 | NFATC1 | NOTCH1 | GATA1 |
| ICD | | ICD | | ICD | | ICD | |
| MEF2C | RORA | RORA | NOTCH1 | RORA | NOTCH1 | MEF2C | IKZF1 |
| | | | ICD | | ICD | | |
| SATB1 | REL | REL | SATB1 | REL | TBX21 | TCF12 | IKZF2 |
| TBX21 | RUNX1 | RUNX1 | TCF3 | RUNX1 | TCF7 | | IRF4 |
| TCF12 | TCF12 | TBX21 | ZBTB7B | SATB1 | ZBTB7B | | KLF10 |
| TCF3 | ZBTB7B | TCF7 | | TCF3 | | | LEF1 |
| TCF7 | | | | | | | NFATC1 |
| | | | | | | | NOTCH1 |
| | | | | | | | ICD |
| | | | | | | | MEF2C |
| | | | | | | | RORA |
| | | | | | | | REL |
| | | | | | | | RUNX1 |
| | | | | | | | SATB1 |
| | | | | | | | TBX21 |
| | | | | | | | TCF12 |
| | | | | | | | TCF3 |
| | | | | | | | TCF7 |
| | | | | | | | ZBTB7B |

TABLE 12
NOTCH1 ICD protein sequence

| SEQ ID | |
|---|---|
| NO. | Sequence |
| 1 | MVLLSRKRRRQHGQLWFPEGFKVSEASKKK |
| | RREPLGEDSVGLKPLKNASDGALMDDNQNE |
| | WGDEDLETKKFRFEEPVVLPDLDDQTDHRQ |
| | WTQQHLDAADLRMSAMAPTPPQGEVDADCM |
| | DVNVRGPDGFTPLMIASCSGGGLETGNSEE |
| | EEDAPAVISDFIYQGASLHNQTDRTGETAL |
| | HLAARYSRSDAAKRLLEASADANIQDNMGR |
| | TPLHAAVSADAQGVFQILIRNRATDLDARM |
| | HDGTTPLILAARLAVEGMLEDLINSHADVN |
| | AVDDLGKSALHWAAAVNNVDAAVVLLKNGA |
| | NKDMQNNREETPLFLAAREGSYETAKVLLD |
| | HFANRDITDHMDRLPRDIAQERMHHDIVRL |
| | LDEYNLVRSPQLHGAPLGGTPTLSPPLCSP |
| | NGYLGSLKPGVQGKKVRKPSSKGLACGSKE |
| | AKDLKARRKKSQDGKGCLLDSSGMLSPVDS |
| | LESPHGYLSDVASPPLLPSPFQQSPSVPLN |
| | HLPGMPDTHLGIGHLNVAAKPEMAALGGGG |
| | RLAFETGPPRLSHLPVASGTSTVLGSSSGG |
| | ALNFTVGGSTSLNGQCEWLSRLQSGMVPNQ |
| | YNPLRGSVAPGPLSTQAPSLQHGMVGPLHS |
| | SLAASALSQMMSYQGLPSTRLATQPHLVQT |
| | QQVQPQNLQMQQQNLQPANIQQQQSLQPPP |
| | PPPQPHLGVSSAASGHLGRSFLSGEPSQAD |
| | VQPLGPSSLAVHTILPQESPALPTSLPSSL |
| | VPPVTAAQFLTPPSQHSYSSPVDNTPSHQL |
| | QVPEHPFLTPSPESPDQWSSSSPHSNVSDW |
| | SEGVSSPPTSMQSQIARIPEAFK |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1-138. (canceled)

139. A population of engineered cells configured to express at least two immune cell markers in a time period of 96 hours or less after induction of a plurality of transcription factors.

140. The population of engineered cells of claim 139, wherein said at least two immune cell markers comprise at least two of FOXP3, CD34, CD45, CD4, CD8, CD14, and CD11b.

141. The population of engineered cells of claim 139, wherein at least 5% of said population of engineered cells express FOXP3.

142. The population of engineered cells of claim 139, wherein at least 1% of said population of engineered cells express CD45.

143. The population of engineered cells of claim 139, wherein at least 1% of said population of engineered cells express CD8.

144. The population of engineered cells of claim 139, wherein at least 1% of said population of engineered cells express CD34.

145. The population of engineered cells of claim 139, wherein at least 2% of said population of engineered cells express CD4.

146. The population of engineered cells of claim 139, wherein at least 1% of said population of engineered cells express CD45 and CD34.

147. The population of engineered cells of claim 139, wherein at least 5% of said population of engineered cells express CD45 and CD56.

148. The population of engineered cells of claim 139, wherein at least 5% of said population of engineered cells express CD45 and CD34.

149. The population of engineered cells of claim 139, wherein at least 1% of said population of engineered cells express CD14 and CD11b.

150. A method of generating a population of engineered cells, said method comprising providing a plurality of pluripotent stem cells (PSCs), wherein a PSC of said plurality of PSCs comprises two or more transcription factors, wherein said two or more transcription factors cause said PSC to differentiate into an engineered cell, wherein said engineered cell expresses at least two immune cell markers in a time period of 96 hours or less.

151. The method of claim 150, wherein said at least two immune cell markers comprise at least two of FOXP3, CD34, CD45, CD4, CD8, CD14, and CD11b.

152. The method of claim 150, wherein at least 5% of said population of engineered cells express FOXP3.

153. The method of claim 150, wherein at least 1% of said population of engineered cells express CD8.

154. The method of claim 150, wherein at least 1% of said population of engineered cells express CD45.

155. The method of claim 150, wherein at least 1% of said population of engineered cells express CD34.

156. The method of claim 150, wherein at least 2% of said population of engineered cells express CD4.

157. The method of claim 150, wherein at least 1% of said population of engineered cells express CD45 and CD34.

158. The method of claim 150, wherein at least 5% of said population of engineered cells express CD45 and CD56.

159. The method of claim 150, wherein at least 5% of said population of engineered cells express CD45 and CD34.

160. The method of claim 150, wherein at least 1% of said population of engineered cells express CD14 and CD11b.


SEQUENCE LISTING (1 sequence)

SEQ ID NO: 1 - protein, 803 aa, synthetic construct
MVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK